(19) United States
(12) Patent Application Publication
(10) Pub. No.: US 2017/0095457 A1
(43) Pub. Date: Apr. 6, 2017

(54) COMPOSITIONS AND METHODS OF DELIVERY OF DEUBIQUITINASE INHIBITORS

(71) Applicants: David LONERGAN, San Marcos, CA (US); PharmAkea, Inc., San Diego, CA (US)

(72) Inventors: David LONERGAN, San Marcos, CA (US); John Howard HUTCHINSON, San Diego, CA (US)

(21) Appl. No.: 15/311,178

(22) PCT Filed: May 27, 2015
(86) PCT No.: PCT/US15/32718

§ 371 Date: Nov. 14, 2016

Related U.S. Application Data
(60) Provisional application No. 62/003,363, filed on May 27, 2014.

(51) Int. Cl. A61K 31/44; A61K 9/00

(52) U.S. Cl. CPC A61K 31/44; A61K 47/48284; A61K 47/48853; A61K 47/48338; A61K 9/0019

(57) ABSTRACT

Described herein are compositions comprising deubiquitinase inhibitors in combination with albumin, methods of making such compositions, and methods of using such compositions in the treatment of conditions, diseases, or disorders that would benefit from inhibition of deubiquitinase activity.

### RELATED APPLICATIONS

This application claims the benefit of U.S. Provisional Patent Application No. 62/003,363 entitled “COMPOSITIONS AND METHODS OF DELIVERY OF DEUBIQUITINASE INHIBITORS” filed on May 27, 2014, which is incorporated herein by reference in its entirety.

### FIELD OF THE INVENTION

Described herein are pharmaceutical compositions comprising deubiquitinase inhibitors and human serum albumin, methods of making such pharmaceutical compositions, and methods of using such pharmaceutical compositions in the treatment of conditions, diseases, or disorders that would benefit from inhibition of deubiquitinase activity.

### BACKGROUND OF THE INVENTION

Ubiquitination is a covalent posttranslational modification of cellular proteins involving a complex enzymatic cascade. Enzymes of the ubiquitination cascade are differentially expressed or activated in many diseases, including cancer. Protein ubiquitination is a dynamic two-way process that can be reversed or regulated by deubiquitinating enzymes (DUB). DUBs primarily serve to counterbalance ubiquitin-protein conjugation and also facilitate the cleavage of ubiquitin from its precursors and unanchored polyubiquitin chains. Thus, DUBs regulate and maintain the homeostasis of free ubiquitin pools in the cell. DUBs enhance protein stability by preventing protein degradation and dysregulation in the activity and expression of DUBs has been linked to cancer development and progression.

### SUMMARY OF THE INVENTION

In one aspect, described herein is a method of treating a disease or condition in a mammal that would benefit from the inhibition of the activity of a deubiquitinating enzyme comprising administering to the mammal a pharmaceutical composition comprising a deubiquitinase inhibitor and albumin. In some embodiments, the disease or condition is cancer, fibrosis, an autoimmune disease or condition, an inflammatory disease or condition, a neurodegenerative disease or condition or an infection.

In another aspect, described herein is a method of enhancing transport of a deubiquitinase inhibitor to a tumor cell or inflammed tissue in a mammal comprising administering to the mammal a pharmaceutical composition comprising a deubiquitinase inhibitor and albumin.

In yet another aspect, described herein is a method of enhancing binding of a deubiquitinase inhibitor to a tumor cell in a mammal comprising administering to the mammal a pharmaceutical composition comprising a deubiquitinase inhibitor and albumin.

In yet another aspect, described herein is a method of increasing the accumulation of a deubiquitinase inhibitor in a tumor or inflamed tissue of a mammal comprising administering to the mammal a pharmaceutical composition comprising a deubiquitinase inhibitor and albumin.

In yet another aspect, described herein is a method of improving the pharmacokinetic profile of a deubiquitinase inhibitor comprising administering to the mammal a pharmaceutical composition comprising a deubiquitinase inhibitor and albumin

In some embodiments, the pharmaceutical composition is administered to the mammal intravenously or subcutaneously.

In some embodiments, the albumin is human serum albumin.

In some embodiments, the deubiquitinase inhibitor is a small molecule compound.

In some embodiments, the deubiquitinase inhibitor is a small molecule cyano-substituted acrylamide compound.

In some embodiments, the deubiquitinase inhibitor has the following structure:

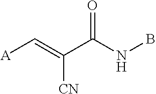

wherein,
A is a substituted or unsubstituted heteroaryl or substituted or unsubstituted aryl; and
B is a substituted or unsubstituted -(alkylene)-aryl or a substituted or unsubstituted -(alkylene)-heteroaryl.

In some embodiments, the deubiquitinase inhibitor has the structure of Formula (I), or a pharmaceutically acceptable salt, or solvate thereof:

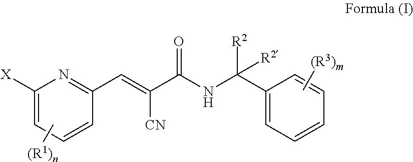

wherein,
X is H, F, Cl, Br, —OH, alkyl, —O-alkyl, —S-alkyl, —NH₂ or —NH-alkyl;
R¹ is H, halogen, CN, NO₂, unsubstituted or substituted alkyl, unsubstituted or substituted cycloalkyl, —O-(unsubstituted or substituted alkyl), —S-(unsubstituted or substituted alkyl), or —NH-(unsubstituted or substituted alkyl);
or R¹ and X are taken together with the intervening atoms connecting R¹ and X to form a heterocyclic ring with 1 or 2 heteroatoms selected from O, N, and S;
R² is H, unsubstituted or substituted alkyl, unsubstituted or substituted heteroalkyl, unsubstituted or substituted cycloalkyl, unsubstituted or substituted heterocycloalkyl, or unsubstituted or substituted benzyl;
or R² is -L¹-R⁶ where,
   L¹ is bond, unsubstituted or substituted alkyl, unsubstituted or substituted cycloalkyl, or unsubstituted or substituted heteroalkyl;
   R⁶ is unsubstituted or substituted cycloalkyl, unsubstituted or substituted heterocycloalkyl, unsubstituted or substituted aryl, or unsubstituted or substituted heteroaryl;
R^{2′} is H or unsubstituted or substituted alkyl;
each R³ is independently selected from H, halogen, —CN, —NO₂, —OH, —OR⁴, —SR⁴, —S(═O)R⁴, —S(═O)₂R⁴, —S(═O)₂N(R⁵)₂, —NR⁵S(═O)₂R⁴, —C(═O)R⁴, —OC(═O)R⁴, —CO₂R⁵, —OCO₂R⁴, —N(R⁵)₂, —C(═O)N(R⁵)₂, —OC(═O)N(R⁵)₂, —NHC(═O)R⁴, —NHC(═O)OR⁴, unsubstituted or substituted alkyl, unsubstituted or substituted alkenyl, unsubstituted or substituted alkynyl, unsubstituted or substituted heteroalkyl, unsubstituted or substituted cycloalkyl, unsubstituted or substituted aryl, unsubstituted or substituted heteroaryl, and -L²-L³-L⁴-L⁵-R⁷;
   L² is absent, —O—, —S—, —S(O)—, —S(O)₂₋, —NR⁴—, —CH(OH)—, —C(═O)—, —C(═O)NH—, —NHC(═O)—, —C(═O)O—, —OC(═O)—, —CH(═N)—, —CH(═N—NH)—, —CCH₃(═N)—, —CCH₃(═N—NH)—, —OC(═O)NH—, —NHC(═O)NH—, —NHC(═O)O—, —(CH₂)ₚ—, or —(OCH₂CH₂)ₚ—, p is 1, 2, 3, or 4;
   L³ is absent, unsubstituted or substituted alkylene, unsubstituted or substituted heteroalkylene, unsubstituted or substituted alkenylene, unsubstituted or substituted alkynylene, unsubstituted or substituted cycloalkylene, unsubstituted or substituted heterocycloalkylene, unsubstituted or substituted arylene, unsubstituted or substituted heteroarylene, or —(OCH₂CH₂)ₚ—, p is 1, 2, 3, or 4;
   L⁴ is absent, —O—, —S—, —S(O)—, —S(O)₂₋, —NR⁴—, —CH(OH)—, —C(═O)—, —C(═O)NH—, —NHC(═O)—, —C(═O)O—, —OC(═O)—, —OC(═O)NH—, —NHC(═O)NH—, or —NHC(═O)O—;
   L⁵ is absent, unsubstituted or substituted alkylene, unsubstituted or substituted heteroalkylene;
   R⁷ is H, halogen, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, or unsubstituted or substituted heteroaryl;
R⁴ is C₁-C₆alkyl, C₁-C₆fluoroalkyl, C₁-C₆deuteroalkyl, C₃-C₆cycloalkyl, a substituted or unsubstituted phenyl, a substituted or unsubstituted monocyclic heteroaryl, or a substituted or unsubstituted bicyclic heteroaryl;
each R⁵ is independently selected from H, C₁-C₆alkyl, C₁-C₆fluoroalkyl, C₁-C₆deuteroalkyl, C₃-C₆cycloalkyl, a substituted or unsubstituted phenyl, or a substituted or unsubstituted monocyclic heteroaryl; or
two R⁵ groups attached to the same N atom are taken together with the N atom to which they are attached to form a substituted or unsubstituted heterocycle
m is 0, 1, 2, 3, or 4;
n is 0, 1, 2 or 3.

In some embodiments, the deubiquitinase inhibitor is a compound as described in Table 1, or a pharmaceutically acceptable salt, or solvate thereof.

In some embodiments, the deubiquitinase inhibitor is physically bound to the albumin through non-covalent interactions.

In some embodiments, the deubiquitinase inhibitor and albumin are formulated as particles.

In some embodiments, particles of the deubiquitinase inhibitor are coated with the albumin.

In some embodiments, the ratio of the deubiquitinase inhibitor to albumin is from about 1:20 to about 20:1.

In some embodiments, the deubiquitinase inhibitor is covalently conjugated with an albumin binding group.

In some embodiments, the deubiquitinase inhibitor is covalently conjugated with an albumin binding group with or without a linker as depicted with the following Formula B:

(DUB)-L-Ab Formula B

wherein,
DUB is a deubiquitinase inhibitor;
L is a absent or a linker;
Ab is an albumin binding group.

In some embodiments, the deubiquitinase inhibitor is a small molecule compound.

In some embodiments, the deubiquitinase inhibitor is a small molecule cyano-substituted acrylamide compound.

In some embodiments, the deubiquitinase inhibitor has the following structure:

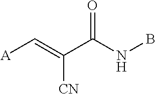

wherein,
A is a substituted or unsubstituted heteroaryl or substituted or unsubstituted aryl; and
B is a substituted or unsubstituted -(alkylene)-aryl or a substituted or unsubstituted -(alkylene)-heteroaryl.

In some embodiments, the albumin binding group increases the binding affinity of the deubiquitinase inhibitor to human serum albumin.

In some embodiments, the albumin binding group is a fatty acid, steroid, or thyroid hormone.

In some embodiments, the albumin binding group is a saturated alkyl acid or an unsaturated alkyl acid.

In some embodiments, the albumin binding group is a butanoic acid (butyric acid), pentanoic acid (valeric acid), hexanoic acid (caproic acid), heptanoic acid (enanthic acid), octanoic acid (caprylic acid), nonanoic acid (pelargonic acid), decanoic acid (capric acid), undecanoic acid (undecylic acid), dodecanoic acid (lauric acid), tridecanoic acid (tridecylic acid), tetradecanoic acid (myristic acid), pentadecanoic acid (pentadecylic acid), hexadecanoic acid (palmitic acid), heptadecanoic acid (margaric acid), octadecanoic acid (stearic acid), nonadecanoic acid (nonadecylic acid), eicosanoic acid (arachidic acid), heneicosanoic acid, docosanoic acid (behenic acid), tricosanoic acid, tetracosanoic acid (lignoceric acid), pentacosanoic acid, hexaconsanoic acid (cerotic acid), heptacosanoic acid, octacosanoic acid (montanic acid), nonacosanoic acid, triacontanoic acid (melissic acid), or hentriacontanoic acid.

In some embodiments, the albumin binding group is myristic acid.

In some embodiments, the albumin binding group is a saturated alkyl acid that is covalently conjugated to the deubiquitinase inhibitor as depicted with the following Formula B-1:

(DUB)-L-X—Y-(Alk) Formula B-1

wherein,
DUB is a deubiquitinase inhibitor;
L is a absent or a linker;
X is absent, —O—, —S— or —NH—;
Y is —C(═O)—;
Alk is C₆-C₄₀alkyl.

In some embodiments, L is a metabolizable linker.

In some embodiments, L is —CH═N—, —C(alkyl)=N—, —CH═N—NH—, —C(alkyl)=N—NH—, or —S—S—.

In some embodiments, Alk is —(CH₂)₅—CH₃, —(CH₂)₆—CH₃, —(CH₂)₇—CH₃, —(CH₂)₈—CH₃, —(CH₂)₉—CH₃, —(CH₂)₁₀—CH₃, —(CH₂)₁₁—CH₃, —(CH₂)₁₂—CH₃, —(CH₂)₁₃—CH₃, —(CH₂)₁₄—CH₃, —(CH₂)₁₅—CH₃, —(CH₂)₁₆—CH₃, —(CH₂)₁₇—CH₃, —(CH₂)₁₈—CH₃, —(CH₂)₁₉—CH₃, —(CH₂)₂₀—CH₃, —(CH₂)₂₁—CH₃, —(CH₂)₂₂—CH₃, —(CH₂)₂₃—CH₃, —(CH₂)₂₄—CH₃, —(CH₂)₂₅—CH₃, —(CH₂)₂₆—CH₃, —(CH₂)₂₇—CH₃, —(CH₂)₂₈—CH₃, —(CH₂)₂₉—CH₃, —(CH₂)₃₀—CH₃, —(CH₂)₃₁—CH₃, —(CH₂)₃₂—CH₃, —(CH₂)₃₃—CH₃, —(CH₂)₃₄—CH₃, —(CH₂)₃₅—CH₃, —(CH₂)₃₆—CH₃, —(CH₂)₃₇—CH₃, —(CH₂)₃₈—CH₃, or —(CH₂)₃₉—CH₃.

In some embodiments, Alk is —(CH₂)₁₂—CH₃.

In some embodiments, the albumin binding group is capable of covalently binding to human serum albumin.

In some embodiments, the albumin binding group is capable of covalently binding to the free thiol (Cys 34) of human serum albumin.

In some embodiments, the albumin binding group is capable of covalently binding to a free amino of any one of the lysine residues of human serum albumin.

In some embodiments, the albumin binding group comprises a maleimide group, a halogenacetamide group, a halogenacetyl group, a halogenacetate group, a pyrdinylthio group, a vinylcarbonyl group, an aziridinyl group, a disulfide group, an acetylene group, a hydroxysuccinimide group or a thiol group.

In some embodiments, the albumin binding group comprises a maleimide group:

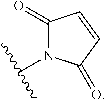 [SMILES: *N1C(=O)C=CC1=O]

In some embodiments, L is absent, alkylene, alkylene acid, a polyethylene glycol, polyethylene glycol acid, polyethylene glycol diacid, amino polyethylene glycol, amino polyethylene glycol acid, amino polyethylene glycol diacid or polyglycine.

In some embodiments, L is absent,

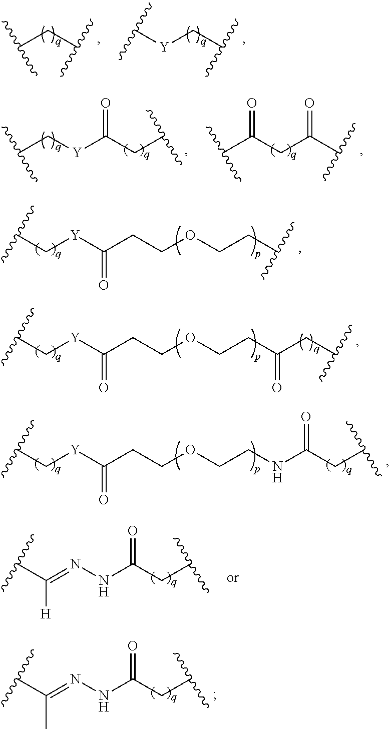

wherein Y is O or NH; each q is independently selected from 0, 1, 2, 3, 4, and 5; and p is 0, 1, 2, 3 or 4.

In some embodiments, each q is independently selected from 0, 1, 2, 3, and 4. In some embodiments, q is 0, 1, 2, 3, or 4.

In some embodiments, -L-Ab is:

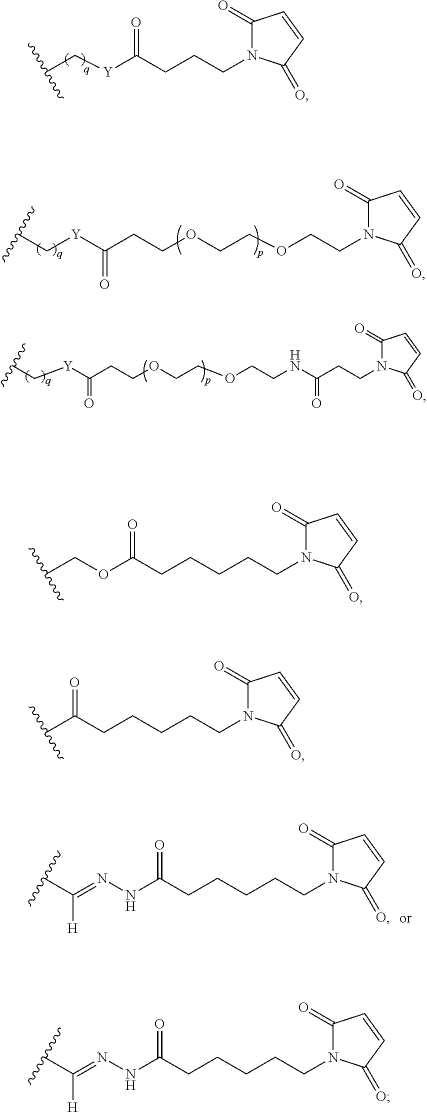

wherein Y is O or NH; q is 0, 1, 2 or 3; and p is 0, 1, 2 or 3.

In one aspect, described herein is a pharmaceutical composition comprising a deubiquitinase inhibitor and albumin. In some embodiments, the albumin is human serum albumin. In some embodiments, the ratio of the deubiquitinase inhibitor to albumin is from about 1:20 to about 20:1. In some embodiments, the pharmaceutical composition is formulated for intravenous or subcutaneous injection to a mammal. In some embodiments, the deubiquitinase inhibitor is a small molecule compound. In some embodiments, the deubiquitinase inhibitor is a small molecule cyano-substituted acrylamide compound.

In some embodiments, the deubiquitinase inhibitor has the following structure:

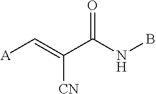

wherein,
A is a substituted or unsubstituted heteroaryl or substituted or unsubstituted aryl; and
B is a substituted or unsubstituted -(alkylene)-aryl or a substituted or unsubstituted -(alkylene)-heteroaryl.

In some embodiments, the deubiquitinase inhibitor has the structure of Formula (I), or a pharmaceutically acceptable salt, or solvate thereof:

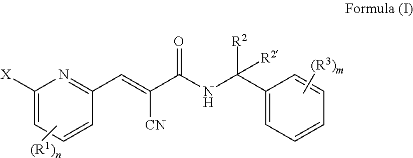

wherein,
X is H, F, Cl, Br, —OH, alkyl, —O-alkyl, —S-alkyl, —NH₂ or —NH-alkyl;
R¹ is H, halogen, CN, NO₂, unsubstituted or substituted alkyl, unsubstituted or substituted cycloalkyl, —O-(unsubstituted or substituted alkyl), —S-(unsubstituted or substituted alkyl), or —NH-(unsubstituted or substituted alkyl);
or R¹ and X are taken together with the intervening atoms connecting R¹ and X to form a heterocyclic ring with 1 or 2 heteroatoms selected from O, N, and S;
R² is H, unsubstituted or substituted alkyl, unsubstituted or substituted heteroalkyl, unsubstituted or substituted cycloalkyl, unsubstituted or substituted heterocycloalkyl, or unsubstituted or substituted benzyl;
or R² is -L¹-R⁶ where,
L¹ is bond, unsubstituted or substituted alkyl, unsubstituted or substituted cycloalkyl, or unsubstituted or substituted heteroalkyl;
R⁶ is unsubstituted or substituted cycloalkyl, unsubstituted or substituted heterocycloalkyl, unsubstituted or substituted aryl, or unsubstituted or substituted heteroaryl;
R^{2′} is H or unsubstituted or substituted alkyl;
each R³ is independently selected from H, halogen, —CN, —NO₂, —OH, —OR⁴, —SR⁴, —S(═O)R⁴, —S(═O)₂R⁴, —S(═O)₂N(R⁵)₂, —NR⁵S(═O)₂R⁴, —C(═O)R⁴, —OC(═O)R⁴, —CO₂R⁵, —OCO₂R⁴, —N(R⁵)₂, —C(═O)N(R⁵)₂, —OC(═O)N(R⁵)₂, —NHC(═O)R⁴, —NHC(═O)OR⁴, unsubstituted or substituted alkyl, unsubstituted or substituted alkenyl, unsubstituted or substituted alkynyl, unsubstituted or substituted heteroalkyl, unsubstituted or substituted cycloalkyl, unsubstituted or substituted aryl, unsubstituted or substituted heteroaryl, and -L²-L³-L⁴-L⁵-R⁷;
   L² is absent, —O—, —S—, —S(O)—, —S(O)₂₋, —NR⁴—, —CH(OH)—, —C(═O)—, —C(═O)NH—, —NHC(═O)—, —C(═O)O—, —OC(═O)—, —CH(═N)—, —CH(═N—NH)—, —CCH₃(═N)—, —CCH₃(═N—NH)—, —OC(═O)NH—, —NHC(═O)NH—, —NHC(═O)O—, —(CH₂)ₚ—, or —(OCH₂CH₂)ₙ—, p is 1, 2, 3, or 4;
   L³ is absent, unsubstituted or substituted alkylene, unsubstituted or substituted heteroalkylene, unsubstituted or substituted alkenylene, unsubstituted or substituted alkynylene, unsubstituted or substituted cycloalkylene, unsubstituted or substituted heterocycloalkylene, unsubstituted or substituted arylene, unsubstituted or substituted heteroarylene, or —(OCH₂CH₂)ₚ—, p is 1, 2, 3, or 4;
   L⁴ is absent, —O—, —S—, —S(O)—, —S(O)₂₋, —NR⁴—, —CH(OH)—, —C(═O)—, —C(═O)NH—, —NHC(═O)—, —C(═O)O—, —OC(═O)—, —OC(═O)NH—, —NHC(═O)NH—, or —NHC(═O)O—;
   L⁵ is absent, unsubstituted or substituted alkylene, unsubstituted or substituted heteroalkylene;
   R⁷ is H, halogen, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, or unsubstituted or substituted heteroaryl;
R⁴ is C₁-C₆alkyl, C₁-C₆fluoroalkyl, C₁-C₆deuteroalkyl, C₃-C₆cycloalkyl, a substituted or unsubstituted phenyl, a substituted or unsubstituted monocyclic heteroaryl, or a substituted or unsubstituted bicyclic heteroaryl;
each R⁵ is independently selected from H, C₁-C₆alkyl, C₁-C₆fluoroalkyl, C₁-C₆deuteroalkyl, C₃-C₆cycloalkyl, a substituted or unsubstituted phenyl, or a substituted or unsubstituted monocyclic heteroaryl; or
two R⁵ groups attached to the same N atom are taken together with the N atom to which they are attached to form a substituted or unsubstituted heterocycle
m is 0, 1, 2, 3, or 4;
n is 0, 1, 2, or 3.

In some embodiments, the deubiquitinase inhibitor is a compound as described in Table 1, or a pharmaceutically acceptable salt, or solvate thereof.

In some embodiments, the deubiquitinase inhibitor is physically bound to the albumin through non-covalent interactions.

In some embodiments, the pharmaceutical composition comprises albumin coated particles of the deubiquitinase inhibitor.

In some embodiments, the deubiquitinase inhibitor is covalently conjugated with an albumin binding group.

In some embodiments, the deubiquitinase inhibitor is covalently conjugated with an albumin binding group with or without a linker as depicted with the following Formula B:

(DUB)-L-Ab Formula B

wherein,
DUB is a deubiquitinase inhibitor;
L is a absent or a linker;
Ab is an albumin binding group.

In some embodiments, the deubiquitinase inhibitor is a small molecule compound.

In some embodiments, the deubiquitinase inhibitor is a small molecule cyano-substituted acrylamide compound.

In some embodiments, the deubiquitinase inhibitor has the following structure:

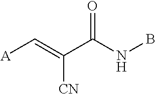

wherein,
A is a substituted or unsubstituted heteroaryl or substituted or unsubstituted aryl; and
B is a substituted or unsubstituted -(alkylene)-aryl or a substituted or unsubstituted -(alkylene)-heteroaryl.

In some embodiments, the albumin binding group increases the binding affinity of the deubiquitinase inhibitor to human serum albumin.

In some embodiments, the albumin binding group is a fatty acid, steroid, or thyroid hormone.

In some embodiments, the albumin binding group is a saturated alkyl acid or an unsaturated alkyl acid.

In some embodiments, the albumin binding group is a butanoic acid (butyric acid), pentanoic acid (valeric acid), hexanoic acid (caproic acid), heptanoic acid (enanthic acid), octanoic acid (caprylic acid), nonanoic acid (pelargonic acid), decanoic acid (capric acid), undecanoic acid (undecylic acid), dodecanoic acid (lauric acid), tridecanoic acid (tridecylic acid), tetradecanoic acid (myristic acid), pentadecanoic acid (pentadecylic acid), hexadecanoic acid (palmitic acid), heptadecanoic acid (margaric acid), octadecanoic acid (stearic acid), nonadecanoic acid (nonadecylic acid), eicosanoic acid (arachidic acid), heneicosanoic acid, docosanoic acid (behenic acid), tricosanoic acid, tetracosanoic acid (lignoceric acid), pentacosanoic acid, hexaconsanoic acid (cerotic acid), heptacosanoic acid, octacosanoic acid (montanic acid), nonacosanoic acid, triacontanoic acid (melissic acid), or hentriacontanoic acid.

In some embodiments, the albumin binding group is myristic acid.

In some embodiments, the albumin binding group is a saturated alkyl acid that is covalently conjugated to the deubiquitinase inhibitor as depicted with the following Formula B-1:

(DUB)-L-X—Y-(Alk) Formula B-1

wherein,
DUB is a deubiquitinase inhibitor described herein;
L is a absent or a linker;
X is absent, —O—, —S— or —NH—;
Y is —C(═O)—;
Alk is C₆-C₄₀alkyl.

In some embodiments, L is a metabolizable linker.

In some embodiments, L is —CH═N—, —C(alkyl)=N—, —CH═N—NH—, —C(alkyl)=N—NH—, or —S—S—.

In some embodiments, Alk is —(CH₂)₅—CH₃, —(CH₂)₆—CH₃, —(CH₂)₇—CH₃, —(CH₂)₈—CH₃, —(CH₂)₉—CH₃, —(CH₂)₁₀—CH₃, —(CH₂)₁₁—CH₃, —(CH₂)₁₂—CH₃, —(CH₂)₁₃—CH₃, —(CH₂)₁₄—CH₃, —(CH₂)₁₅—CH₃, —(CH₂)₁₆—CH₃, —(CH₂)₁₇—CH₃, —(CH₂)₁₈—CH₃, —(CH₂)₁₉—CH₃, —(CH₂)₂₀—CH₃, —(CH₂)₂₁—CH₃, —(CH₂)₂₂—CH₃, —(CH₂)₂₃—CH₃, —(CH₂)₂₄—CH₃, —(CH₂)₂₅—CH₃, —(CH₂)₂₆—CH₃, —(CH₂)₂₇—CH₃, —(CH₂)₂₈—CH₃, —(CH₂)₂₉—CH₃, —(CH₂)₃₀—CH₃, —(CH₂)₃₁—CH₃, —(CH₂)₃₂—CH₃, —(CH₂)₃₃—CH₃, —(CH₂)₃₄—CH₃, —(CH₂)₃₅—CH₃, —(CH₂)₃₆—CH₃, —(CH₂)₃₇—CH₃, —(CH₂)₃₈—CH₃, or —(CH₂)₃₉—CH₃.

In some embodiments, Alk is —(CH₂)₁₂—CH₃.

In some embodiments, the albumin binding group is capable of covalently binding to human serum albumin.

In some embodiments, the albumin binding group is capable of covalently binding to the free thiol (Cys 34) or free —NH₂ (lysine(s)) of human serum albumin.

In some embodiments, the albumin binding group comprises a maleimide group, a halogenacetamide group, a halogenacetyl group, a halogenacetate group, a pyrdinylthio group, a vinylcarbonyl group, an aziridinyl group, a disulfide group, an acetylene group, a hydroxysuccinimide group or a thiol group.

In some embodiments, the albumin binding group comprises a maleimide group:

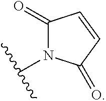 [SMILES: *N1C(=O)C=CC1=O]

In some embodiments, L is absent, alkylene, alkylene acid, a polyethylene glycol, polyethylene glycol acid, polyethylene glycol diacid, amino polyethylene glycol, amino polyethylene glycol acid, amino polyethylene glycol diacid or polyglycine.

In some embodiments, L is absent,

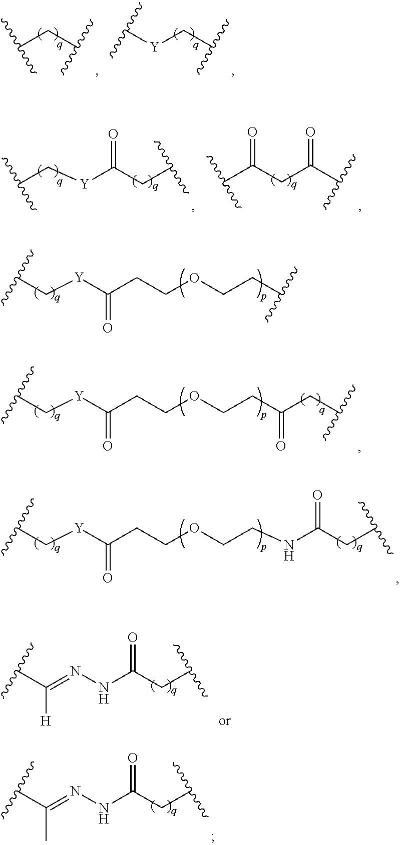

wherein Y is O or NH; each q is independently selected from 0, 1, 2, 3, 4, and 5; and p is 0, 1, 2, 3 or 4.

In some embodiments, each q is independently selected from 0, 1, 2, 3, and 4. In some embodiments, q is 0, 1, 2, 3, or 4.

In some embodiments, -L-Ab is:

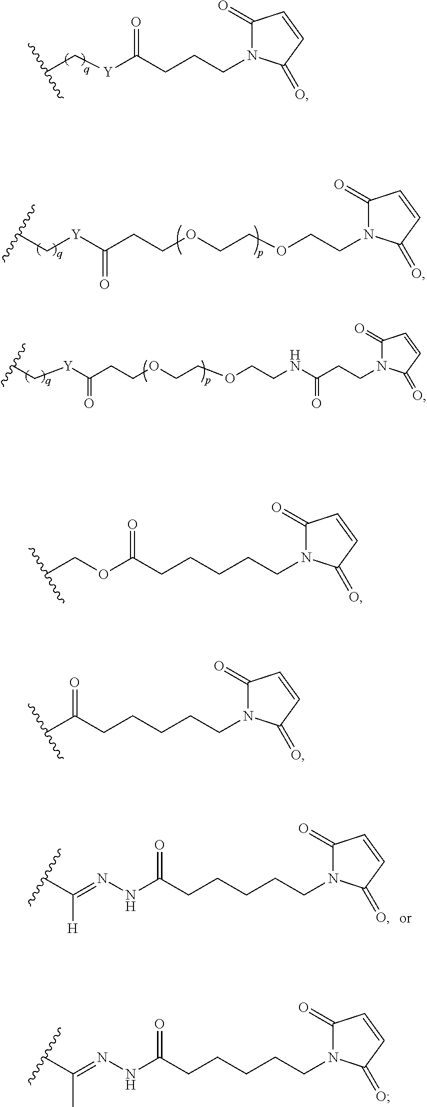

wherein Y is O or NH; q is 0, 1, 2 or 3; and p is 0, 1, 2 or 3.

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### DETAILED DESCRIPTION OF THE INVENTION

Ubiquitin is a 76 amino acid polypeptide that is covalently attached to proteins through the ubiquitination machinery. This machinery consists of an ubiquitin-activating enzyme (E1) that forms a thiol-ester intermediate with the C-terminal glycine residue of a ubiquitin monomer, a ubiquitin-conjugating enzyme (E2) to which the activated ubiquitin is transferred, and a ubiquitin ligase (E3) in which the final transfer of ubiquitin to a lysine residue of a protein substrate occurs. Ubiquitin addition can occur as a single molecule attachment to one or more lysine residues of the protein substrate (monoubiquitination) or as an ubiquitin chain (polyubiquitination). Ubiquitin contains seven internal lysine residues (K6, K11, K27, K29, K33, K48 and K63) and the ubiquitin chain can exist in several formats depending on the internal lysine residues utilized in the inter-ubiquitin linkage. These differently formatted ubiquitin chains further dictate the cellular fate of the ubiquitinated proteins. For example, ubiquitinated proteins containing the K48-linked chains are delivered to the 26S proteasome for degradation whereas ubiquitinated proteins containing the K63-linked chains alter target protein structure, localization and activity.

Ubiquitination is a reversible process and ubiquitin removal is tightly regulated by deubiquitinating enzymes. Deubiquitinating enzymes (DUBs) comprise a protease superfamily which mediates the removal of ubiquitin through a specific cleavage of isopeptide bonds at the C-terminus of ubiquitins. The human genome encodes ˜98 DUBs which are often part of large multi-protein complexes and are involved in several points along the ubiquitin pathway. For example, DUBs can process ubiquitins from polyubiquitin precursors, remove non-degradative ubiquitin signals, prevent protein degradation from the 26S proteasome and lysosomal pathways, maintain ubiquitin homeostasis and edit ubiquitin chains thereby modulating ubiquitin signals. DUBs activity can enhance protein stability by preventing protein degradation and the dysregulation in the activity and expression of DUBs has been linked to multiple diseases.

DUB activities fall into three major functional categories. First, ubiquitin can be transcribed from several genes as a linear fusion of multiple ubiquitin molecules or with ribosomal proteins, such that the generation of free ubiquitin requires DUB activity. Second, DUBs can remove ubiquitin chains from post-translationally modified proteins, leading to reversal of ubiquitin signaling or to protein stabilization by rescue from either proteasomal (for example, cytosolic proteins) or lysosomal (for example, internalized receptors) degradation. However, once a commitment to these degradative machines has been made, associated DUB activities can recycle ubiquitin, thereby contributing to ubiquitin homeostasis. Third, DUBs can be used to edit the form of ubiquitin modification by trimming ubiquitin chains.

The process of ubiquitination-deubiquitination modulates a myriad of cellular processes including regulation of gene expression, cell-cycle progression, modifications of cell surface receptors, biogenesis of ribosomes, chromosomal segregation, protein degradation, DNA damage responses, apoptosis and signal transduction pathways. These cellular processes are particularly relevant in several disease states such as cancer, fibrosis, neurodegenerative disorders, autoimmune dysfunction and pathogenic infections. For example, the transforming growth factor beta (TGF-β) signaling pathway is a centralized command center for cell growth, cell differentiation, apoptosis and cellular homeostasis, and its disruption gives rise to neurodegenerative and fibrotic diseases, autoimmune dysfunction and cancer. Multiple deubiquitinases have been implicated in the regulation of the TGF-β pathway, including UCHL5 (also known as UCH37), USP4, USP9X, USP11 and USP15. Furthermore, mutations in several DUBs have been linked to diseases ranging from cancer to neurological disorders.

### Deubiquitinating Enzymes (DUBs)

DUBs are classified into five main families: ubiquitin-specific proteases (USPs), ubiquitin C-terminal hydrolases (UCHs), ovarian tumor proteases (OTUs), Machado-Joseph disease protein domain proteases (or Josephin domain) and JAB1/MPN/MOV34 metalloenzymes (JAMMs). The USP, UCH, OTU and Machado-Joseph disease protein domain proteases are characterized as cysteine proteases and the JAMM proteins are characterized as Zn²⁺-containing metalloproteases.

The USP family includes USP1, USP2, USP3, USP4, USP5, USP6, USP7, USP8, USP9X, USP9Y, USP10, USP11, USP12, USP13, USP14, USP15, USP16, USP17 (DUB3), USP18, USP19, USP20, USP21, USP22, USP24, USP25, USP26, USP27X, USP28, USP29, USP30, USP31, USP32, USP33, USP34, USP35, USP36, USP37, USP38, USP39, USP40, USP41, USP42, USP43, USP44, USP45, USP46, USP47, USP48, USP49, USP50, USP51, USP52, USP53, USP54, CYLD, USPL1, and TL132.

The UCH family includes UCHL1, UCHL3, UCHL5, and BAP1.

The OUT family includes OTUB1, OTUB2, OTUD1, OTUD3, OTUD4, HIN1L, OTUD5, OTUD6A, OTUD6B, OTU1, A20, Cezanne-1, Cezanne-2, TRABID and VCPIP1.

The Machado-Joseph disease protein domain protease family includes ATXN3, ATXN3L, JOSD1 and JOSD2. The JAMM/MPN+ family includes BRCC36, CSN5, POH1, AMSH, AMSH-LP, MPND, PSMD7, MYSM1, PRPF8, EIF3S3 and EIF3S5. Specific members of the DUB superfamily, for example USP and UCH family members such as USP9X, USP14, USP5, USP7, USP24, UCHL1 and UCHL5, have been linked to many diseases.

### USP9X

USP9X (or FAM) is associated with a variety of substrates such as SMAD4, MCL1, β-catenin, α-Synuclein, AF-6 and EFA6 which have been implicated in cancer and neurodegenerative diseases. For example, SMAD4 is a central transducer of TGFβ responses and interacts with various members of the R-SMADs forming heteromeric complexes which regulate gene expression. SMAD4 is the only coactivating SMAD (Co-SMAD) mediating signaling downstream of the TGFβ and BMP signaling pathways and its nuclear localization and activation are controlled by USP9x mediated deubiquitination (see Dupont et al, “FAM/USP9x, a deubiquitinating enzyme essential for TGF-β signaling, controls Smad4 monoubiquitination” Cell 136:123-135 (2009). Another critical protein for survival of stem and progenitor cells of multiple lineages is the pro-survival protein MCL1. It has been observed that down regulation or pharmacological inhibition of USP9x inhibits tumor cell growth by promoting degradation of MCL1 (see Schwickart et al., “Deubiquitinase USP9x stabilizes MCL1 and promotes tumour cell survival” Nature 463:103-107 (2010). In addition, USP9X has been observed to be upregulated in myeloma, breast, pancreatic, colon and prostate cancer cells.

The transcription factor E-twenty-six related gene (ERG) is overexpressed through gene fusion in ˜40% of prostate tumors and is a key driver of prostate cancer. USP9x binds ERG and deubiquitinates it. Pharmacological inhibition of USP9x results in the degradation of ERG and attenuates the growth of ERG positive prostate tumor cells in vitro and in vivo (see Wang et al., “Ablation of the oncogenic transcription factor ERG by deubiquitinase inhibition in prostate cancer” Proc Nat Acad Sci USA 111(11):4251-4256, 2014).

SMURF1 is an E3 ligase specific for receptor-regulated SMAD proteins and plays a role in cancer cell migration and invasion. Indeed, in MDA-MB-231 breast cancer cells, SMURF1 expression is elevated and depletion of USP9X leads to down-regulation of SMURF1 and impaired cellular migration (see, Xie, et al., “Deubiquitinase FAM/USP9X interact with the E3 ubiquitin ligase SMURF1 and protects it from ligase activity-dependent self-degradation,” J. Biol. Chem. 288(5):2976-2985 (2013)).

### USP24

USP24 is highly homologous to USP9x and in some cells has been localized in the nucleus where it plays a role in DNA repair by stabilizing DNA damage-binding protein 2 (Zhang et al., “The deubiquitinating protein USP24 interacts with DDB2 and regulates DDB2 stability” Cell Cycle 11:4378-4384, 2012). In addition, USP24 gene transcription is tightly regulated by NFKβ, a protein which has significant roles in inflammation and apoptosis (see Wang et al., “Transcriptional regulation of human USP24 gene expression by NFKβ”. J Neurochem 128(6):818-828 (2014)).

### USP7

USP7 (or HAUSP) is shown to interact with both viral and cellular proteins. In one aspect, USP7 interacts with PTEN, an antagonist in the AKT signaling pathway responsible for glucose metabolism, apoptosis, cell proliferation, transcription and cell migration. Reduced nuclear localization of PTEN is a common feature in aggressive cancers such as bladder, prostate, colon, liver, lung and prostate cancers. Indeed, overexpression of USP7 inhibits the nuclear localization of PTEN in prostate cancer cells (Song, et al., “The deubiquitinylation and localization of PTEN are regulated by a HAUSP-PML network,” Nature 455:813-817, 2008). In addition, inhibition of USP7 has been shown to induce apoptosis in multiple myeloma cells and to overcome cell resistance to chemotherapeutic agents such as bortezomib. Further, USP7 interacts with Vmw110, a viral protein that is required for the lytic cycle of herpes simplex virus, and with EBNA1, encoded by the Epstein-Barr virus, in which EBNA1 disrupts USP7's interaction with p53.

### USP14

USP14 is shown to be highly expressed in several cancer cell lines including multiple myeloma cells and colorectal carcinoma cells. Further, USP14 mediates the deubiquitination of Dishevelled (Dvl), a key regulator of the Wnt signaling pathway which shows abberant activation in diverse cancers including colorectal cancer (Jung, et al., “Deubiquitination of Dishevelled by Usp14 is required for Wnt signaling,” Oncogenesis, 2(8):e64 (2013).

### USP5

USP5 (or isopeptidase T, ISOT) modulates the p53 pathway by preferentially processing unanchored polyubiquitin into free ubiquitins. The unanchored polyubiquitins compete with ubiquitinated p53 for proteasomal recognition and degradation to free ubiquitins. In one aspect, the inhibition of USP5 leads to the activation and stabilization of p53 by interfering with the degradation of ubiquitinated p53. (See, Dayal, et al, “Suppression of the deubiquitinating enzyme USP5 causes the accumulation of unanchored polyubiquitin and the activation of p53,” J. Biol. Chem. 284(8):5030-5041 (2009)

### UCHL1

UCHL1 (or PGP9.5) is upregulated in numerous cancers including lung, colorectal, pancreatic, thyroid, myeloma and B-lymphocyte cancers. For example, overexpression of UCHL1 is observed in 54% of all non-small-cell lung carcinomas and in 75% of stages II and III non-small-cell lung carcinomas. In colorectal cancer, 46% of the specimens expressed UCHL1. In addition, overexpression of UCHL1 has been correlated to increased tumor size and invasiveness in lung cancer patients and has been negatively correlated to postoperative survival in pancreatic cancer patients. Further, UCHL1 is shown to interact and colocalize with JAB1, a Jun activation domain-binding protein that interacts with p27. This interaction and colocalization may contribute to the degradation of p27, a cyclin-dependent kinase inhibitor whose loss in epithelial cancers has been correlated with pathological tumor grade where high-grade tumors exhibit significantly lower expression of p27 than their counterparts. The decreased expression of p27 has also been observed in breast, lung, PTEN prostate, colon, skin and ovarian cancers and has been correlated with cancer development and poor survival.

### UCHL5 (Also Known as UCH37)

UCHL5 interacts with glucose-regulated protein 78 which is essential for cell viability and can serve as a predictor of recurrence of hepatocellular carcinoma (see, Hirohashi, et al., “p78/MCRS1 forms a complex with centrosomal protein Nde1 and is essential for cell viability,” Oncogene 25:425-479, 2006). In some cases, UCHL5 can further serve as a predictor for overall survival (OS) and disease-free survival (DFS) in esophageal squamous cell carcinoma (ESCC) patients (see, Chen, et al., “Expression and clinical significance of UCH37 in human esophageal squamous cell carcinoma,” Dig Dis Sci 57:2310-2017, 2012).

### DUBs and DUB Inhibition

Disclosed herein are compositions and methods of inhibiting a deubiquitinating enzyme (DUB). In some embodiments, a compound described herein inhibits all members of the DUB superfamily. In some embodiments, a compound described herein inhibits one or more members of the DUB superfamily. In some embodiments, the DUBs include USP1, USP2, USP3, USP4, USP5, USP6, USP7, USP8, USP9X, USP9Y, USP10, USP11, USP12, USP13, USP14, USP15, USP16, USP17 (DUB3), USP18, USP19, USP20, USP21, USP22, USP24, USP25, USP26, USP27X, USP28, USP29, USP30, USP31, USP32, USP33, USP34, USP35, USP36, USP37, USP38, USP39, USP40, USP41, USP42, USP43, USP44, USP45, USP46, USP47, USP48, USP49, USP50, USP51, USP52, USP53, USP54, CYLD, USPL1, TL132, UCHL1, UCHL3, UCHL5, BAP1, UCH37, OTUB1, OTUB2, OTUD1, OTUD3, OTUD4, HIN1L, OTUD5, OTUD6A, OTUD6B, OTU1, A20, Cezanne1, Cezanne 2, TRABID, VCPIP1, ATXN3, ATXN3L, JOSD1, JOSD2, BRCC36, CSN5, POH1, AMSH, AMSH-LP, MPND, MYSM1, PRPF8, PSMD7, EIF3S3 and EIF3S5.

In some embodiments, a compound described herein inhibits any one of USP1, USP2, USP3, USP4, USP5, USP6, USP7, USP8, USP9X, USP9Y, USP10, USP11, USP12, USP13, USP14, USP15, USP16, USP17 (DUB3), USP18, USP19, USP20, USP21, USP22, USP24, USP25, USP26, USP27X, USP28, USP29, USP30, USP31, USP32, USP33, USP34, USP35, USP36, USP37, USP38, USP39, USP40, USP41, USP42, USP43, USP44, USP45, USP46, USP47, USP48, USP49, USP50, USP51, USP52, USP53, USP54, CYLD, USPL1, TL132, UCHL1, UCHL3, UCHL5, BAP1, UCH37, OTUB1, OTUB2, OTUD1, OTUD3, OTUD4, HIN1L, OTUD5, OTUD6A, OTUD6B, OTU1, A20, Cezanne1, Cezanne 2, TRABID, VCPIP1, ATXN3, ATXN3L, JOSD1, JOSD2, BRCC36, CSN5, POH1, AMSH, AMSH-LP, MPND, MYSM1, PRPF8, PSMD7, EIF3S3 and EIF3S5 or any combinations thereof.

In some embodiments, a compound described herein inhibits USP9X, USP14, USP5, USP24, or UCHL5, or any combinations thereof.

In some embodiments, the compound described herein inhibits a portion of a DUB. In some embodiments, the portion of the DUB comprises one or more domains of the DUB. In some embodiments, the compound described herein inhibits a portion of a DUB that is a conserved portion among the members of the DUB. In some embodiments, the compound described herein inhibits a portion of a DUB that is not conserved among the members of the DUB. In some embodiments, the compound described herein inhibits a portion that is conserved among USP9X, USP14, USP5, USP24, UCHL3 and UCHL5.

### DUBs and Diseases

Disclosed herein is a method of treating a disease or condition that would benefit from inhibition of the activity of one or more DUBs comprising administering a compound described herein, or a pharmaceutically acceptable salt thereof, to a mammal in need thereof. In some embodiments, disclosed herein is a method of reducing the progression of a disease associated with DUB activity, reversing the progression of a disease associated with DUB activity, extending the period of remission of a disease associated with DUB activity, or eliminating a disease associated with DUB activity in a mammal in need thereof comprising administering to the mammal a compound described herein. In some embodiments, the method of treatment refers to stopping the progression of a disease, partial or complete elimination of a disease, reversing progression of a disease, stopping, reducing or reversing episodes of worsening or relapses of a disease, or prolonging episodes of remission of a disease in a subject. In some embodiments, the disease is selected from a cancer, a fibrosis, an autoimmune disease or condition, a neurodegenerative disease or condition, or an infection.

### Cancer

In some embodiments, described herein is a method of treating cancer in a mammal comprising inhibiting the activity of one or more deubiquitinase enzymes in the mammal. In some embodiments, the method comprises administering a compound described herein, or a pharmaceutically acceptable salt thereof, to the mammal with a cancer. In some embodiments, the cancer is a solid tumor. In some embodiments, the solid tumor is a sarcoma or carcinoma. In some embodiments, the solid tumor is a sarcoma. In some embodiments, the solid tumor is a carcinoma. In some embodiments, the sarcoma is selected from alveolar rhabdomyosarcoma; alveolar soft part sarcoma; ameloblastoma; angiosarcoma; chondrosarcoma; chordoma; clear cell sarcoma of soft tissue; dedifferentiated liposarcoma; desmoid; desmoplastic small round cell tumor; embryonal rhabdomyosarcoma; epithelioid fibrosarcoma; epithelioid hemangioendothelioma; epithelioid sarcoma; esthesioneuroblastoma; Ewing sarcoma; extrarenal rhabdoid tumor; extraskeletal myxoid chondrosarcoma; extraskeletal osteosarcoma; fibrosarcoma; giant cell tumor; hemangiopericytoma; infantile fibrosarcoma; inflammatory myofibroblastic tumor; Kaposi sarcoma; leiomyosarcoma of bone; liposarcoma; liposarcoma of bone; malignant fibrous histiocytoma (MFH); malignant fibrous histiocytoma (MFH) of bone; malignant mesenchymoma; malignant peripheral nerve sheath tumor; mesenchymal chondrosarcoma; myxofibrosarcoma; myxoid liposarcoma; myxoinflammatory fibroblastic sarcoma; neoplasms with perivascular epitheioid cell differentiation; osteosarcoma; parosteal osteosarcoma; neoplasm with perivascular epitheioid cell differentiation; periosteal osteosarcoma; pleomorphic liposarcoma; pleomorphic rhabdomyosarcoma; PNET/extraskeletal Ewing tumor; rhabdomyosarcoma; round cell liposarcoma; small cell osteosarcoma; solitary fibrous tumor; synovial sarcoma; telangiectatic osteosarcoma. In some embodiments, the carcinoma is selected from anal cancer; appendix cancer; AIDS-related cancer; bile duct cancer (e.g. cholangiocarcinoma); bladder cancer; bone tumor; brain tumor; breast cancer; cervical cancer; colon cancer; cancer of Unknown Primary (CUP); esophageal cancer; eye cancer; fallopian tube cancer; gastroenterological cancer; head and neck cancer; kidney cancer; liver cancer; lung cancer; medulloblastoma; melanoma; oral cancer; ovarian cancer; pancreatic cancer; parathyroid disease; penile cancer; pituitary tumor; prostate cancer; rectal cancer; skin cancer; stomach cancer; testicular cancer; throat cancer; thyroid cancer; uterine cancer; vaginal cancer; or vulvar cancer. In some embodiments, the tumor is a metastatic tumor. In some embodiments, the cancer is a metastatic cancer.

In some embodiments, the cancer is selected from breast, bone, brain, colorectal, kidney, prostate and skin cancers. In some embodiments, the cancer is breast cancer. In some embodiments, the cancer is bone cancer. In some embodiments, the cancer is brain cancer. In some embodiments, the cancer is colorectal cancer. In some embodiments, the cancer is kidney cancer. In some embodiments, the cancer is prostate cancer. In some embodiments, the cancer is skin cancer.

In some embodiments, the breast cancer is ductal carcinoma in situ (intraductal carcinoma), lobular carcinoma in situ, invasive (or infiltrating) ductal carcinoma, invasive (or infiltrating) lobular carcinoma, inflammatory breast cancer, triple-negative breast cancer, paget disease of the nipple, phyllodes tumor, angiosarcoma or invasive breast carcinoma. In some embodiments, the invasive breast carcinoma is further categorized into subtypes. In some embodiments, the subtypes include adenoid cystic (or adenocystic) carcinoma, low-grade adenosquamous carcinoma, medullary carcinoma, mucinous (or colloid) carcinoma, papillary carcinoma, tubular carcinoma, metaplastic carcinoma, micropapillary carcinoma or mixed carcinoma.

In some embodiments, the bone cancer is chordrosarcoma, fibrosarcoma, lymphoma, malignant fibrous histiocytoma, metastatic bone disease, myeloma, osteosarcoma (OS) or Ewing's sarcoma. In some embodiments, chondrosarcoma is further classified as chondrosarcoma not otherwise specified, juxtacortical chondrosarcoma, myxoid chondrosarcoma, mesenchymal chondrosarcoma, clear cell chondrosarcoma and dedifferentiated chondrosarcoma. In some embodiments, osteosarcomas is further classified as conventional osteosarcomas (e.g. osteoblastic, chondroblastic, fibroblastic, epithelioid, giant-cell rich, small cell or telangiectatic), cortex-associated osteosarcomas (e.g. parosteal, dedifferentiated parosteal, periosteal, high-grade surface, or intracortical), low-grade (central) osteosarcomas, osteoblastoma-like osteosarcomas, disease-associated osteosarcoma (e.g. osteosarcoma in Paget's disease, osteosarcoma in fibrous dysplasia, or osteosarcomas in Mazabraud's disease), multicentric osteosarcomas, post-irradiation osteosarcoma, or osteosarcoma of the gnathic bones.

In some embodiments, the brain tumor is adenoma, astrocytoma, atypical teratoid rhaboid tumor (ATRT), chondrosarcoma, chordomas, choroid plexus, craniopharyngioma, cysts, ependymoma, germ cell tumor, glioblastoma, glioma, hemangioma, lipoma, medulloblastoma, meningioma, metastatic brain tumor, neurofibroma, neuronal and mixed neuronal-glial tumors, oligoastrocytoma, oligodendroglioma, pineal tumors, pituitary tumors, PNET or schwannoma.

In some embodiments, the colorectal cancer is adenocarcinoma (e.g. mucinous adenocarcinoma, signet ring cell adenocarcinoma), gastrointestinal carcinoid tumor, gastrointestinal stromal tumor, primary colorectal lymphoma, leiomyosarcoma, melanoma or squamous cell carcinoma.

In some embodiments, the kidney cancer is renal cell carcinoma, renal pelvis carcinoma, squamous cell carcinoma, juxtaglomerular cell tumor, angiomyolipoma, renal oncocytoma, Bellini duct carcinoma, clear cell sarcoma of the kidney, mesoblastic nephroma, Wilms' tumor, mixed epithelial stromal tumor, clear cell adenocarcinoma, transitional cell carcinoma (urothelial cell carcinoma), inverted papilloma, renal lymphoma, teratoma, carcinosarcoma, carcinoid tumor off the renal pelvis or oncocytomas. In some embodiments, renal cell carcinoma is further categorized into chromophobe renal cell carcinoma (ChRCC), papillary renal cell carcinoma (PRCC), clear cell renal cell carcinoma (CCRCC), multilocular cystic renal cell carcinoma, carcinoma of the collecting ducts of Bellini, medullary carcinoma, Xp11.2 translocation carcinoma, mucinous tubular spindle cell carcinoma and post-neuroblastoma renal cell carcinoma.

In some embodiments, the prostate cancer is acinar adenocarcinoma, ductal adenocarcinoma, transitional cell (or urothelial) cancer, squamous cell cancer, carcinoid, small cell cancer, sarcomas or sarcomatoid cancers.

In some embodiments, the skin cancer is basal cell carcinoma, squamous cell carcinoma or malignant melanoma. In some embodiments, the basal cell carcinoma is further classified as nodular and nodular-ulcerative basal cell carcinoma, pigmented basal cell carcinoma, superficial basal cell carcinoma and morphoeic basal cell carcinoma. In some embodiments, the squamous cell carcinoma is further classified as Bowen's disease, papillary thyroid carcinoma, verrucous squamous cell carcinoma, papillary squamous cell carcinoma, squamous cell carcinoma, large cell keratinizing squamous cell carcinoma, large cell nonkeratinizing squamous cell carcinoma, small cell keratinizing squamous cell carcinoma, spindle cell squamous cell carcinoma, adenoid/pseudoglandular squamous cell carcinoma, intraepidermal squamous cell carcinoma, lymphoepithelial carcinoma, keratoacanthoma, Erythroplasia of Queyrat and Marjolin's ulcer. In some embodiments, the malignant melanoma is further classified as lentigo maligna, lentigo maligna melanoma, superficial spreading melanoma, acral lentiginous melanoma, mucosal melanoma, nodular melanoma, polypoid melanoma, desmoplastic melanoma, amelanotic melanoma, soft-tissue melanoma, melanoma with small nevus-like cells, melanoma with features of a spitz nevus and uveal melanoma.

In some embodiments, the cancer is a hematologic cancer. In some embodiments, the hematologic cancer is a leukemia, a lymphoma, a myeloma, a non-Hodgkin's lymphoma, a Hodgkin's lymphoma, or a B-cell malignancy. In some embodiments, the hematologic cancer is selected from acute lymphoblastic leukemia (ALL), acute myelogenous leukemia (AML), chronic lymphocytic leukemia (CLL), chronic myelogenous leukemia (CML), hairy cell leukemia (HCL), T-cell prolymphocytic leukemia (T-PLL), large granular lymphocytic leukemia, adult T-cell leukemia, aggressive NK cell leukemia, small lymphocytic lymphoma (SLL), high risk CLL, non-CLL/SLL lymphoma, follicular lymphoma (FL), diffuse large B-cell lymphoma (DLBCL), activated B-cell diffuse large B-cell lymphoma (ABC-DLBCL), germinal center diffuse large B-cell lymphoma (GCB DLBCL), double-hit diffuse large B-cell lymphoma (DH-DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, lymphomatoid granulomatosis, peripheral T-cell lymphoma not otherwise specified (PTCL-NOS), anaplastic large cell lymphoma, angioimmunoblastic lymphoma, cutaneous T-cell lymphoma, adult T-cell leukemia/lymphoma (ATLL), blastic NK-cell lymphoma, enteropathy-type T-cell lymphoma, hepatosplenic gamma-delta T-cell lymphoma, lymphoblastic lymphoma, nasal NK/T-cell lymphomas, Sezary syndrome, AIDS-related lymphoma, treatment-related T-cell lymphomas, classical non-Hodgkin lymphoma (e.g. nodular sclerosing, mixed cellularity, lymphocyte rich and lymphocyte depleted) and nodular lymphocyte predominant type.

### Fibrosis

In some embodiments, described herein is a method of treating fibrosis in a mammal comprising inhibiting the activity of one or more deubiquitinase enzymes in the mammal. In some embodiments, the method comprises administering a compound described herein, or a pharmaceutically acceptable salt thereof, to the mammal with a fibrosis. In some embodiments, disclosed herein are methods of treating fibrosis with a compound disclosed herein.

“Fibrosis,” as used herein, refers to the accumulation of extracellular matrix constituents that occurs following trauma, inflammation, tissue repair, immunological reactions, cellular hyperplasia, and neoplasia.

In some embodiments, disclosed herein is a method of reducing fibrosis in a tissue comprising contacting a fibrotic cell or tissue with a compound disclosed herein, in an amount sufficient to decrease or inhibit the fibrosis. In some embodiments, the fibrosis includes a fibrotic condition.

In some embodiments, reducing fibrosis, or treatment of a fibrotic condition, includes reducing or inhibiting one or more of: formation or deposition of extracellular matrix proteins; the number of pro-fibrotic cell types (e.g., fibroblast or immune cell numbers); cellular collagen or hydroxyproline content within a fibrotic lesion; expression or activity of a fibrogenic protein; or reducing fibrosis associated with an inflammatory response.

In some embodiments, the fibrotic condition is primary fibrosis. In some embodiments, the fibrotic condition is idiopathic. In some embodiments, the fibrotic condition is associated with (e.g., is secondary to) a disease; a toxin; an insult (e.g., an environmental hazard); a medical treatment, or a combination thereof.

In some embodiments, the fibrotic condition is a fibrotic condition of the lung (pulmonary fibrosis), a fibrotic condition of the liver (hepatic fibrosis), a fibrotic condition of the heart or vasculature (cardiac fibrosis), a fibrotic condition of the kidney (renal fibrosis), a fibrotic condition of the skin, a fibrotic condition of the gastrointestinal tract, or a combination thereof.

In some embodiments, the fibrotic condition is a fibrotic condition of the lung. In some embodiments, the fibrotic condition of the lung is chosen from one or more of: pulmonary fibrosis, idiopathic pulmonary fibrosis (IPF), usual interstitial pneumonitis (UIP), interstitial lung disease, cryptogenic fibrosing alveolitis (CFA), bronchiolitis obliterans, or bronchiectasis. In some embodiments, the fibrotic condition of the lung treated with the methods of the invention is associated with (e.g., secondary to) a cancer treatment.

In some embodiments, the fibrotic condition is a fibrotic condition of the liver.

In some embodiments, the fibrotic condition is a fibrotic condition of the lung.

In some embodiments, the fibrotic condition is a fibrotic condition of the heart.

In some embodiments, the fibrotic condition is a fibrotic condition of the kidney.

In some embodiments, the fibrotic condition is a fibrotic condition of the skin.

In some embodiments, the fibrotic condition is a fibrotic condition of the gastrointestinal tract.

In some embodiments, the fibrotic condition is a fibrotic condition of the peritoneum.

In some embodiments, the fibrotic condition is a fibrotic condition of the bone marrow.

In some embodiments, the fibrotic condition is a fibrotic condition of the eye.

### Additional Diseases

In some embodiments, a deubiquitinase inhibitor is used in the treatment of an autoimmune disease or condition. In some embodiments, the autoimmune disease or condition is lupus, rheumatoid arthritis, psoriatic arthritis, osteoarthritis, Still's disease, juvenile arthritis, diabetes, myasthenia gravis, Hashimoto's thyroiditis, Ord's thyroiditis, Graves' disease, Sjögren's syndrome, multiple sclerosis, Guillain-Barré syndrome, acute disseminated encephalomyelitis, Addison's disease, opsoclonus-myoclonus syndrome, ankylosing spondylitisis, antiphospholipid antibody syndrome, aplastic anemia, autoimmune hepatitis, coeliac disease, Goodpasture's syndrome, Kawasaki's Disease, idiopathic thrombocytopenic purpura, multiple sclerosis, optic neuritis, uveitis, scleroderma, primary biliary cirrhosis, Reiter's syndrome, Takayasu's arteritis, temporal arteritis, optic neuritis, warm autoimmune hemolytic anemia, Wegener's granulomatosis, psoriasis, diabetes mellitus (Type I), alopecia universalis, Behçet's disease, chronic fatigue syndrome, coeliac disease, Crohn's disease, ulcerative colitis, dysautonomia, endometriosis, interstitial cystitis, neuromyotonia, scleroderma, or vulvodynia.

In some embodiments, a deubiquitinase inhibitor is used in the treatment of an inflammatory disease or condition. In some embodiments, the inflammatory disease or condition is asthma, appendicitis, blepharitis, bronchiolitis, bronchitis, bursitis, cervicitis, cholangitis, cholecystitis, colitis, conjunctivitis, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, hepatitis, hidradenitis suppurativa, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, proctitis, prostatitis, pyelonephritis, rhinitis, salpingitis, sinusitis, stomatitis, synovitis, tendonitis, tonsillitis, uveitis, vaginitis, vasculitis, or vulvitis.

In some embodiments, described herein is a method of treating a neurodegenerative disease or condition in a mammal comprising inhibiting the activity of one or more deubiquitinase enzymes in the mammal. In some embodiments, the method comprises administering a compound described herein, or a pharmaceutically acceptable salt thereof, to the mammal with a neurodegenerative disease.

In some embodiments, the neurodegenerative disease is selected from Alzheimer's disease, Parkinson's disease, Huntington disease, amyotrophic lateral sclerosis (ALS), spinocerebellar ataxias and Charcot-Marie-Tooth disease.

In some embodiments, described herein is a method of treating an infection in a mammal comprising inhibiting the activity of one or more deubiquitinase enzymes in the mammal. In some embodiments, the method comprises administering a compound described herein, or a pharmaceutically acceptable salt thereof, to the mammal with an infection.

In some embodiments, the infection is caused by a bacterium, a virus, a fungus or a parasite. In some embodiments, the infection is caused by a virus. Examplary virus include, but are not limited to, cytomegalovirus, encephalomyocarditis virus (EMCV), Epstein-Barr virus, hepatitis B, hepatitis C, Kaposi's sarcoma herpesvirus, human immunodeficiency virus, human papillomavirus, human T-cell leukemia virus-1influenza virus, Merkel cell polyomavirus and respiratory syncytial virus.

In some embodiments, the infection is caused by a bacterium. Examplary bacteria include, but are not limited to, Burkholderia cenocepacia, Chlamydia pneumoniae, Escherichia coli, Helicobacter pylori, Listeria monocytogenes, Mycobacterium tuberculosis, Pseudomonas aeruginosa, Salmonella typhi, Salmonella enteric, Shigella flexneri, Stenotrophomonas maltopilia, Sthaphylococcus aureus and methicillin-resistant Staphylococcus Aureus (MRSA).

In some embodiments, the infection is caused by a fungus. Examplary fungi include, but are not limited to, Aspergillus fumigates, Aspergillus terreus, Peniciliun sp. Chrysonilia sp, Candida albicans, Candida glabrata and Exophiala dermatitidis.

In some embodiments, the infection is caused by a parasite. Examplary parasites include, but are not limited to, Clonorchis sinensis, liver flukes, Opisthorchis viverrini, Schistosoma haematobium and Schistosoma japonicum.

### Compounds

Compounds described herein, including pharmaceutically acceptable salts, prodrugs, active metabolites and pharmaceutically acceptable solvates thereof, are DUB inhibitors.

In one aspect, the DUB inhibitor contemplated in any of the pharmaceutical compositions, methods and uses described herein is any small molecule compound that inhibits the activity of at least one deubiquitinase enzyme. In some embodiments, the at least one deubiquitinase enzyme is USP9x, USP2, USP5, USP14, USP24 and/or UCHL5.

In some embodiments, the DUB inhibitor is a small molecule compound. In some embodiments, the DUB inhibitor is a cyano-substituted acrylamide small molecule compound. In some embodiments, the DUB inhibitor is a caffeic acid amide derivative. In some embodiments, the DUB inhibitor is a small molecule compound that has the following structure:

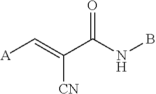

wherein,
A is a substituted or unsubstituted heteroaryl or substituted or unsubstituted aryl; and
B is substituted or unsubstituted -alkylene-aryl or substituted or unsubstituted -alkylene-heteroaryl.

In some embodiments, A is a substituted or unsubstituted aryl. In some embodiments, A is a substituted or unsubstituted phenyl. In some embodiments, A is a substituted or unsubstituted naphthyl.

In some embodiments, A is a substituted or unsubstituted heteroaryl. In some embodiments, A is a substituted or unsubstituted monocyclic heteroaryl. Monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. In some embodiments, A is a substituted or unsubstituted 5-membered monocyclic heteroaryl. In some embodiments, A is a substituted or unsubstituted 5-membered monocyclic heteroaryl selected from among imidazolyl, pyrazolyl, triazolyl, tetrazolyl, furyl, thienyl, isoxazolyl, thiazolyl, oxazolyl, isothiazolyl, pyrrolyl, oxadiazolyl, thiadiazolyl, and furazanyl. In some embodiments, A is a substituted or unsubstituted 6-membered monocyclic heteroaryl. In some embodiments, A is a substituted or unsubstituted 6-membered monocyclic heteroaryl selected from among pyridinyl, pyrimidinyl, pyrazinyl, pyridazinyl, and triazinyl. In some embodiments, A is a substituted or unsubstituted pyridinyl.

In some embodiments B is a substituted or unsubstituted -methylene-phenyl (i.e. a substituted or unsubstituted benzyl).

In one aspect, described herein is a DUB inhibitor that has the structure of Formula (I), or a pharmaceutically acceptable salt, or solvate thereof:

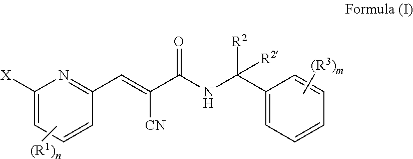

wherein,
X is H, F, Cl, Br, alkyl, —O-alkyl, —S-alkyl, —NH₂ or —NH-alkyl;
R¹ is H, halogen, —OH, CN, NO₂, unsubstituted or substituted alkyl, unsubstituted or substituted cycloalkyl, —O-(unsubstituted or substituted alkyl), —S-(unsubstituted or substituted alkyl), or —NH-(unsubstituted or substituted alkyl);
or R¹ and X are taken together with the intervening atoms connecting R¹ and X to form a heterocyclic ring with 1 or 2 heteroatoms selected from O, N, and S;
R² is H, unsubstituted or substituted alkyl, unsubstituted or substituted heteroalkyl, unsubstituted or substituted cycloalkyl, unsubstituted or substituted heterocycloalkyl, or unsubstituted or substituted benzyl;
or R² is -L¹-R⁶ where,
   L¹ is bond, unsubstituted or substituted alkyl, unsubstituted or substituted cycloalkyl, or unsubstituted or substituted heteroalkyl;
   R⁶ is unsubstituted or substituted cycloalkyl, unsubstituted or substituted heterocycloalkyl, unsubstituted or substituted aryl, or unsubstituted or substituted heteroaryl;
R^{2′} is H or unsubstituted or substituted alkyl;
each R³ is independently selected from H, halogen, —CN, —NO₂, —OH, —OR⁴, —SR⁴, —S(═O)R⁴, —S(═O)₂R⁴, —S(═O)₂N(R⁵)₂, —NR⁵S(═O)₂R⁴, —C(═O)R⁴, —OC(═O)R⁴, —CO₂R⁵, —OCO₂R⁴, —N(R⁵)₂, —C(═O)N(R⁵)₂, —OC(═O)N(R⁵)₂, —NHC(═O)R⁴, —NHC(═O)OR⁴, unsubstituted or substituted alkyl, unsubstituted or substituted alkenyl, unsubstituted or substituted alkynyl, unsubstituted or substituted heteroalkyl, unsubstituted or substituted cycloalkyl, unsubstituted or substituted aryl, unsubstituted or substituted heteroaryl, and -L²-L³-L⁴-L⁵-R⁷;
   L² is absent, —O—, —S—, —S(O)—, —S(O)₂₋, —NR⁴—, —CH(OH)—, —C(═O)—, —C(═O)NH—, —NHC(═O)—, —C(═O)O—, —OC(═O)—, —CH(═N)—, —CH(═N—NH)—, —CCH₃(═N)—, —CCH₃(═N—NH)—, —OC(═O)NH—, —NHC(═O)NH—, —NHC(═O)O—, —(CH₂)ₚ—, or —(OCH₂CH₂)ₚ—, p is 1, 2, 3, or 4;
   L³ is absent, unsubstituted or substituted alkylene, unsubstituted or substituted heteroalkylene, unsubstituted or substituted alkenylene, unsubstituted or substituted alkynylene, unsubstituted or substituted cycloalkylene, unsubstituted or substituted heterocycloalkylene, unsubstituted or substituted arylene, unsubstituted or substituted heteroarylene, or —(OCH₂CH₂)ₚ—, p is 1, 2, 3, or 4;
   L⁴ is absent, —O—, —S—, —S(O)—, —S(O)₂₋, —NR⁴—, —CH(OH)—, —C(═O)—, —C(═O)NH—, —NHC(═O)—, —C(═O)O—, —OC(═O)—, —OC(═O)NH—, —NHC(═O)NH—, or —NHC(═O)O—;
   L⁵ is absent, unsubstituted or substituted alkylene, unsubstituted or substituted heteroalkylene;
   R⁷ is H, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, or unsubstituted or substituted heteroaryl;
R⁴ is C₁-C₆alkyl, C₁-C₆fluoroalkyl, C₁-C₆deuteroalkyl, C₃-C₆cycloalkyl, a substituted or unsubstituted phenyl, a substituted or unsubstituted monocyclic heteroaryl, or a substituted or unsubstituted bicyclic heteroaryl;
each R⁵ is independently selected from H, C₁-C₆alkyl, C₁-C₆fluoroalkyl, C₁-C₆deuteroalkyl, C₃-C₆cycloalkyl, a substituted or unsubstituted phenyl, or a substituted or unsubstituted monocyclic heteroaryl; or
two R⁵ groups attached to the same N atom are taken together with the N atom to which they are attached to form a substituted or unsubstituted heterocycle;
m is 0, 1, 2, 3, or 4;
n is 0, 1, 2 or 3.

For any and all embodiments, substitutents are selected from among a subset of the listed alternatives. For example, in some embodiments, X is F, Cl, Br, —OH, alkyl, —O-alkyl, —S-alkyl, —NH₂ or —NH-alkyl. In other embodiments, X is F, Cl, or Br. In yet other embodiments, X is Br.

In some embodiments, R¹ is H or halogen. In some embodiments, R¹ is H, F, Cl or Br.

In some embodiments, R² is H or unsubstituted alkyl. In some embodiments, R² is H, methyl, ethyl, propyl, or butyl. In some embodiments, R² is H, methyl, ethyl, n-propyl, iso-propyl, n-butyl, sec-butyl, iso-butyl or tert-butyl.

In some embodiments, R^{2′} is H, or unsubstituted alkyl. In some embodiments, R^{2′} is H, methyl, ethyl, propyl, or butyl.

In some embodiments, R² is H or unsubstituted alkyl; and R^{2′} is H.

In some embodiments, R² is H, methyl, ethyl, propyl, or butyl; and R^{2′} is H. In some embodiments, R² is H, methyl, ethyl, n-propyl, iso-propyl, n-butyl, sec-butyl, iso-butyl or tert-butyl; and R^{2′} is H. In some embodiments, R² is methyl, ethyl, n-propyl, iso-propyl, n-butyl, sec-butyl, iso-butyl or tert-butyl; and R^{2′} is H. In some embodiments, R² is n-propyl; and R^{2′} is H.

In some embodiments, each R³ is independently selected from H, halogen, —CN, —OH, —OR⁴, or unsubstituted or substituted alkyl. In some embodiments, each R³ is H.

In some embodiments, each R³ is -L²-L³-L⁴-L⁵-R⁷.

In some embodiments, L² is absent, —O—, —NR⁴—, —C(═O)—, —C(═O)NH—, —NHC(═O)—, —C(═O)O—, —OC(═O)—, —OC(═O)NH—, —NHC(═O)NH—, —NHC(═O)O—, —(CH₂)ₚ—, or —(OCH₂CH₂)ₚ—. In some embodiments, L² is absent, —O—, —NR⁴—, —C(═O)—, —C(═O)NH—, —NHC(═O)—, —C(═O)O—, —OC(═O)—, —(CH₂)ₚ—, or —(OCH₂CH₂)ₚ—. In some embodiments, L² is absent, —O—, or —(CH₂)ₚ—. In some embodiments, L² is absent or —(CH₂)ₚ—.

In some embodiments, L³ is absent, or unsubstituted or substituted alkylene. In some embodiments, L³ is absent.

In some embodiments, L⁴ is absent, —O—, —NR⁴—, —C(═O)—, —C(═O)NH—, —NHC(═O)—, —C(═O)O—, or —OC(═O)—. In some embodiments, L⁴ is absent, —O—, —C(═O)NH—, —NHC(═O)—, —C(═O)O—, or —OC(═O)—. In some embodiments, L⁴ is absent, —C(═O)O—, or —OC(═O)—.

In some embodiments, L⁵ is absent, or unsubstituted or substituted alkylene. In some embodiments, L⁵ is unsubstituted or substituted alkylene.

In some embodiments, -L²-L³-L⁴-L⁵- is

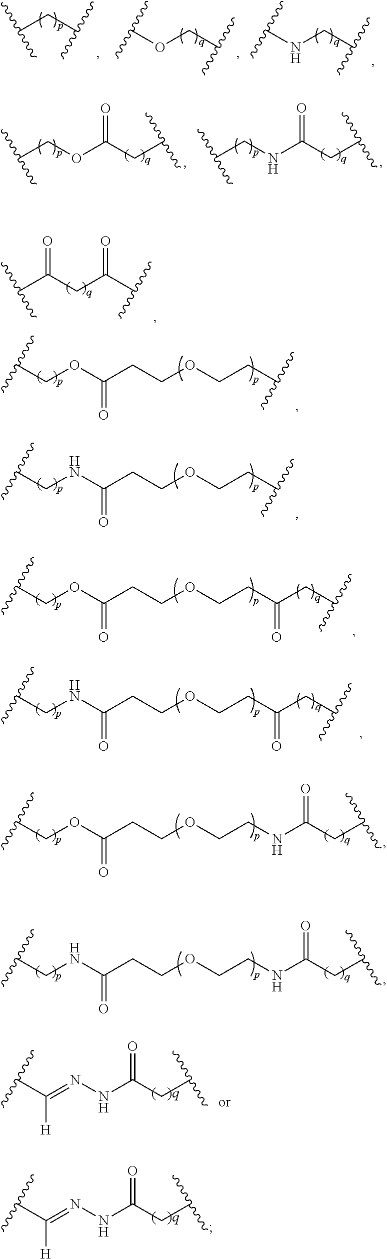

q is 0, 1, 2, 3, 4 or 5; and each p is independently 0, 1, 2, 3, or 4.

In some embodiments, each q is independently 1, 2, 3, or 4. In some embodiments, q is 0, 1, 2, 3, or 4. In some embodiments, each p is independently 0, 1, 2, 3, or 4. In some embodiments, each p is independently 1, 2, 3, or 4.

In some embodiments, R⁷ is unsubstituted or substituted heterocycloalkyl. In some embodiments, R⁷ is unsubstituted or substituted monocyclic heterocycloalkyl or unsubstituted or substituted bicyclic heterocycloalkyl. In some embodiments, R⁷ is unsubstituted or substituted maleimidyl, unsubstituted or substituted morpholinyl, unsubstituted or substituted pyrrolidnyl, unsubstituted or substituted piperazinyl, or unsubstituted or substituted biotinyl. In some embodiments, R⁷ is unsubstituted or substituted maleimidyl.

In some embodiments, the deubiquitinase inhibitor is a compound in the following table:

TABLE 1

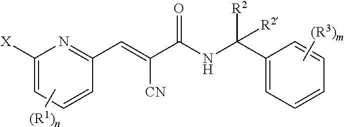

| X | R¹ | R² | R^{2′} | R³ |
|---|---|---|---|---|
| Br | H | Me | H | H |
| Br | H | Et | H | H |
| Br | H | Pr | H | H |
| Br | H | iPr | H | H |
| F | H | Me | H | H |
| F | H | Et | H | H |
| F | H | Pr | H | H |
| Cl | H | Me | H | H |
| Cl | H | Et | H | H |
| Cl | H | Pr | H | H |
| Br | 5-F | Me | H | H |
| Br | 5-F | Et | H | H |
| Br | 5-F | Pr | H | H |
| Cl | 5-F | Me | H | H |
| Cl | 5-F | Et | H | H |
| Cl | 5-F | Pr | H | H |
| F | 5-F | Me | H | H |
| F | 5-F | Et | H | H |
| F | 5-F | Pr | H | H |
| Br | 5-Cl | Me | H | H |
| Br | 5-Cl | Et | H | H |
| Br | 5-Cl | Pr | H | H |
| Cl | 5-Cl | Me | H | H |
| Cl | 5-Cl | Et | H | H |
| Cl | 5-Cl | Pr | H | H |
| F | 5-Cl | Me | H | H |
| F | 5-Cl | Et | H | H |
| F | 5-Cl | Pr | H | H |
| Br | 4-F | Me | H | H |
| Br | 4-F | Et | H | H |
| Br | 4-F | Pr | H | H |
| Cl | 4-F | Me | H | H |
| Cl | 4-F | Et | H | H |
| Cl | 4-F | Pr | H | H |
| F | 4-F | Me | H | H |
| F | 4-F | Et | H | H |
| F | 4-F | Pr | H | H |
| Br | 4-Cl | Me | H | H |
| Br | 4-Cl | Et | H | H |
| Br | 4-Cl | Pr | H | H |
| Cl | 4-Cl | Me | H | H |
| Cl | 4-Cl | Et | H | H |
| Cl | 4-Cl | Pr | H | H |
| F | 4-Cl | Me | H | H |
| F | 4-Cl | Et | H | H |
| F | 4-Cl | Pr | H | H |
| Br | 3-F | Me | H | H |
| Br | 3-F | Et | H | H |
| Br | 3-F | Pr | H | H |
| Cl | 3-F | Me | H | H |
| Cl | 3-F | Et | H | H |
| Cl | 3-F | Pr | H | H |
| F | 3-F | Me | H | H |
| F | 3-F | Et | H | H |
| F | 3-F | Pr | H | H |
| Br | 3-Cl | Me | H | H |
| Br | 3-Cl | Et | H | H |
| Br | 3-Cl | Pr | H | H |
| Cl | 3-Cl | Me | H | H |
| Cl | 3-Cl | Et | H | H |
| Cl | 3-Cl | Pr | H | H |
| F | 3-Cl | Me | H | H |
| F | 3-Cl | Et | H | H |
| F | 3-Cl | Pr | H | H |
| F | H | Pr | H | 4-OMe |
| Cl | H | Pr | H | 4-OMe |
| Br | H | Pr | H | 4-OMe |
| F | H | Pr | H | 3-OMe |
| Cl | H | Pr | H | 3-OMe |
| Br | H | Pr | H | 3-OMe |
| H | 3,5-diBr | Me | H | H |
| H | 3,5-diBr | Et | H | H |
| H | 3,5-diBr | Pr | H | H |
| H | 3-Cl, 5-F | Me | H | H |
| H | 3-Cl, 5-F | Et | H | H |
| H | 3-Cl, 5-F | Pr | H | H |
| H | 3,5-diCl | Me | H | H |
| H | 3,5-diCl | Et | H | H |
| H | 3,5-diCl | Pr | H | H |
| H | 3-Br, 5-F | Me | H | H |
| H | 3-Br, 5-F | Et | H | H |
| H | 3-Br, 5-F | Pr | H | H |
| H | 3-F, 5-Cl | Me | H | H |
| H | 3-F, 5-Cl | Et | H | H |
| H | 3-F, 5-Cl | Pr | H | H |
| H | 3-F, 5-Br | Me | H | H |
| H | 3-F, 5-Br | Et | H | H |
| H | 3-F, 5-Br | Pr | H | H |
| H | 3,5-DiF | Me | H | H |
| H | 3,5-DiF | Et | H | H |
| H | 3,5-DiF | Pr | H | H |
| H | 3-Br, 5-Cl | Me | H | H |
| H | 3-Br, 5-Cl | Et | H | H |
| H | 3-Br, 5-Cl | Pr | H | H |
| H | 3-Cl, 5-Br | Me | H | H |
| H | 3-Cl, 5-Br | Et | H | H |
| H | 3-Cl, 5-Br | Pr | H | H |

Additional DUB inhibitor compounds include, but are not limited to WP1130, WP1066, and those compounds described in U.S. Pat. No. 7,745,468; US2005/0277680; US2011/0021805; US2011/0275577; US2012/0077806 and those described in Peng Bioorg Med Chem., 2014, 22, p 1450; Peng 2011, Bioorg Med Chem., 19, p 7194.

In some embodiments, compounds described herein inhibit the activity of one or more kinases. In some embodiments, the one or more kinases include non-receptor tyrosine kinases. In some embodiments, the one or more kinases include one or more Janus kinases. In some embodiments, the one or more kinases include Jak/Stat kinases. In some embodiments, the one or more kinases include Jak/Stat, and/or Jak2/Stat3. In some embodiments, compounds described herein are used to treat any one of the diseases or conditions described herein by inhibiting the activity of one or more kinases. In some embodiments, compounds described herein are used to treat cancer in a mammal by inhibiting the activity of one or more kinases.

In one aspect, compounds described herein are in the form of pharmaceutically acceptable salts. As well, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

“Pharmaceutically acceptable,” as used herein, refers a material, such as a carrier or diluent, which does not abrogate the biological activity or properties of the compound, and is relatively nontoxic, i.e., the material is administered to an individual without causing undesirable biological effects or interacting in a deleterious manner with any of the components of the composition in which it is contained.

The term “pharmaceutically acceptable salt” refers to a form of a therapeutically active agent that consists of a cationic form of the therapeutically active agent in combination with a suitable anion, or in alternative embodiments, an anionic form of the therapeutically active agent in combination with a suitable cation. Handbook of Pharmaceutical Salts: Properties, Selection and Use. International Union of Pure and Applied Chemistry, Wiley-VCH 2002. S. M. Berge, L. D. Bighley, D. C. Monkhouse, J. Pharm. Sci. 1977, 66, 1-19. P. H. Stahl and C. G. Wermuth, editors, Handbook of Pharmaceutical Salts: Properties, Selection and Use, Weinheim/Zürich:Wiley-VCH/VHCA, 2002. Pharmaceutical salts typically are more soluble and more rapidly soluble in stomach and intestinal juices than non-ionic species and so are useful in solid dosage forms. Furthermore, because their solubility often is a function of pH, selective dissolution in one or another part of the digestive tract is possible and this capability can be manipulated as one aspect of delayed and sustained release behaviours. Also, because the salt-forming molecule can be in equilibrium with a neutral form, passage through biological membranes can be adjusted.

In some embodiments, pharmaceutically acceptable salts are obtained by reacting a compound described herein with an acid. In some embodiments, the compound described herein (i.e. free base form) is basic and is reacted with an organic acid or an inorganic acid. Inorganic acids include, but are not limited to, hydrochloric acid, hydrobromic acid, sulfuric acid, phosphoric acid, nitric acid, and metaphosphoric acid. Organic acids include, but are not limited to, 1-hydroxy-2-naphthoic acid; 2,2-dichloroacetic acid; 2-hydroxyethanesulfonic acid; 2-oxoglutaric acid; 4-acetamidobenzoic acid; 4-aminosalicylic acid; acetic acid; adipic acid; ascorbic acid (L); aspartic acid (L); benzenesulfonic acid; benzoic acid; camphoric acid (+); camphor-10-sulfonic acid (+); capric acid (decanoic acid); caproic acid (hexanoic acid); caprylic acid (octanoic acid); carbonic acid; cinnamic acid; citric acid; cyclamic acid; dodecylsulfuric acid; ethane-1,2-disulfonic acid; ethanesulfonic acid; formic acid; fumaric acid; galactaric acid; gentisic acid; glucoheptonic acid (D); gluconic acid (D); glucuronic acid (D); glutamic acid; glutaric acid; glycerophosphoric acid; glycolic acid; hippuric acid; isobutyric acid; lactic acid (DL); lactobionic acid; lauric acid; maleic acid; malic acid (−L); malonic acid; mandelic acid (DL); methanesulfonic acid; naphthalene-1,5-disulfonic acid; naphthalene-2-sulfonic acid; nicotinic acid; oleic acid; oxalic acid; palmitic acid; pamoic acid; phosphoric acid; proprionic acid; pyroglutamic acid (−L); salicylic acid; sebacic acid; stearic acid; succinic acid; sulfuric acid; tartaric acid (+L); thiocyanic acid; toluenesulfonic acid (p); and undecylenic acid.

In some embodiments, a compound described herein is prepared as a chloride salt, sulfate salt, bromide salt, mesylate salt, maleate salt, citrate salt or phosphate salt. In some embodiments, a compound described herein is prepared as a hydrochloride salt.

In some embodiments, pharmaceutically acceptable salts are obtained by reacting a compound described herein with a base. In some embodiments, the compound described herein is acidic and is reacted with a base. In such situations, an acidic proton of the compound described herein is replaced by a metal ion, e.g., lithium, sodium, potassium, magnesium, calcium, or an aluminum ion. In some cases, compounds described herein coordinate with an organic base, such as, but not limited to, ethanolamine, diethanolamine, triethanolamine, tromethamine, meglumine, N-methylglucamine, dicyclohexylamine, tris(hydroxymethyl)methylamine. In other cases, compounds described herein form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, potassium carbonate, sodium hydroxide, lithium hydroxide, and the like. In some embodiments, the compounds provided herein are prepared as a sodium salt, calcium salt, potassium salt, magnesium salt, meglumine salt, N-methylglucamine salt or ammonium salt. In some embodiments, the compounds provided herein are prepared as a sodium salt.

It should be understood that a reference to a pharmaceutically acceptable salt includes the solvent addition forms. In some embodiments, solvates contain either stoichiometric or non-stoichiometric amounts of a solvent, and are formed during the process of crystallization with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. In addition, the compounds provided herein optionally exist in unsolvated as well as solvated forms.

The methods and formulations described herein include the use of N-oxides (if appropriate), crystalline forms (also known as polymorphs), or pharmaceutically acceptable salts of compounds described herein, as well as active metabolites of these compounds having the same type of activity.

In some embodiments, sites on the organic radicals (e.g. alkyl groups, aromatic rings) of compounds described herein are susceptible to various metabolic reactions. Incorporation of appropriate substituents on the organic radicals will reduce, minimize or eliminate this metabolic pathway. In specific embodiments, the appropriate substituent to decrease or eliminate the susceptibility of the aromatic ring to metabolic reactions is, by way of example only, a halogen, deuterium, an alkyl group, a haloalkyl group, or a deuteroalkyl group.

In another embodiment, the compounds described herein are labeled isotopically (e.g. with a radioisotope) or by another other means, including, but not limited to, the use of chromophores or fluorescent moieties, bioluminescent labels, or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, fluorine and chlorine, such as, for example, ²H, ³H, ¹³C, ¹⁴C, ¹⁵N, ¹⁸O, ¹⁷O, ³⁵S, ¹⁸F, ³⁶Cl. In one aspect, isotopically-labeled compounds described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements.

In some embodiments, the compounds described herein possess one or more stereocenters and each stereocenter exists independently in either the R or S configuration. The compounds presented herein include all diastereomeric, enantiomeric, atropisomers, and epimeric forms as well as the appropriate mixtures thereof. The compounds and methods provided herein include all cis, trans, syn, anti, entgegen (E), and zusammen (Z) isomers as well as the appropriate mixtures thereof.

Individual stereoisomers are obtained, if desired, by methods such as, stereoselective synthesis and/or the separation of stereoisomers by chiral chromatographic columns. In certain embodiments, compounds described herein are prepared as their individual stereoisomers by reacting a racemic mixture of the compound with an optically active resolving agent to form a pair of diastereoisomeric compounds/salts, separating the diastereomers and recovering the optically pure enantiomers. In some embodiments, resolution of enantiomers is carried out using covalent diastereomeric derivatives of the compounds described herein. In another embodiment, diastereomers are separated by separation/resolution techniques based upon differences in solubility. In other embodiments, separation of steroisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley and Sons, Inc., 1981. In some embodiments, stereoisomers are obtained by stereoselective synthesis.

In some embodiments, compounds described herein are prepared as prodrugs. A “prodrug” refers to an agent that is converted into the parent drug in vivo. Prodrugs are often useful because, in some situations, they are easier to administer than the parent drug. They are, for instance, bioavailable by oral administration whereas the parent is not. Further or alternatively, the prodrug also has improved solubility in pharmaceutical compositions over the parent drug. In some embodiments, the design of a prodrug increases the effective water solubility. An example, without limitation, of a prodrug is a compound described herein, which is administered as an ester (the “prodrug”) but then is metabolically hydrolyzed to provide the active entity. A further example of a prodrug is a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the compound. In certain embodiments, a prodrug is enzymatically metabolized by one or more steps or processes to the biologically, pharmaceutically or therapeutically active form of the compound.

Prodrugs of the compounds described herein include, but are not limited to, esters, ethers, carbonates, thiocarbonates, N-acyl derivatives, N-acyloxyalkyl derivatives, quaternary derivatives of tertiary amines, N-Mannich bases, Schiff bases, amino acid conjugates, phosphate esters, and sulfonate esters. See for example Design of Prodrugs, Bundgaard, A. Ed., Elseview, 1985 and Method in Enzymology, Widder, K. et al., Ed.; Academic, 1985, vol. 42, p. 309-396; Bundgaard, H. “Design and Application of Prodrugs” in A Textbook of Drug Design and Development, Krosgaard-Larsen and H. Bundgaard, Ed., 1991, Chapter 5, p. 113-191; and Bundgaard, H., Advanced Drug Delivery Review, 1992, 8, 1-38, each of which is incorporated herein by reference. In some embodiments, a hydroxyl group in the compounds disclosed herein is used to form a prodrug, wherein the hydroxyl group is incorporated into an acyloxyalkyl ester, alkoxycarbonyloxyalkyl ester, alkyl ester, aryl ester, phosphate ester, sugar ester, ether, and the like. In some embodiments, a hydroxyl group in the compounds disclosed herein is a prodrug wherein the hydroxyl is then metabolized in vivo to provide a carboxylic acid group. In some embodiments, a carboxyl group is used to provide an ester or amide (i.e. the prodrug), which is then metabolized in vivo to provide a carboxylic acid group. In some embodiments, compounds described herein are prepared as alkyl ester prodrugs.

Prodrug forms of the herein described compounds, wherein the prodrug is metabolized in vivo to produce a compound described herein as set forth herein are included within the scope of the claims. In some cases, some of the herein-described compounds are a prodrug for another derivative or active compound.

In additional or further embodiments, the compounds described herein are metabolized upon administration to an organism in need to produce a metabolite that is then used to produce a desired effect, including a desired therapeutic effect.

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to a compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulphydryl groups. Metabolites of the compounds disclosed herein are optionally identified either by administration of compounds to a host and analysis of tissue samples from the host, or by incubation of compounds with hepatic cells in vitro and analysis of the resulting compounds.

### Synthesis of Compounds

Compounds described herein are synthesized using standard synthetic techniques or using methods known in the art in combination with methods described herein. In some embodiments, compounds described herein are prepared using the procedures described in U.S. Pat. No. 7,745,468; US2005/0277680; US2011/0021805; US2011/0275577; US2012/0077806 and those described in Peng Bioorg Med Chem., 2014, 22, p 1450; Peng 2011, Bioorg Med Chem., 19, p 7194.

Compounds are prepared using standard organic chemistry techniques such as those described in, for example, March's Advanced Organic Chemistry, 6^{th} Edition, John Wiley and Sons, Inc. Alternative reaction conditions for the synthetic transformations described herein may be employed such as variation of solvent, reaction temperature, reaction time, as well as different chemical reagents and other reaction conditions. The starting materials are available from commercial sources or are readily prepared.

### CERTAIN TERMINOLOGY

Unless otherwise stated, the following terms used in this application have the definitions given below. The use of the term “including” as well as other forms, such as “include”, “includes,” and “included,” is not limiting. The section headings used herein are for organizational purposes only and are not to be construed as limiting the subject matter described.

As used herein, C₁-Cₓ includes C₁-C₂, C₁-C₃ . . . C₁-Cₓ. By way of example only, a group designated as “C₁-C₄” indicates that there are one to four carbon atoms in the moiety, i.e. groups containing 1 carbon atom, 2 carbon atoms, 3 carbon atoms or 4 carbon atoms. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl.

An “alkyl” group refers to an aliphatic hydrocarbon group. The alkyl group is branched or straight chain. In some embodiments, the “alkyl” group has 1 to 10 carbon atoms, i.e. a C₁-C₁₀alkyl. Whenever it appears herein, a numerical range such as “1 to 10” refers to each integer in the given range; e.g., “1 to 10 carbon atoms” means that the alkyl group consist of 1 carbon atom, 2 carbon atoms, 3 carbon atoms, etc., up to and including 10 carbon atoms, although the present definition also covers the occurrence of the term “alkyl” where no numerical range is designated. In some embodiments, an alkyl is a C₁-C₆alkyl. In one aspect the alkyl is methyl, ethyl, propyl, iso-propyl, n-butyl, iso-butyl, sec-butyl, or t-butyl. Typical alkyl groups include, but are in no way limited to, methyl, ethyl, propyl, isopropyl, butyl, isobutyl, sec-butyl, tertiary butyl, pentyl, neopentyl, or hexyl.

An “alkylene” group refers to a divalent alkyl radical. Any of the above mentioned monovalent alkyl groups may be an alkylene by abstraction of a second hydrogen atom from the alkyl. In some embodiments, an alkelene is a C₁-C₆alkylene. In other embodiments, an alkylene is a C₁-C₄alkylene. Typical alkylene groups include, but are not limited to, —CH₂—, —CH(CH₃)—, —C(CH₃)₂—, —CH₂CH₂—, —CH₂CH(CH₃)—, —CH₂C(CH₃)₂—, —CH₂CH₂CH₂—, —CH₂CH₂CH₂CH₂—, —CH₂CH₂CH₂CH₂CH₂—, —CH₂CH₂CH₂CH₂CH₂CH₂—, and the like.

“Deuteroalkyl” refers to an alkyl group where 1 or more hydrogen atoms of an alkyl are replaced with deuterium.

The term “alkenyl” refers to a type of alkyl group in which at least one carbon-carbon double bond is present. In one embodiment, an alkenyl group has the formula —C(R)═CR₂, wherein R refers to the remaining portions of the alkenyl group, which may be the same or different. In some embodiments, R is H or an alkyl. Non-limiting examples of an alkenyl group include —CH═CH₂, —C(CH₃)═CH₂, —CH═CHCH₃, —C(CH₃)═CHCH₃, and —CH₂CH═CH₂.

The term “alkynyl” refers to a type of alkyl group in which at least one carbon-carbon triple bond is present. In one embodiment, an alkenyl group has the formula —C≡C—R, wherein R refers to the remaining portions of the alkynyl group. In some embodiments, R is H or an alkyl. Non-limiting examples of an alkynyl group include —C≡CH, —C≡CCH₃—C≡CCH₂CH₃, —CH₂C≡CH.

An “alkoxy” group refers to a (alkyl)O— group, where alkyl is as defined herein.

The term “alkylamine” refers to the —N(alkyl)ₓH_{y} group, where x is 0 and y is 2, or where x is 1 and y is 1, or where x is 2 and y is 0.

The term “aromatic” refers to a planar ring having a delocalized π-electron system containing 4n+2π electrons, where n is an integer. The term “aromatic” includes both carbocyclic aryl (“aryl”, e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon atoms) groups.

The term “carbocyclic” or “carbocycle” refers to a ring or ring system where the atoms forming the backbone of the ring are all carbon atoms. The term thus distinguishes carbocyclic from “heterocyclic” rings or “heterocycles” in which the ring backbone contains at least one atom which is different from carbon. In some embodiments, at least one of the two rings of a bicyclic carbocycle is aromatic. In some embodiments, both rings of a bicyclic carbocycle are aromatic.

“Bicyclic” refers to two fused rings. Fusion of the rings can occur in three ways: across a bond between two atoms, across a sequence of atoms (bridgehead), or at a single atom (spirocyclic, forming a spiro compound).

As used herein, the term “aryl” refers to an aromatic ring wherein each of the atoms forming the ring is a carbon atom. In one aspect, aryl is phenyl or a naphthyl. In some embodiments, an aryl is a phenyl. In some embodiments, an aryl is a C₆-C₁₀aryl. Depending on the structure, an aryl group is a monoradical or a diradical (i.e., an arylene group).

The term “cycloalkyl” refers to a monocyclic or polycyclic aliphatic, non-aromatic radical, wherein each of the atoms forming the ring (i.e. skeletal atoms) is a carbon atom. In some embodiments, cycloalkyls are spirocyclic or bridged compounds. In some embodiments, cycloalkyls are optionally fused with an aromatic ring, and the point of attachment is at a carbon that is not an aromatic ring carbon atom. Cycloalkyl groups include groups having from 3 to 10 ring atoms. In some embodiments, cycloalkyl groups are selected from among cyclopropyl, cyclobutyl, cyclopentyl, cyclopentenyl, cyclohexyl, cyclohexenyl, cycloheptyl, cyclooctyl, spiro[2.2]pentyl, norbornyl and bicycle[1.1.1]pentyl. In some embodiments, a cycloalkyl is a C₃-C₆cycloalkyl.

The term “halo” or, alternatively, “halogen” or “halide” means fluoro, chloro, bromo or iodo. In some embodiments, halo is fluoro, chloro, or bromo.

The term “fluoroalkyl” refers to an alkyl in which one or more hydrogen atoms are replaced by a fluorine atom. In one aspect, a fluoralkyl is a C₁-C₆fluoroalkyl.

The term “heteroalkyl” refers to an alkyl group in which one or more skeletal atoms of the alkyl are selected from an atom other than carbon, e.g., oxygen, nitrogen (e.g. —NH—, —N(alkyl)-, sulfur, or combinations thereof. A heteroalkyl is attached to the rest of the molecule at a carbon atom of the heteroalkyl. In one aspect, a heteroalkyl is a C₁-C₆heteroalkyl.

The term “heterocycle” or “heterocyclic” refers to heteroaromatic rings (also known as heteroaryls) and heterocycloalkyl rings (also known as heteroalicyclic groups) containing one to four heteroatoms in the ring(s), where each heteroatom in the ring(s) is selected from O, S and N, wherein each heterocyclic group has from 3 to 10 atoms in its ring system, and with the proviso that any ring does not contain two adjacent O or S atoms. Non-aromatic heterocyclic groups (also known as heterocycloalkyls) include rings having 3 to 10 atoms in its ring system and aromatic heterocyclic groups include rings having 5 to 10 atoms in its ring system. The heterocyclic groups include benzo-fused ring systems. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, indolin-2-onyl, isoindolin-1-onyl, isoindoline-1,3-dionyl, 3,4-dihydroisoquinolin-1(2H)-onyl, 3,4-dihydroquinolin-2(1H)-onyl, isoindoline-1,3-dithionyl, benzo[d]oxazol-2(3H)-onyl, 1H-benzo[d]imidazol-2(3H)-onyl, benzo[d]thiazol-2(3H)-onyl, and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups are either C-attached (or C-linked) or N-attached where such is possible. For instance, a group derived from pyrrole includes both pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles are optionally substituted with one or two oxo (═O) moieties, such as pyrrolidin-2-one. In some embodiments, at least one of the two rings of a bicyclic heterocycle is aromatic. In some embodiments, both rings of a bicyclic heterocycle are aromatic. Bicyclic heterocycles include two fused rings. Fusion of the rings can occur in three ways: across a bond between two atoms, across a sequence of atoms (bridgehead), or at a single atom (spirocyclic, forming a spiro compound).

The terms “heteroaryl” or, alternatively, “heteroaromatic” refers to an aryl group that includes one or more ring heteroatoms selected from nitrogen, oxygen and sulfur. Illustrative examples of heteroaryl groups include monocyclic heteroaryls and bicycicic heteroaryls. Monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, a heteroaryl contains 0-4 N atoms in the ring. In some embodiments, a heteroaryl contains 1-4 N atoms in the ring. In some embodiments, a heteroaryl contains 0-4 N atoms, 0-1 O atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉heteroaryl. In some embodiments, monocyclic heteroaryl is a C₁-C₅heteroaryl. In some embodiments, monocyclic heteroaryl is a 5-membered or 6-membered heteroaryl. In some embodiments, bicyclic heteroaryl is a C₆-C₉heteroaryl.

A “heterocycloalkyl” or “heteroalicyclic” group refers to a cycloalkyl group that includes at least one heteroatom selected from nitrogen, oxygen and sulfur. In some embodiments, a heterocycloalkyl is fused with an aryl or heteroaryl. In some embodiments, the heterocycloalkyl is oxazolidinonyl, pyrrolidinyl, tetrahydrofuranyl, tetrahydrothienyl, tetrahydropyranyl, tetrahydrothiopyranyl, piperidinyl, morpholinyl, thiomorpholinyl, piperazinyl, piperidin-2-onyl, pyrrolidine-2,5-dithionyl, pyrrolidine-2,5-dionyl, pyrrolidinonyl, imidazolidinyl, imidazolidin-2-onyl, or thiazolidin-2-onyl. The term heteroalicyclic also includes all ring forms of the carbohydrates, including but not limited to the monosaccharides, the disaccharides and the oligosaccharides. In one aspect, a heterocycloalkyl is a C₂-C₁₀heterocycloalkyl. In another aspect, a heterocycloalkyl is a C₄-C₁₀heterocycloalkyl. In some embodiments, a heterocycloalkyl contains 0-2 N atoms in the ring. In some embodiments, a heterocycloalkyl contains 0-2 N atoms, 0-2 O atoms and 0-1 S atoms in the ring.

The term “bond” or “single bond” refers to a chemical bond between two atoms, or two moieties when the atoms joined by the bond are considered to be part of larger substructure. In one aspect, when a group described herein is a bond, the referenced group is absent thereby allowing a bond to be formed between the remaining identified groups.

The term “moiety” refers to a specific segment or functional group of a molecule. Chemical moieties are often recognized chemical entities embedded in or appended to a molecule.

The term “optionally substituted” or “substituted” means that the referenced group is optionally substituted with one or more additional group(s) individually and independently selected from halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from halogen, —CN, —NH₂, —NH(CH₃), —N(CH₃)₂, —OH, —CO₂H, —CO₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —SC₁—C₄alkyl, —S(═O)C₁-C₄alkyl, and —S(═O)₂C₁-C₄alkyl. In some embodiments, optional substituents are independently selected from halogen, —CN, —NH₂, —OH, —NH(CH₃), —N(CH₃)₂, —CH₃, —CH₂CH₃, —CF₃, —CH₂CF₃, —OCH₃, —OCH₂CH₃, —OCF₃ and —OCH₂CF₃. In some embodiments, substituted groups are substituted with one or two of the preceding groups. In some embodiments, an optional substituent on an aliphatic carbon atom (acyclic or cyclic) includes oxo (═O).

The term “acceptable” with respect to a formulation, composition or ingredient, as used herein, means having no persistent detrimental effect on the general health of the subject being treated.

The term “modulate” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator” as used herein, refers to a molecule that interacts with a target either directly or indirectly. The interactions include, but are not limited to, the interactions of an agonist, partial agonist, an inverse agonist, antagonist, degrader, or combinations thereof. In some embodiments, a modulator is an antagonist. In some embodiments, a modulator is a degrader.

The terms “administer,” “administering”, “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the compounds and methods described herein. In some embodiments, the compounds and compositions described herein are administered orally.

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered, which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is optionally determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound described herein, or a pharmaceutically acceptable salt thereof, and a co-agent, are both administered to a patient simultaneously in the form of a single entity or dosage. The term “non-fixed combination” means that the active ingredients, e.g. a compound described herein, or a pharmaceutically acceptable salt thereof, and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The terms “kit” and “article of manufacture” are used as synonyms.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### Albumin

Human serum albumin (HSA) is the most abundant protein in human blood plasma. It is produced in the liver and constitutes about half of the blood serum protein. It is soluble and monomeric. The amino acid sequence of HSA contains a total of 17 disulphide bridges, one free thiol (Cys 34), and a single tryptophan (Trp 214).

Human serum albumin (HSA) has multiple hydrophobic binding sites (a total of eight for fatty acids, an endogenous ligand of HSA) and binds a diverse set of drugs, especially neutral and negatively charged hydrophobic compounds. Two high affinity binding sites have been proposed in subdomains IIA and IIIA of HSA, which are highly elongated hydrophobic pockets with charged lysine and arginine residues near the surface which function as attachment points for polar ligand features.

In some embodiments, binding of drugs to albumin reduce the off-target toxicities associated with the free drug.

Albumin accumulates in malignant and inflamed tissue due to a leaky capillary combined with an absent or defective lymphatic drainage system.

Albumin reversibly binds to and transports a wide range of molecules, including bilirubin, free fatty acids, hydrophobic vitamins, hormones, calcium and zinc, as well as many acidic and hydrophobic drugs. Human serum albumin constitutes approximately 60% of total plasma protein and is the most important drug carrier protein in plasma on account of its high abundance. Albumin can facilitate the diffusion of lipophilic drugs into the membrane lipid bilayer. In addition, various proliferating tumors are known to accumulate albumin and use it as a major energy and nitrogen source for de novo protein synthesis.

As described herein, albumin is used as a drug carrier. In some embodiments, albumin is used a carrier for the DUB inhibitor compounds described herein. In some embodiments, described herein are methods for enhancing transport and enhancing binding of a DUB inhibitor compound to a tumor cell. Tumor cells exhibit an enhanced uptake of proteins including, for example, albumin and transferrin, as compared to normal cells. Since tumor cells are dividing at a rapid rate, they require additional nutrient sources compared to normal cells. In some embodiments, pharmaceutical compositions containing a DUB inhibitor compound and human serum albumin will show high uptake of the albumin-DUB inhibitor compound into tumors. In some embodiments, the high uptake of albumin-DUB inhibitor compound into tumors is due to the phenomenon of the albumin-drug transport by glycoprotein 60 (“gp60”) receptors, which are specific for albumin.

The transcytosis of albumin across the endothelium of blood vessels is mediated by gp60 and caveolae. Gp60 (albondin) is a 60-kDa glycoprotein localised on the endothelial cell surface that binds to native albumin with a high affinity in the nanomolar range. The binding of albumin to gp60 induces gp60 clustering and association with caveolar-scaffolding protein caveolin-1, which leads to the formation of vesicles called caveolae that carry both gp60-bound and fluid phase albumin or albumin-bound drugs in a process known as transcytosis from the apical to the basal membrane, where the vesicle contents are released into the sub-endothelial space.

The accumulation of albumin and albumin-bound drugs in the tumour interstitium is further facilitated by SPARC (Secreted Protein, Acidic and Rich in Cysteine). SPARC, a secreted glycoprotein also referred to as osteonectin and BM 40, has been identified as an albumin-binding protein. SPARC binding to albumin is saturable and specific and plays an important role in the increased tumour accumulation of albumin-bound drugs. Over-expression of SPARC in multiple tumour types (including but not limited to breast, prostate, oesophagus, gastric, colorectal, liver, lung, kidney, skin melanoma, bladder, head and neck, thyroid and brain tumours such as glioma, invasive meningioma, astrocytoma) is associated with increased tumour invasion, metastasis and poor prognosis.

Patients with active rheumatoid arthritis (RA) frequently develop hypoalbuminemia that is primarily caused by high albumin consumption at sites of inflammation. The metabolism of synovial cells is highly up-regulated, and uptake of albumin is probably a relevant source covering their high demand for nitrogen and energy. The permeability of the blood-joint barrier for albumin in inflamed joints of RA patients is markedly increased. In some embodiments, albumin is used as a carrier to deliver a DUB inhibitor to inflamed joints.

The HSA can be a naturally-occurring HSA or a synthetic HSA protein. Synthetic HSA proteins can have the same primary amino acid structure as naturally-occurring HSA, or they can be engineered to have a different amino acid structure (so long as normal HSA activities are substantially preserved; i.e., so long as the HSA exhibits the biochemical and biological properties of HSA).

For in vivo applications in another animal, an albumin that occurs naturally in that other animal can be used. For in vitro application, the immune response of an animal against the albumin of a different animal need not be considered, and substantially any albumin can be used. Thus, while the compositions and methods described herein have been discussed primarily in the context of using HSA, substantially the same compositions and methods can be made and performed for in vitro uses using albumins obtained from other animals, such as bovine serum albumin.

In some embodiments, HSA is obtained from a natural source (e.g., from blood or plasma harvested from a human donor). HSA can also be obtained from an organism (e.g., a bacterium, a fungus, a plant cell culture, or an insect cell culture) that does not normally produce HSA, but has been engineered to do so. A characteristic of HSA obtained from natural sources is that the HSA so obtained can bind with fatty acids that occur in the organism or the medium in which it is produced. HSA saturated with fatty acids or lipids can exhibit a limited capacity to form a complex with the fatty acid-molecule complexes described herein. If the HSA with which a complex is to be formed has been obtained from a natural source, then it can be desirable to remove some or all of the fatty acids and lipids that are associated with the HSA prior to contacting the HSA with the fatty acid-molecule conjugate described herein. Such a process is commonly referred to as “defatting” the HSA, and substantially any known defatting procedure can be used. A non-limiting example of a suitable defatting procedure (one involving charcoal treatment) is described in Chen, 1967, J. Biol. Chem. 242:173-181.

A preparation of HSA obtained from a natural source can include as contaminants other molecules that naturally occur in the source. For example, HSA preparations obtained from an organism that produces the HSA can be contaminated with other proteins normally produced by the organism. Furthermore, if the HSA is obtained from a human, the HSA preparation can include as a contaminant one or more human pathogens. Thus, although human blood and other human body fluids can be convenient and economical sources of HSA, the risk of pathogenic contamination by the fluid donor can make HSA obtained from non-human sources (e.g., cultured plant or insect cells) a preferable reagent in the compositions and methods described herein.

In some embodiments, described herein is a method of improving the pharmacokinetic profile of a DUB inhibitor by the use of albumin as a carrier.

In some embodiments, described herein is the use of albumin as a carrier for a DUB inhibitor. In some embodiments, the DUB inhibitor is bound physically or covalently to HSA.

In some embodiments, the DUB inhibitor is entrapped in a colloidal drug delivery system. In some embodiments, the colloidal drug delivery system is albumin microspheres. In some embodiments, the DUB inhibitor is bound physically to HSA through non-covalent interactions. In some embodiments, HSA encapsulated nanoparticles of a DUB inhibitor are described. In other embodiments, a DUB inhibitor is conjugated with an albumin binding group and the modified DUB inhibitor is then complexed with HSA. In some embodiments, an albumin binding group is conjugated to the DUB inhibitor compound. In other embodiments, an albumin binding group is conjugated to the DUB inhibitor compound via a metabolizable group. In other embodiments, a fatty acid is conjugated to the DUB inhibitor compound. In other embodiments, a fatty acid is conjugated to the DUB inhibitor compound via a metabolizable group. In other embodiments, a fatty acid is conjugated to the DUB inhibitor compound via a metabolizable group. In some embodiments, the fatty acid group binds to HSA in a non-covalent manner. Metabolizable groups contemplated include those that are broken down under the slightly acidic or reducing environment present in many cells, thereby resulting in the release of the DUB inhibitor either extracellularly in the slightly acidic environment often present in tumor tissue or intracellularly in the acidic and/or reducing endosomal or lysosomal compartments after cellular uptake of the albumin DUB inhibitor complex by the tumor cell.

Non-Covalent Albumin Complexes

In some embodiments, the DUB inhibitor described herein is contacted with HSA to form HSA encapsulated nanoparticles of the DUB inhibitor. Techniques for the formation of HSA encapsulation of therapeutic agents are known. In some embodiments, the nanoparticle albumin bound (Nab™) technology developed by Abraxis BioScience is used to form HSA encapsulated nanoparticles of the DUB inhibitor. In some embodiments, nanoparticles can be formed by pretreating the HSA with a modifying reagent. In some embodiments, the pretreatment of HSA is with a modifying reagent such as β-mercaptoethanol to break disulfide bonds and expose the hydrophobic region of HSA thus allowing drug binding to the hydrophobic region (c.f. Gong et al, Biomacromol. 2012, 13, p 23).

In some embodiments, the following general procedure is used in the preparation of HSA encapsulated nanoparticles of the DUB inhibitor.

A DUB inhibitor is dissolved in a suitable solvent (e.g., chloroform, methylene chloride, ethyl acetate, ethanol, tetrahydrofuran, dioxane, acetonitrile, acetone, dimethyl sulfoxide, dimethyl formamide, methyl pyrrolidinone, or the like, as well as mixtures of any two or more thereof). Next, human serum albumin is added (into the aqueous phase) to act as a stabilizing agent for the formation of stable nanodroplets. HSA is added at a concentration in the range of about 0.05 to 25% (w/v). In some embodiments, HSA is added at a concentration in the range of about 0.5 to 5% (w/v). Next, an emulsion is formed by homogenization under high pressure and high shear forces. In some embodiments, such homogenization is conveniently carried out in a high pressure homogenizer, typically operated at pressures in the range of about 3,000 up to 30,000 psi. In some embodiments, such processes are carried out at pressures in the range of about 6,000 up to 25,000 psi. The resulting emulsion comprises very small nanodroplets of the nonaqueous solvent (containing the dissolved DUB inhibitor) and very small nanodroplets of the protein stabilizing agent. Acceptable methods of homogenization include processes imparting high shear and cavitation such as high pressure homogenization, high shear mixers, sonication, high shear impellers, and the like. Finally, the solvent is evaporated under reduced pressure to yield a colloidal system composed of protein coated nanoparticles of pharmacologically active agent and protein. Acceptable methods of evaporation include the use of rotary evaporators, falling film evaporators, spray driers, freeze driers, lyophilization, and the like.

Following evaporation of solvent, the liquid suspension may be dried to obtain a powder containing the pharmacologically active agent and protein. The resulting powder can be redispersed at any convenient time into a suitable aqueous medium such as saline, bulfered saline, water, bulfered aqueous media, solutions of amino acids, solutions of vitamins, solutions of carbohydrates, or the like, as well as combinations of any two or more thereof, to obtain a suspension that can be administered to mammals. Methods contemplated for obtaining this powder include freeze drying, spray drying, lyophilization, and the like.

In some embodiments, there is provided a method for the formation of unusually small submicron particles (nanoparticles), i.e., particles which are less than 200 nanometers in diameter. In some embodiments, such particles are capable of being sterile-filtered before use in the form of a liquid suspension.

In order to obtain sterile-filterable particles (i.e., particles less than or equal to 200 nm), the DUB inhibitor is initially dissolved in a substantially water immiscible organic solvent (e.g., a solvent having less than about 5% solubility in water, such as, for example, methylene chloride or chloroform) at high concentration, thereby forming an oil phase containing the pharmacologically active agent. Suitable solvents are set forth above.

The oil phase employed in the process contains only the pharmacologically active agent dissolved in solvent. Next, a water miscible organic solvent (e.g., a solvent having greater than about 10% solubility in water, such as, for example, ethanol) is added to the oil phase at a final concentration in the range of about 1%-99% v/v or in the range of about 5%-25% v/v of the total organic phase. The water miscible organic solvent can be selected from such solvents as ethyl acetate, ethanol, tetrahydrofuran, dioxane, acetonitrile, acetone, dimethyl sulfoxide, dimethyl formamide, methyl pyrrolidinone, and the like. Alternatively, the mixture of water immiscible solvent with the water miscible solvent is prepared first, followed by dissolution of the pharmaceutically active agent in the mixture.

Next, human serum albumin is dissolved in aqueous media. This component acts as a stabilizing agent for the formation of stable nanodroplets. Optionally, a sulficient amount of the first organic solvent (e. g. chloroform) is dissolved in the aqueous phase to bring it close to the saturation concentration. A separate measured amount of the organic phase (which now contains the pharmacologically active agent, the first organic solvent and the second organic solvent) is added to the saturated aqueous phase, so that the phase fraction of the organic phase is between about 0.5%-015% v/v or between 1% and 8% v/v.

Next, a mixture composed of micro and nanodroplets is formed by homogenization at low shear forces. This can be accomplished in a variety of ways, employing, for example, a conventional laboratory homogenizer operated in the range of about 2,000 up to about 15,000 rpm. This is followed by homogenization under high pressure (i.e., in the range of about 3,000 up to 30,000 psi). The resulting mixture comprises an aqueous protein solution (e.g., human serum albumin), the DUB inhibitor, the first solvent and the second solvent. Finally, solvent is rapidly evaporated under vacuum to yield a colloidal dispersion system (pharmacologically active agent and protein) in the form of extremely small nanoparticles (i.e., particles in the range of about 10 nm-200 nm diameter), and thus can be sterile-filtered. In some embodiments, the size range of the particles is between about 50 nm-170 nm, depending on the formulation and operational parameters. Colloidal systems prepared in this manner may be further converted into powder form by removal of the water therefrom, e.g., by lyophilization at a suitable temperature-time profile. The protein (e.g., human serum albumin) itself acts as a cryoprotectant, and the powder is easily reconstituted by addition of water, saline or buffer, without the need to use such conventional cryoprotectants as mannitol, sucrose, glycine, and the like. While not required, it is of course understood that conventional cryoprotectants may be added to invention formulations if so desired.

In some embodiments, HSA encapsulated nanoparticles of the DUB inhibitor have a cross-sectional diameter of no greater than about 10 microns, no greater than about 9 microns, no greater than about 8 microns, no greater than about 7 microns, no greater than about 6 microns, no greater than about 5 microns, no greater than about 4 microns, no greater than about 3 microns, no greater than about 2 microns, or no greater than about 1 microns. In some embodiments, HSA encapsulated nanoparticles of the DUB inhibitor have a cross-sectional diameter of less than 1 micron.

In some embodiments, pharmaceutical compositions prepared with the HSA encapsulated nanoparticles of the DUB inhibitor include other agents, excipients, or stabilizers to improve properties of the composition. For example, to increase stability by increasing the negative zeta potential of nanoparticles or nanodroplets, certain negatively charged components may be added. Such negatively charged components include, but are not limited to bile salts of bile acids; phospholipids and negatively charged surfactants of emulsifiers.

The pharmaceutical composition can be stabilized with a pharmaceutically acceptable surfactant. The term “surfactants,” as used herein, refers to surface active group(s) of amphiphile molecules. Surfactants can be anionic, cationic, nonionic, and zwitterionic. Any suitable surfactant can be included in the inventive pharmaceutical composition. Examples of suitable surfactants are described in, for example, Becher, Emulsions: Theory and Practice, Robert E. Krieger Publishing, Malabar, Fla. (1965).

There are a wide variety of suitable formulations (see, e.g., U.S. Pat. No. 5,916,596). The following formulations and methods are merely exemplary and are in no way limiting. Formulations suitable for oral administration can consist of (a) liquid solutions, such as an effective amount of the compound dissolved in diluents, such as water or saline; (b) capsules, sachets or tablets, each containing a predetermined amount of the active ingredient, as solids or granules, (c) suspensions in an appropriate liquid, and (d) suitable emulsions.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described. In some embodiments, formulations are prepared as injectable formulations.

Formulations suitable for aerosol administration comprise the inventive pharmaceutical composition include aqueous and non-aqueous, isotonic sterile solutions, which can contain anti-oxidants, buffers, bacteriostats, and solutes, as well as aqueous and non-aqueous sterile suspensions that can include suspending agents, solubilizers, thickening agents, stabilizers, and preservatives, alone or in combination with other suitable components, which can be made into aerosol formulations to be administered via inhalation. These aerosol formulations can be placed into pressurized acceptable propellants, such as dichlorodifluoromethane, propane, nitrogen, and the like. They also can be formulated as pharmaceuticals for non-pressured preparations, such as in a nebulizer or an atomizer.

In some embodiments, the pharmaceutical composition is formulated to have a pH range of 4.5 to 9.0 or 5.0 to 8.0. The pharmaceutical composition can also be made to be isotonic with blood by the addition of a suitable tonicity modifier, such as glycerol. The pharmaceutically acceptable carrier comprises pyrogen-free water or water for injection, USP. In some embodiments, the pharmaceutical composition is prepared as a sterile aqueous formulation, a nanoparticle, an oil-in-water emulsion, or a water-in-oil emulsion. In some embodiments, the pharmaceutical composition is an oil-in-water emulsion.

In some embodiments, the pharmaceutical composition has a particle or droplet size less than about 200 nanometers (nm).

The dose of the pharmaceutical composition administered to a human will vary with the particular pharmaceutical composition, the method of administration, and the particular site being treated. The dose should be sufficient to affect a desirable response, such as a therapeutic or prophylactic response against a particular disease or condition.

In some embodiments, pharmaceutical compositions described herein are administered to the human via intravenous administration, intra-arterial administration, intrapulmonary administration, oral administration, inhalation, intravesicular administration, intramuscular administration, intra-tracheal administration, subcutaneous administration, intraocular administration, intrathecal administration, or transdermal administration. For example, in some embodiments, HSA encapsulated DUB inhibitors are administered by inhalation to treat conditions of the respiratory tract. There are minimal side-effects associated with the inhalation of the HSA encapsulated DUB inhibitors, as albumin is a natural component in the lining and secretions of the respiratory tract. In some embodiments, HSA encapsulated DUB inhibitors are used to treat respiratory conditions such as pulmonary fibrosis, broncheolitis obliterans, lung cancer, bronchoalveolar carcinoma, and the like.

In some embodiments, described herein is a method for enhancing transport of a DUB inhibitor to a desired area of a desired target tissue of a body, which method comprises administering to a human a pharmaceutical composition comprising a DUB inhibitor and a pharmaceutically acceptable carrier, wherein the pharmaceutically acceptable carrier comprises albumin, and wherein the ratio of albumin to the DUB inhibitor in the pharmaceutical composition is about 18:1 or less. In some embodiments, described herein is a method for enhancing binding of a DUB inhibitor to a cell in vitro or in vivo, which method comprises administering to said cell in vitro or in vivo a pharmaceutical composition comprising a DUB inhibitor and a pharmaceutically acceptable carrier, wherein the pharmaceutically acceptable carrier comprises albumin, and wherein the ratio of albumin to pharmaceutical agent in the pharmaceutical composition is about 18:1 or less.

In the methods for enhancing transport of a pharmaceutical agent (e.g. a DUB inhibitor) to a target tissue (e.g. a tumor) or for enhancing binding of a pharmaceutical agent to a cell, the pharmaceutically acceptable carrier preferably comprises albumin, most preferably human serum albumin. Not to adhere to any one particular theory, it is believed that the ratio of protein, e.g., human serum albumin, to pharmaceutical agent in the pharmaceutical composition affects the ability of the pharmaceutical agent to bind and transport the pharmaceutical agent to a cell. In this regard, higher ratios of protein to pharmaceutical agent generally are associated with poor cell binding and transport of the pharmaceutical agent, which possibly is the result of competition for receptors at the cell surface. The ratio of protein, e.g., albumin, to active pharmaceutical agent must be such that a sufficient amount of pharmaceutical agent binds to, or is transported by, the cell. Exemplary ranges for protein-drug preparations are protein to drug ratios (w/w) of 0.01:1 to about 100:1, or 0.02:1 to about 40:1. While the ratio of protein to pharmaceutical agent will have to be optimized for different protein and pharmaceutical agent combinations, generally the ratio of protein, e.g., albumin, to pharmaceutical agent is about 18:1 or less (e.g., about 15:1, about 10:1, about 5:1, or about 3:1).

The amount of albumin included in the pharmaceutical composition will vary depending on the pharmaceutical active agent, other excipients, and the route and site of intended administration.

Typically, the pharmaceutical composition is prepared in liquid form, and the albumin is then added in solution. In some embodiments, the pharmaceutical composition is in liquid form and comprises from about 0.1 percent to about 25 percent by weight (e.g. about 0.5 percent by weight, about 5 percent by weight, about 10 percent by weight, about 15 percent by weight, or about 20 percent by weight) of albumin. The pharmaceutical composition can be dehydrated, for example, by lyophilization, spray-drying, fluidized-bed drying, wet granulation, and other suitable methods known in the art. In some embodiments, the composition is prepared in solid form, such as by wet granulation, fluidized-bed drying, and other methods known in the art. In some embodiments, the composition is prepared in solid form, such as by wet granulation, fluidized-bed drying, and other methods known in the art and the albumin is applied to the active pharmaceutical agent, and other excipients if present, as a solution.

In some embodiments, described herein is a method for enhancing binding of a pharmaceutical agent (e.g. a DUB inhibitor) to a cell in vitro or in vivo, wherein the pharmaceutical composition is administered to a cell in vitro or in vivo. In some embodiments, the cell is a mammalian cell. In some embodiments, the cell is a human cell. In some embodiments, the pharmaceutical composition is administered to a cell in vivo. The cell can be any suitable cell that is a desirable target for administration of the pharmaceutical composition. In some embodiments, the cell is located in or derived from tissues of the digestive system including, for example, the esophagus, stomach, small intestine, colon, rectum, anus, liver, gall bladder, and pancreas. In some embodiments, the cell also is located in or derived from tissues of the respiratory system, including, for example, the larynx, lung, and bronchus. In some embodiments, the cell is located in or derived from, for example, the uterine cervix, the uterine corpus, the ovary vulva, the vagina, the prostate, the testis, and the penis, which make up the male and female genital systems, and the urinary bladder, kidney, renal pelvis, and ureter, which comprise the urinary system. In some embodiments, the cell is located in or derived from tissues of the cardiovascular system, including, for example, endothelial cells and cardiac muscle cells. In some embodiments, the cell is located in or derived from tissues of the lymphoid system (e.g., lymph cells), the nervous system (e.g., neurons or glial cells), and the endocrine system (e.g., thyroid cells). In some embodiments, the cell is located in or derived from tissues of the cardiovascular system. In some embodiments, the cell is an endothelial cell. In some embodiments, the pharmaceutical composition contacts more than one cell.

In one aspect, described herein are methods for enhancing transport and enhancing binding of a pharmaceutical agent (e.g. a DUB inhibitor) to a tumor cell in a mammal. In these embodiments, the methods are used to treat cancer. Tumor cells exhibit an enhanced uptake of proteins including, for example, albumin and transferrin, as compared to normal cells. Since tumor cells are dividing at a rapid rate, they require additional nutrient sources compared to normal cells.

Conjugation with an Albumin Binding Group

In some embodiments, a DUB inhibitor is conjugated with an albumin binding group and the conjugated compound is complexed with HSA. In some embodiments, the conjugated compound is complexed with HSA using the nab technology described above.

In some embodiments, described herein is a compound with the following Formula B:

(DUB)-L-Ab Formula B

wherein,
DUB is a deubiquitinase inhibitor;
L is a absent or a linker;
Ab is an albumin binding group.

The term “albumin binding group” as used herein means a group which binds non-covalently or covalently to human serum albumin. The albumin binding group attached to the DUB inhibitor has a high affinity to human serum albumin. A range of albumin binding groups that bind non-coavlently to HSA are known, such as, but not limited to: fatty acids (e.g. linear and branched lipohophilic moieties containing 4-40 carbon atoms), steroids, thyroid hormones, compounds with a cyclopentanophenanthrene skeleton, peptides having 10-30 amino acid residues, etc.

In some embodiments, the albumin binding group is attached covalently. Suitable protein binding groups are capable of covalently binding to an amino, guanidine, hydroxyl or thiol group of HSA.

In some embodiments, the albumin binding group is an alkyl acid. In some embodiments, the alkyl acid is a saturated alkyl acid or an unsaturated alkyl acid.

Saturated alkyl acids include but are not limited to: butanoic acid (butyric acid), pentanoic acid (valeric acid), hexanoic acid (caproic acid), heptanoic acid (enanthic acid), octanoic acid (caprylic acid), nonanoic acid (pelargonic acid), decanoic acid (capric acid), undecanoic acid (undecylic acid), dodecanoic acid (lauric acid), tridecanoic acid (tridecylic acid), tetradecanoic acid (myristic acid), pentadecanoic acid (pentadecylic acid), hexadecanoic acid (palmitic acid), heptadecanoic acid (margaric acid), octadecanoic acid (stearic acid), nonadecanoic acid (nonadecylic acid), eicosanoic acid (arachidic acid), heneicosanoic acid, docosanoic acid (behenic acid), tricosanoic acid, tetracosanoic acid (lignoceric acid), pentacosanoic acid, hexaconsanoic acid (cerotic acid), heptacosanoic acid, octacosanoic acid (montanic acid), nonacosanoic acid, triacontanoic acid (melissic acid), hentriacontanoic acid. In some embodiments, the albumin binding group is myristic acid.

Unsaturated alkyl acids include but are not limited to: 10-undecenoic acid (10-undecylenic acid), cis-9-tetradecenoic acid (myristoleic acid), trans-9-tetradecenoic acid (myristelaidic acid), 9-tetradecynoic acid, cis-10-pentadecenoic acid cis-9-hexadecenoic acid (palmitoleic acid), trans-9-hexadecenoic acid (palmitelaidic acid), cis-10-heptadecenoic acid, cis-6-octadecenoic acid (petroselinic acid), trans-6-octadecenoic acid, cis-7-octadecenoic acid, trans-7-octadecenoic acid, cis-9-octadecenoic acid (oleic acid), trans-9-octadecenoic acid (elaidic acid), 9-octadecynoic acid (stearolic acid), cis-11-octadecenoic acid (cis-vaccenic acid), trans-11-octadecenoic acid (trans-vaccenic acid), cis-12-octadecenoic acid, trans-12-octadecenoic acid, cis-13-octadecenoic acid, trans-13-octadecenoic acid, cis-15-octadecenoic acid, 17-octadecynoic acid, cis-12-hydroxy-9-octadecenoic acid (ricinoleic acid), trans-12-hydroxy-9-octadecenoic acid (ricinelaidic acid), cis-9, 12-octadecadienoic acid (linoleic acid), trans-9, 12-octadecadienoic acid (linolenelaidic acid), 9, 11(10, 12)-octadecadienoic acid, cis-6, 9, 12-octadecatrienoic acid (γ-linolenic acid), cis-9, 12, 15-octadecatrienoic acid (linolenic acid), trans-9, 12, 15-octadecatrienoic acid (linolenelaidic acid), cis-6, 9, 12, 15-octadecatetraenoic acid, cis-10-nonadecenoic acid, cis-5-eicosenoic acid, cis-8-eicosenoic acid, cis-11-eicosenoic acid (gondoic acid), trans-11-eicosenoic acid, cis-13-eicosenoic acid, 13-eicosynoic acid, cis-11, 14-eicosadienoic acid, cis-5, 8, 11-eicosatrienoic acid, 5, 8, 11-eicosatriynoic acid, cis-8, 11, 14-eicosatrienoic acid, 8, 11, 14-eicosatriynoic acid, cis-11, 14, 17-eicosatrienoic acid, cis-5, 8, 11, 14-eicosatetraenoic acid (arachidonic acid), cis-5, 8, 11, 14, 17-eicosapentaenoic acid, cis-13-docosenoic acid (erucic acid), trans-13-docosenoic acid (brassidic acid), cis-13, 16-docosadienoic acid, cis-13, 16, 19-docosatrienoic acid, cis-7, 10, 13, 16-docosatetraenoic acid, cis-7, 10, 13, 16, 19-docosapentaenoic acid, cis-4, 7, 10, 13, 16, 19-docosahexaenoic acid, cis-15-tetracosenoic acid (nervonic acid).

In some embodiments, the albumin binding group is a long chain fatty acid. Long chain fatty acids (LCFAs, i.e., carboxylic acids having an aliphatic chain with 10-36 carbon atoms in its backbone, either saturated or unsaturated) are essential for many cellular functions. Fatty acids are important sources of fuel because, when metabolized, they yield large quantities of ATP. LCFAs serve as an important energy resource and are also critical components of lipids, hormones, and proteins. LCFAs are known to be bound and transported by HSAs within the human body.

The chemical means or bonds by which the DUB inhibitor is conjugated with the fatty acid are not critical. In one embodiment, the conjugation of the molecule and fatty acid is selected on the basis of being reversible upon uptake of the conjugate within a cell (i.e., the conjugating linkage is physiologically reversible). By way of example, the reducing environment present in many cells can result in cleavage of a disulfide bond between a molecule (i.e. DUB inhibitor) and a thiol-substituted fatty acid, resulting in generation of free molecule and free fatty acid upon uptake of the conjugate into a cell. Physiological reversibility of the conjugation is not necessary unless the conjugation prevents the molecule from exhibiting its desired function. By contrast, conjugation that limits or eliminates a property of the molecule to be delivered can be beneficial for preventing inappropriate exhibition of that property prior to cellular uptake.

In some embodiments, fatty acids are conjugated to the DUB inhibitor using standard peptide coupling chemistry to form amide or ester bonds.

Details of reagents and reaction conditions not specifically described herein are readily determinable by a skilled artisan in this field.

In some embodiments, described herein is a compound of Formula B-1:

(DUB)-L-X—Y-(Alk) Formula B-1

wherein
DUB is a deubiquitinase inhibitor described herein;
L is a absent or a linker;
X is absent, —O—, —S— or —NH—;
Y is —C(═O)— or —SO₂—;
Alk is C₆-C₄₀alkyl.

In some embodiments, L is absent. In some embodiments, L is a metabolizable linker. In some embodiments, L is —CH═N—, —CH═N—NH—, —C(alkyl)=N—, or —C(alkyl)=N—NH—. In some embodiments, L is —CH═N—. In some embodiments, L is —C(alkyl)=N—. In some embodiments, L is —S—S—.

In some embodiments, Y is —C(═O)—.

In some embodiments, Alk is C₆alkyl, C₇alkyl, C₈alkyl, C₉alkyl, C₁₀alkyl, C₁₁alkyl, C₁₂alkyl, C₁₃alkyl, C₁₄alkyl, C₁₅alkyl, C₁₆alkyl, C₁₇alkyl, C₁₈alkyl, C₁₉alkyl, C₂₀alkyl, C₂₁alkyl, C₂₂alkyl, C₂₃alkyl, C₂₄alkyl, C₂₅alkyl, C₂₆alkyl, C₂₇alkyl, C₂₈alkyl, C₂₉alkyl, C₃₀alkyl, C₃₁alkyl, C₃₂alkyl, C₃₃alkyl, C₃₄alkyl, C₂₅alkyl, C₃₆alkyl, C₃₇alkyl, C₃₈alkyl, C₃₉alkyl, or C₄₀alkyl. In some embodiments, Alk is C₁₃alkyl.

In some embodiments, Alk is —(CH₂)₅—CH₃, —(CH₂)₆—CH₃, —(CH₂)₇—CH₃, —(CH₂)₈—CH₃, —(CH₂)₉—CH₃, —(CH₂)₁₀—CH₃, —(CH₂)₁₁—CH₃, —(CH₂)₁₂—CH₃, —(CH₂)₁₃—CH₃, —(CH₂)₁₄—CH₃, —(CH₂)₁₅—CH₃, —(CH₂)₁₆—CH₃, —(CH₂)₁₇—CH₃, —(CH₂)₁₈—CH₃, —(CH₂)₁₉—CH₃, —(CH₂)₂₀—CH₃, —(CH₂)₂₁—CH₃, —(CH₂)₂₂—CH₃, —(CH₂)₂₃—CH₃, —(CH₂)₂₄—CH₃, —(CH₂)₂₅—CH₃, —(CH₂)₂₆—CH₃, —(CH₂)₂₇—CH₃, —(CH₂)₂₈—CH₃, —(CH₂)₂₉—CH₃, —(CH₂)₃₀—CH₃, —(CH₂)₃₁—CH₃, —(CH₂)₃₂—CH₃, —(CH₂)₃₃—CH₃, —(CH₂)₃₄—CH₃, —(CH₂)₃₅—CH₃, —(CH₂)₃₆—CH₃, —(CH₂)₃₇—CH₃, —(CH₂)₃₈—CH₃, or —(CH₂)₃₉—CH₃. In some embodiments, Alk is —(CH₂)₁₂—CH₃.

In some embodiments, L is absent.

In some embodiments, L is a linker that connects the albumin binding group and the DUB inhibitor. In some embodiments, L is a hydrophobic linker. In some other embodiments, L is a hydrophilic linker. In some embodiments, the linker is linear. In other embodiments, the linker is branched. In some embodiments, a branched linker allows for conjugation to more than one albumin binding group. In some embodiments, a branched linker allows for conjugation to another bioactive molecule. In some embodiments, a branched linker allows for conjugation to cell-penetrating peptides (CPPs) to enhance cell penetration.

Linker length may be viewed in terms of the number of linear atoms between the DUB inhibitor and the albumin binding group, with cyclic moieties to be counted by taking the shortest route around the ring. In some embodiments, the linker has a linear stretch of between 1-30 atoms, in other embodiments 1-20 atoms, in still other embodiments 1-15 atoms, in still other embodiments 1-10 atoms, and in still other embodiments 1-5 atoms. In some embodiments, the length of the linker is a range with a lower limit selected from the group consisting of 1, 2, 3, 4, 5, 6, 7, 8, 9, 10, and an upper limit selected from the group consisting of 5, 6, 7, 8, 9, 10, 11, 12, 13, 14, 15, 16, 17, 18, 19, 20, 25, and 30.

Other linker considerations include the effect on physical or pharmacokinetic properties of the resulting compound, such as solubility, lipophilicity, hydrophilicity, hydrophobicity, stability (more or less stable as well as planned degradation), rigidity, flexibility, immunogenicity, modulation of antibody binding, the ability to be incorporated into a micelle or liposome, and the like.

In some embodiments, the linker may be a peptidyl linker. In some embodiments, the peptidyl linker may be between 3-20 amino acids long, such as repeats of a single amino acid residue (e.g. polyglycine) or combinations of amino acid residues to give a peptide linker which imparts favorable presentation of the DUB inhibitor or pharmacokinetics.

Alternatively, the linker may be a non-peptidyl linker. Typical examples of these types of linker would be those based on straight or branched chain hydrocarbons or polyethylene glycols of varying lengths. These may incorporate other groups to effect solubility, rigidity, isoelectric point, such as aromatic or non-aromatic rings, halogens, ketones, aldehydes, esters, sulfonyls, phosphate groups, and so on.

In some embodiments, L is (CH₂)₀₋₁₀ or (CH₂CH₂O)₀₋₁₀.

In some embodiments, the L group is attached to the DUB inhibitor through a bond, oxygen, nitrogen, sulfur, sulfoxide, sulfone, methylene, ketone, amide, ester, urea, carbamate, carbonate, or such group.

In some embodiments, the compound of Formula B or B-1 has the following structure of Formula B-2:

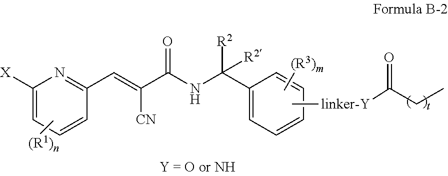

wherein t is 3, 4, 5, 6, 7, 8, 9, 10, 11 or 12; and the other variables are as described herein.

In some embodiments, t is 3. In some embodiments, t is 4. In some embodiments, t is 5. In some embodiments, t is 6. In some embodiments, t is 7. In some embodiments, t is 8. In some embodiments, t is 9. In some embodiments, t is 10. In some embodiments, t is 11. In some embodiments, t is 12.

In some embodiments, the compound of Formula B or B-1 or B-2 has the following structure of Formula B-3:

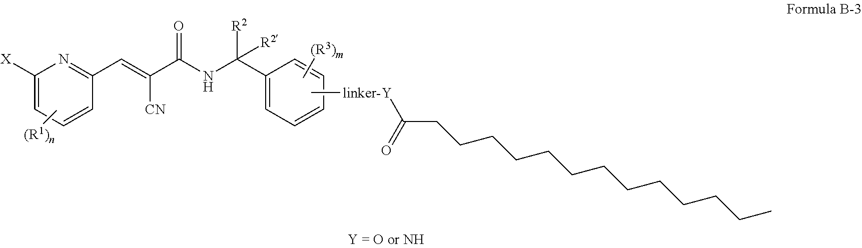

In some embodiments, formation of conjugates between LCFAs and DUB inhibitors described herein enhances the serum stability and delivery of the DUB inhibitor by a mechanism facilitated by binding of the small molecule-LCFA conjugate with HSA.

In another aspect, described herein is a method of enhancing delivery of a DUB inhibitor to a cell of a human. The method involves conjugating a DUB inhibitor with a fatty acid to form a conjugate. A first composition that includes the conjugate and a second composition that includes HSA are contacted outside the body of the human. The first and second compositions are contacted for a time and under conditions sufficient for the conjugate to bind with the albumin to form a complex. The complex is then administered to the human in a suitable manner, such as, but not limited to injection or infusion.

Uptake of long chain fatty acids (LCFAs) is an essential cellular metabolic process. It is known that cell are able to take up LCFAs by means of receptor-mediated binding and transmembrane transportation. LCFAs are also known to taken up by cells by means of passive diffusion. It is also known that cells can translocate LCFA across the cellular membrane by means of pathways mediated by albumin proteins that are capable of binding with LCFAs and translocating across cellular membranes.

Peptides conjugated to myristic acid are taken up by live cells thereby enhancing the cell penetration of the peptide (see Nelson et al, Myristoyl-Based transport of Peptides into Live Cells, Biochemistry 2009, 46 p 14771-14781). Myristoyl-based conjugates of biomolecules have been used to enhance cell penetration and improve antiproliferative effects of the parent biomolecule (c.f. Leonidova et al, ACS Med Chem Letts., 2014, accepted on line; and Sturzu et al, Amino Acids, 2009, 37, p 249-255). In some embodiments, enhanced cellular penetration is achieved by conjugation to cell-penetrating peptides (CPPs) or protein transduction domains (PTDs) (see Stephens and Pepperkok, The many ways to cross the plasma membrane, PNAS, 2001, 98, p 4295-4298).

Complexing HSA with the Fatty Acid-Conjugated Molecule

The target-binding albumins described herein can be used on their own, without a fatty acid-conjugated molecule complexed therewith. In important embodiments of the methods and compositions described herein, a fatty acid conjugated molecule is present in a complex wherein at least the fatty acid portion of the conjugate interacts with HSA. This complex is formed by contacting the conjugate with a composition that includes HSA. The conjugate and the HSA-containing composition are contacted for a time and under conditions sufficient for the conjugate to bind with the albumin to form a complex.

The duration and conditions under which the compositions need be contacted depend on the particular properties of the molecule, the fatty acid, and the HSA that are used, and these duration and condition parameters are readily determinable by a skilled artisan in this field.

The HSA with which the conjugate is to be complexed is preferably present in excess (e.g. 2- to 5-fold molar excess). The time required for approximately equilibrium binding between the HSA and the conjugate is dependent on the chemical species involved, the temperature, the degree and type of agitation, and other factors. In some embodiments, the duration for which complex formation is allotted is generally on the time scale of minutes to hours (i.e. 5 minutes to 12 hours). The goal of such contacting is to bring the conjugate into sufficient proximity to the HSA that the forces of molecular attraction between the HSA and the fatty acid portion of the conjugate are able to act and cause complex formation. If the HSA and the conjugate are both soluble in a solvent that does not significantly denature the HSA, then the HSA and the conjugate can simply be combined in the solvent, and stirring or other agitation need not be applied. If the conjugate is in a solvent that is substantially insoluble with the solvent in which the HSA is suspended, then an interface will exist between the two solvents, and complexation can occur substantially only at the interface. In such instances, procedures for contacting insoluble liquid phases (e.g., stirring, shaking, mixing, or emulsification) can be performed in order to increase the rate of complex formation. If such procedures are employed, care should be taken to avoid denaturing or otherwise damaging the HSA.

The compositions described herein can be purified to remove undesirable components at various stages, using any of the wide variety of purification technologies known in the art. By way of example, it can be undesirable to have reagents employed in the fatty acid-molecule conjugation reaction present when the conjugate is complexed with HSA. It can be preferable, therefore, to purify the conjugate from such reagents. In some embodiments, the purification removes all of the undesried species. In some other embodiments, the purification reduces the concentration of the undesired species.

Any of the wide variety of purification procedures (e.g., chromatography, electrophoresis, centrifugation) known in the art can be used to effect such purification. A quick, simple, and effective way of separating relatively large molecules from substantially smaller molecules is ultrafiltration, in which pressure is applied to a liquid mixture above a membrane having pores of sufficient size to substantially retard passage of the large molecules while permitting passage of small molecules. Ultrafiltration methods can be used to purify products described herein from substantially smaller molecular species (e.g., from conjugation reagents or from non-conjugated, non-complexed drug molecules).

Covalent Adducts

The DUB inhibitors described herein comprise an electrophilic Michael acceptor. In some embodiments, the free thiol (Cys 34) of HSA is reacted with the electrophilic Michael acceptor to form a covalent adduct:

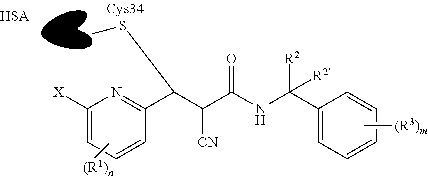

In some embodiments, the HSA-DUB inhibitor adduct is administered to a mammal. In some embodiments, uptake of the albumin-DUB inhibitor adduct into a target tissue (e.g. a tumor) is followed by a beta-elimination (i.e. retro-Michael reaction) of HSA thereby regenerating the electrophilic Michael acceptor moiety in situ. In some embodiments, the beta elimination is triggered by breakdown of the HSA within the target tissue (e.g. tumor).

In some embodiments, the free thiol (Cys 34) of HSA is covalently attached to the DUB inhibitor through a linker as depicted below:

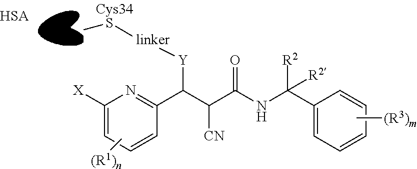

In some embodiments, Y is O, S, or NH. In some embodiments, the linker comprises a succinimide group. In some embodiments, the free thiol (Cys 34) of HSA is covalently attached to a succinimide group in the linker. In some embodiments, the spacing between the succinimide group and the Y group is variable in length and identity. In some embodiments, in addition to the succinimide group in the linker, other functional groups are present adding to the steric bulk of the linker and/or propensity of the adduct to undergo a retro-Michael reaction. In some embodiments, in addition to the succinimide group in the linker, other functional groups are present that result from the covalent attachment of the DUB inhibitor e.g. a triazole which is formed by a “click” reaction to join the HSA and DUB moieties together (c.f. Legigan et al, J. Med. Chem., 2012, 55, p 4516). In some embodiments, the linker is

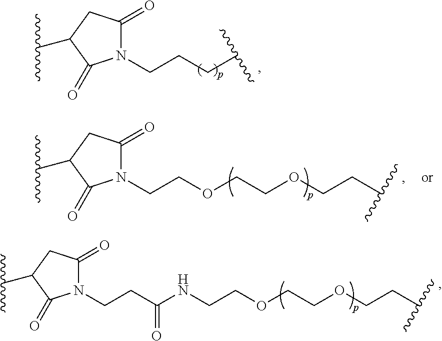

where p is 0, 1, 2, 3, or 4.

In some embodiments, the DUB inhibitor is attached to the —NH₂ of lysine residues or the guanidine group of arginine residues of HSA by reaction with coupling reagents. In some embodiments, the free NH₂ of HSA is covalently attached to the DUB inhibitor through a linker as depicted below:

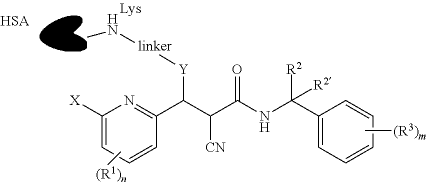

In some embodiments, Y is O, S, or NH. In some embodiments, the NH₂ of HSA is covalently attached via a carbonyl to form an amide group to the linker. In some embodiments, the NH₂ of HSA is covalently attached via a squaric acid group in the linker. In some embodiments, the spacing between the carbonyl group and the Y group is variable in length and identity. In some embodiments, in addition to the carbonyl group in the linker, other functional groups are present adding to the steric bulk of the linker and/or propensity of the adduct to undergo a retro-Michael reaction. In some embodiments, the linker is

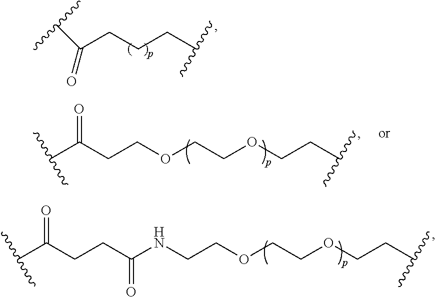

where p is 0, 1, 2, 3, or 4.

In some embodiments, a DUB inhibitor is prepared with a heterobifunctional cross-linking group. In some embodiments, a DUB inhibitor is prepared with a heterobifunctional cross-linker that enables covalent attachment of the functionalized DUB inhibitor to the free thiol of HSA. The heterobifunctional cross-linker comprises a thiol reactive group. Common thiol reactive groups include maleimides, halogens (e.g. haloacetyl), and pyridyl disulfides. Maleimides react specifically with free sulfhydryls (cysteine residues) in minutes, under slightly acidic to neutral (pH 6.5-7.5) conditions. Halogens (iodoacetyl functions) react with SH groups at physiological pH's. Both of these reactive groups result in the formation of stable thioether bonds.

There are a large number of chemical cross-linking agents that are known to those skilled in the art. Heterobifunctional cross-linkers provide the ability to design more specific coupling methods for conjugating to proteins, thereby reducing the occurrences of unwanted side reactions such as homo-protein polymers. A wide variety of heterobifunctional cross-linkers are known in the art. These include: succinimidyl 4-(N-maleimidomethyl)cyclohexane-1-carboxylate (SMCC), m-maleimidobenzoyl-N-hydroxysuccinimide ester (MBS); succinimidyl (4-iodoacetyl) aminobenzoate (SIAB), succinimidyl 4-(p-maleimidophenyl) butyrate (SMPB), 1-ethyl-3-(3-dimethylaminopropyl)carbodiimide hydrochloride (EDC); 4-succinimidyloxycarbonyl-methyl-(2-pyridyldithio)-toluene (SMPT), succinimidyl 3-(2-pyridyldithio)propionate (SPDP), succinimidyl 6-[3-(2-pyridyldithio)propionate]hexanoate (LC-SPDP). Those cross-linking agents having N-hydroxysuccinimide moieties can be obtained as the N-hydroxysulfosuccinimide analogs, which generally have greater water solubility. In addition, those cross-linking agents having disulfide bridges within the linking chain can be synthesized instead as the alkyl derivatives so as to reduce the amount of linker cleavage in vivo.

Albumin Binding Prodrugs and Conjugates

In some embodiments, the DUB inhibitor described herein is coupled to HSA using protein-binding group, wherein the protein binding group is covalently attached to the DUB inhibitor with or without a suitable linker. In some embodiments, described herein is a compound with the following Formula B:

(DUB)-L-Ab Formula B

wherein,
DUB is a deubiquitinase inhibitor;
L is a absent or a linker;
Ab is an albumin binding group.

In some embodiments, Ab is a protein binding group that covalently binds to human serum albumin. Suitable protein binding groups include those groups capable of covalently binding to an amino, guanidine, hydroxyl or thiol group of a protein (e.g. HSA). In some embodiments, the protein binding group covalently binds to the free thiol (Cys 34) of HSA. In some embodiments, the protein binding group is a maleimide group, a halogenacetamide group, a halogenacetyl group, a halogenacetate group, a pyrdinylthio group, a vinylcarbonyl group, an aziridine group, a disulfide group, an acetylene group, a hydroxysuccinimide group and the like. In some embodiments, the protein binding group is a maleimide group. Examples for maleimide protein binding groups are Kratz et al J. Med. Chem., 2000, 43, p 1253; Kratz et al J. Med. Chem., 2002, 45, p 5523 and Legigan J. Med. Chem., 201255, p 4516. In some embodiments, the protein binding group is a thiol group, allowing a disulfide bridge to be formed between the DUB inhibitor or linker and HSA.

In some embodiments, Ab is

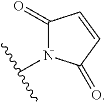 [SMILES: *N1C(=O)C=CC1=O]

In some embodiments, the linker is a cleavable linker. The term “cleavable linker” refers to any linker that can be cleaved physically or chemically. Examples for physical cleavage may be cleavage by light, radioactive emission or heat, while examples for chemical cleavage include cleavage by redox-reactions, hydrolysis, pH-dependent cleavage or cleavage by enzymes. Cleavage of the cleavable linker can be performed in vivo.

In some embodiments, the cleavable linker comprises one or more hydrolytically cleavable bonds, the hydrolysis of which releases the DUB inhibitor from the protein binding group. Examples of hydrolytically cleavable bonds are ester bonds or metal-complex bonds, such as are present in platinum-dicarboxylate complexes, where a diaminediaquoplatinum(II) complex is liberated.

In another preferred embodiment, the cleavable linker is cleavable by an enzyme. For example, the cleavable linker includes at least one peptide bond which preferably lies within a cleavable peptide sequence of a protease. A peptide bond can therefore be implemented by the insertion of a respective peptide sequence into the cleavable linker. Suitable enzymes are, for example, proteases and peptidases, e.g. matrix metalloproteases (MMP), cysteine proteases, serine proteases and plasmin activators, which are formed or activated in intensified manner in diseases such as rheumatoid arthritis or cancer, leading to excessive tissue degradation, inflammations and metastasis. Non-limiting examples of proteases include MMP-2, MMP-3 and MMP-9, cathepsin B, H, L and D, plasmin, urokinase, and prostate-specific antigen (PSA). In some embodiments, peptide sequences peptide sequences that are incorporated in the cleavable linker include: Arg, Arg-Arg, Phe-Arg, Phe-Cit, Ile-Pro Lys, Lys-Lys, Arg-Lys, Ala-Leu-Ala-Leu, Phe-Lys, Phe-Lys-Ala, Val-Cit, Val-Arg, Ala-Phe-Lys, D-Ala-Phe-Lys, Met, Met-Met, Phe-Met, Tyr-Met, Ala-Met, Ala-Phe-Met, Phe-Ala-Met, Ala-Tyr-Met, Phe-Tyr-Met, Ser-Ser-Tyr-Tyr-Ser-Arg, Phe-Pro-Lys-Phe-Phe-Ser-Arg-Gln, Lys-Pro-Ile-Glu-Phe-Nph-Arg-Leu, Gly-Pro-Leu-Gly-Ile-Ala-Gly-Gln, Gly-Pro-Leu-Gly-Ile-Ala-Gly-Gln, Gly-Pro-Gln-Gly-Ile-Trp-Gly-Gln, Gly-Phe-Leu-Gly. In addition, the enzymatically cleavable linker may contain a self-immolative linker such as a self-immolative p-aminobenzyloxycarbonyl (PABC) linker or a N-methyl- or symmetric N,N-dimethylethylene linker.

In some embodiments, the cleavable linker comprises at least one acid-labile bond. Examples of acid-labile bonds include esters, amides, acetals, ketals, imines, hydrazones, carboxylhydrazones and sulfonylhydrazone bonds and bonds containing a trityl group. In some embodiments, the cleavable linker is a carboxylic hydrazone linker.

In contrast to low-molecular weight compounds, the cellular uptake of macromolecules occurs not via diffusion through the cell membrane, but via endocytosis. During this process, the pH value decreases from initially 7.2-7.4 to about 6.5-5.0 in the endosomes. A subsequent conversion into primary or secondary lysosomes may even lead to a pH value of around 4. In some embodiments, the interstitial pH value in tumor tissue is generally 0.5-1.0 pH units lower compared to the respective pH value in healthy tissue. However, the cytosolic pH value of tumor cells generally does not differ from the pH value of other cells. Accordingly, said significant change in the pH value after cellular uptake in tumor tissue has been used to develop acid-labile bonds between the carrier and the drug. Examples for known acid-labile pre-determined breaking points are acetal bonds, cis-aconityl and trityl groups, and acyl hydrazones, and are described e.g. in F. Kratz, U. Beyer, M. T. Schütte, Crit. Rev. Ther. Drug Carrier Sys. 1999, 16, 245-288.

Cleavable linkers comprising acid-labile trigger groups provide the advantage that they can be used in a wide range of applications, since the decrease in the pH value is a characteristic feature of tumor tissue. To the contrary, cleavable linkers comprising trigger groups which are cleaved upon enzymatic activation can only be applied when there is an (over)expression of the target enzyme in the tumor entity to be treated.

The most commonly used acid-labile groups are acyl hydrazones. Specific acyl hydrazones have been developed that show significant differences between their stability at a pH 7 (t1/2>>48 h) and their stability at a pH 5 (t1/2<30 h), which is advantageous in view of their cleavage in tumor cells upon a decrease in the pH value. However, in order to couple a drug to a macromolecular carrier via an acid-labile hydrazone bond, it is necessary that the drug comprises either a carbonyl functionality or an acyl hydrazide group.

In a some embodiments, the cleavable linker comprises a substituted or unsubstituted, branched-chain or straight-chain aliphatic alkyl group with 1 to 20 carbon atoms, which may comprise one or more oxygen, sulfur and/or nitrogen atoms, and/or amide, ester or aryl residues.

In some embodiments, L is absent, alkylene, alkylene acid, a polyethylene glycol, polyethylene glycol acid, polyethylene glycol diacid, amino polyethylene glycol, amino polyethylene glycol acid, amino polyethylene glycol diacid or polyglycine. In some embodiments, L is absent,

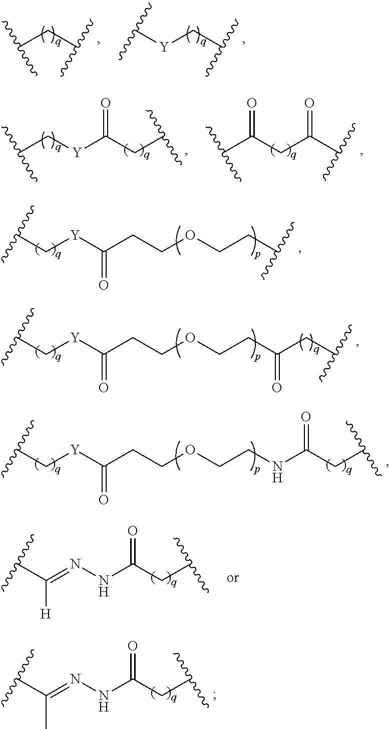

wherein Y is O or NH; each q is independently selected from 0, 1, 2, 3, 4, and 5; and p is 0, 1, 2, 3 or 4.

In some embodiments, each q is independently selected from 0, 1, 2, 3, and 4. In some embodiments, q is 0, 1, 2, 3, or 4.

In some embodiments, -L-Ab is:

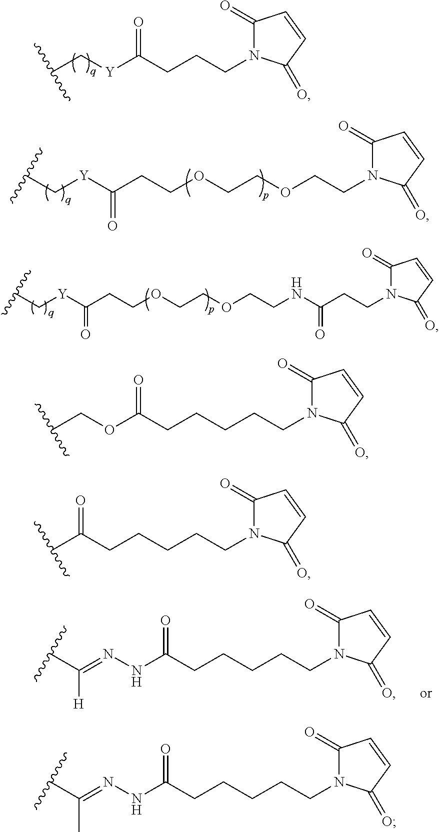

wherein Y is O or NH; q is 0, 1, 2 or 3; and p is 0, 1, 2 or 3.

In some embodiments, the compound of Formula B has the following structure:

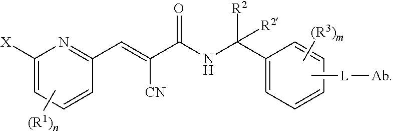

### Pharmaceutical Compositions

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that are used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, the compounds described herein are administered either alone or in combination with pharmaceutically acceptable carriers, excipients or diluents, in a pharmaceutical composition. Administration of the compounds and compositions described herein can be effected by any method that enables delivery of the compounds to the site of action. These methods include, though are not limited to delivery via enteral routes (including oral, gastric or duodenal feeding tube, rectal suppository and rectal enema), parenteral routes (injection or infusion, including intraarterial, intracardiac, intradermal, intraduodenal, intramedullary, intramuscular, intraosseous, intraperitoneal, intrathecal, intravascular, intravenous, intravitreal, epidural and subcutaneous), inhalational, transdermal, transmucosal, sublingual, buccal and topical (including epicutaneous, dermal, enema, eye drops, ear drops, intranasal, vaginal) administration, although the most suitable route may depend upon for example the condition and disorder of the recipient. By way of example only, compounds described herein can be administered locally to the area in need of treatment, by for example, local infusion during surgery, topical application such as creams or ointments, injection, catheter, or implant. The administration can also be by direct injection at the site of a diseased tissue or organ.

In some embodiments, pharmaceutical compositions suitable for oral administration are presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. In some embodiments, the active ingredient is presented as a bolus, electuary or paste.

Pharmaceutical compositions which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. In some embodiments, the tablets are coated or scored and are formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In some embodiments, stabilizers are added.

In some embodiments, pharmaceutical compositions are formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The compositions may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Pharmaceutical compositions for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical compositions may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example, as an emulsion in an acceptable oil) or ion exchange resins, or as sparingly soluble derivatives, for example, as a sparingly soluble salt.

Pharmaceutical compositions for administration by inhalation are conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, pharmaceutical preparations may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

It should be understood that in addition to the ingredients particularly mentioned above, the compounds and compositions described herein may include other agents conventional in the art having regard to the type of formulation in question, for example those suitable for oral administration may include flavoring agents.

### Methods of Dosing and Treatment Regimens

In one embodiment, the compounds described herein, or a pharmaceutically acceptable salt thereof, are used in the preparation of medicaments for the treatment of diseases or conditions in a mammal that would benefit from inhibition or reduction of the activity of one or more deubiquitinase enzymes. Methods for treating any of the diseases or conditions described herein in a mammal in need of such treatment, involves administration of pharmaceutical compositions that include at least one compound described herein or a pharmaceutically acceptable salt, active metabolite, prodrug, or pharmaceutically acceptable solvate thereof, in therapeutically effective amounts to said mammal.

In certain embodiments, the compositions containing the compound(s) described herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. When used in patients, effective amounts for this use will depend on the severity and course of the disease, disorder or condition, previous therapy, the patient's health status and response to the drugs, and the judgment of the treating physician. In one aspect, prophylactic treatments include administering to a mammal, which previously experienced at least one symptom of the disease being treated and is currently in remission, a pharmaceutical composition comprising a compound described herein, or a pharmaceutically acceptable salt thereof, in order to prevent a return of the symptoms of the disease or condition.

In certain embodiments wherein the patient's condition does not improve, upon the doctor's discretion the administration of the compounds are administered chronically, that is, for an extended period of time, including throughout the duration of the patient's life in order to ameliorate or otherwise control or limit the symptoms of the patient's disease or condition.

In certain embodiments wherein a patient's status does improve, the dose of drug being administered is temporarily reduced or temporarily suspended for a certain length of time (i.e., a “drug holiday”). In specific embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

Once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the patient requires intermittent treatment on a long-term basis upon any recurrence of symptoms.

The amount of a given agent that corresponds to such an amount varies depending upon factors such as the particular compound, disease condition and its severity, the identity (e.g., weight, sex) of the subject or host in need of treatment, but nevertheless is determined according to the particular circumstances surrounding the case, including, e.g., the specific agent being administered, the route of administration, the condition being treated, and the subject or host being treated.

In general, however, doses employed for adult human treatment are typically in the range of 0.01 mg-5000 mg per day. In one aspect, doses employed for adult human treatment are from about 1 mg to about 1000 mg per day. In one embodiment, the desired dose is conveniently presented in a single dose or in divided doses administered simultaneously or at appropriate intervals, for example as two, three, four or more sub-doses per day.

In one embodiment, the daily dosages appropriate for the compound described herein, or a pharmaceutically acceptable salt thereof, are from about 0.01 to about 50 mg/kg per body weight. In some embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens are determined by standard pharmaceutical procedures in cell cultures or experimental animals, including, but not limited to, the determination of the LD₅₀ and the ED₅₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the compounds described herein lies within a range of circulating concentrations that include the ED₅₀ with minimal toxicity. In certain embodiments, the daily dosage range and/or the unit dosage amount varies within this range depending upon the dosage form employed and the route of administration utilized.

In any of the aforementioned aspects are further embodiments in which the effective amount of the compound described herein, or a pharmaceutically acceptable salt thereof, is: (a) systemically administered to the mammal; and/or (b) administered orally to the mammal; and/or (c) intravenously administered to the mammal; and/or (d) administered by injection to the mammal; and/or (e) administered topically to the mammal; and/or (f) administered non-systemically or locally to the mammal.

In any of the aforementioned aspects are further embodiments comprising single administrations of the effective amount of the compound, including further embodiments in which (i) the compound is administered once a day; or (ii) the compound is administered to the mammal multiple times over the span of one day.

In any of the aforementioned aspects are further embodiments comprising multiple administrations of the effective amount of the compound, including further embodiments in which (i) the compound is administered continuously or intermittently: as in a single dose; (ii) the time between multiple administrations is every 6 hours; (iii) the compound is administered to the mammal every 8 hours; (iv) the compound is administered to the mammal every 12 hours; (v) the compound is administered to the mammal every 24 hours. In further or alternative embodiments, the method comprises a drug holiday, wherein the administration of the compound is temporarily suspended or the dose of the compound being administered is temporarily reduced; at the end of the drug holiday, dosing of the compound is resumed. In one embodiment, the length of the drug holiday varies from 2 days to 1 year.

In certain instances, it is appropriate to administer at least one compound described herein, or a pharmaceutically acceptable salt thereof, in combination with one or more other therapeutic agents. In certain embodiments, the pharmaceutical composition further comprises one or more anti-cancer agents. In further or alternative embodiments, the pharmaceutical composition further comprises one or more anti-cancer agents bound to human serum albumin e.g. Abraxane.

In one embodiment, the therapeutic effectiveness of one of the compounds described herein is enhanced by administration of an adjuvant (i.e., by itself the adjuvant has minimal therapeutic benefit, but in combination with another therapeutic agent, the overall therapeutic benefit to the patient is enhanced). Or, in some embodiments, the benefit experienced by a patient is increased by administering one of the compounds described herein with another agent (which also includes a therapeutic regimen) that also has therapeutic benefit.

In one specific embodiment, a compound described herein, or a pharmaceutically acceptable salt thereof, is co-administered with a second therapeutic agent, wherein the compound described herein, or a pharmaceutically acceptable salt thereof, and the second therapeutic agent modulate different aspects of the disease, disorder or condition being treated, thereby providing a greater overall benefit than administration of either therapeutic agent alone.

In any case, regardless of the disease, disorder or condition being treated, the overall benefit experienced by the patient is simply be additive of the two therapeutic agents or the patient experiences a synergistic benefit.

In certain embodiments, different therapeutically-effective dosages of the compounds disclosed herein will be utilized in formulating pharmaceutical composition and/or in treatment regimens when the compounds disclosed herein are administered in combination with one or more additional agent, such as an additional therapeutically effective drug, an adjuvant or the like. Therapeutically-effective dosages of drugs and other agents for use in combination treatment regimens is optionally determined by means similar to those set forth hereinabove for the actives themselves. Furthermore, the methods of prevention/treatment described herein encompasses the use of metronomic dosing, i.e., providing more frequent, lower doses in order to minimize toxic side effects. In some embodiments, a combination treatment regimen encompasses treatment regimens in which administration of a compound described herein, or a pharmaceutically acceptable salt thereof, is initiated prior to, during, or after treatment with a second agent described herein, and continues until any time during treatment with the second agent or after termination of treatment with the second agent. It also includes treatments in which a compound described herein, or a pharmaceutically acceptable salt thereof, and the second agent being used in combination are administered simultaneously or at different times and/or at decreasing or increasing intervals during the treatment period. Combination treatment further includes periodic treatments that start and stop at various times to assist with the clinical management of the patient.

It is understood that the dosage regimen to treat, prevent, or ameliorate the condition(s) for which relief is sought, is modified in accordance with a variety of factors (e.g. the disease, disorder or condition from which the subject suffers; the age, weight, sex, diet, and medical condition of the subject). Thus, in some instances, the dosage regimen actually employed varies and, in some embodiments, deviates from the dosage regimens set forth herein.

For combination therapies described herein, dosages of the co-administered compounds vary depending on the type of co-drug employed, on the specific drug employed, on the disease or condition being treated and so forth. In additional embodiments, when co-administered with one or more other therapeutic agents, the compound provided herein is administered either simultaneously with the one or more other therapeutic agents, or sequentially.

In combination therapies, the multiple therapeutic agents (one of which is one of the compounds described herein) are administered in any order or even simultaneously. If administration is simultaneous, the multiple therapeutic agents are, by way of example only, provided in a single, unified form, or in multiple forms (e.g., as a single pill or as two separate pills).

The compounds described herein, or a pharmaceutically acceptable salt thereof, as well as combination therapies, are administered before, during or after the occurrence of a disease or condition, and the timing of administering the composition containing a compound varies. Thus, in one embodiment, the compounds described herein are used as a prophylactic and are administered continuously to subjects with a propensity to develop conditions or diseases in order to prevent the occurrence of the disease or condition. In another embodiment, the compounds and compositions are administered to a subject during or as soon as possible after the onset of the symptoms. In specific embodiments, a compound described herein is administered as soon as is practicable after the onset of a disease or condition is detected or suspected, and for a length of time necessary for the treatment of the disease. In some embodiments, the length required for treatment varies, and the treatment length is adjusted to suit the specific needs of each subject. For example, in specific embodiments, a compound described herein or a formulation containing the compound is administered for at least 2 weeks, about 1 month to about 5 years.

In some embodiments, a compound described herein, or a pharmaceutically acceptable salt thereof, is administered in combination with chemotherapy, hormone blocking therapy, radiation therapy, monoclonal antibodies, or combinations thereof.

Chemotherapy includes the use of anti-cancer agents.

### EXAMPLES

The following examples are provided for illustrative purposes only and not to limit the scope of the claims provided herein.

### Example 1: HA-Ubiqutin-Vinylsulfone (HA-Ub-VS) Cell Labeling

The ability of test compounds to inhibit DUB activity is measured in cells (NCI-H929, Z138, MM.1S, A375) using a hemaglutinin-ubiquitin-vinylsulfone (HA-Ub-VS) labeling assay. Briefly, 1-2×10⁶ cells are plated in 12-well plates in 1 ml complete growth media and 250 μl of test compound in 0.5% dimethylsulfoxide (or DMSO control) is added to each well. Plates are incubated at 37° C., 5% CO₂ for 2-6 hours before cells are harvested into eppendorf tubes, washed once in PBS and resuspended in 100 μl cold lysis buffer (50 mM Tris pH 8, 150 mM NaCl, 0.5% NP-40, 2 mM phenylmethylsulfonyl fluoride and complete protease inhibitor tablet). Resupended cells are frozen on dry ice, then thawed and incubated on ice for 15 minutes. Tubes are centrifuged for 10 minutes at 14,000 rpm at 4° C. and the cleared supernatants transferred to clean tubes. To assay for inhibition of DUB activity, 20-45 μl of the cleared supernatant is removed to a clean tube containing 2-5 μl of 2.5 μM HA-Ubiquitin-Vinylsulfone and mixed. The samples are incubated 15-60 min at 37° C. before the addition of SDS gel loading buffer. Proteins are separated by SDS-PAGE, transferred to nitrocellulose and the blot probed with anti-HA antibody and anti-β-actin antibodies. The HA and β-actin antibodies are detected using secondary antibodies coupled to IRdye 800 or IRdye 680 and visualized and quantitated using the LiCor Odyssey machine.

### Example 2: Analysis of Mcl-1 Expression and PARP Cleavage

The ability of test compounds to decrease expression of the tumor promoting protein, Mcl-1, and to induce the inactivation/cleavage of the PARP protein is evaluated in cells (NCI-H929, Z138, MM.1S) by Western blotting. Cells are plated and treated as described in the HA-Ub-VS cell labeling assay (see above). A portion of the cleared lysate (15-50 μl) is run on an SDS-polyacrylamide gel to separate proteins and then transferred to nitrocellulose. After transfer to nitrocellulose, the proteins are detected by probing the blot with anti-Mcl-1 and anti-PARP antibodies followed by secondary antibodies coupled to IRdye 800 and IRdye 680 and visualization and quantitation using a LiCor Odyssey machine. Activity of the test compound is identified as a significant decrease in Mcl-1 expression relative to the vehicle treated cells and to the appearance of a cleaved fragment of PARD relative to the vehicle-treated cells.

### Example 3: In Vivo Tumor Growth

A xenograft model is used to test efficacy of compounds in inhibiting tumor growth. Briefly, 1×10⁶-2×10⁷ tumor cell suspensions (Z138, A375, MM.1S) are injected subcutaneously into the flank regions of mice (SCID, NSG, nude) in an equal volume of matrigel. Tumor volumes are measured using calipers and when the tumor volumes are ˜100 mm³, test compound is administered every 1-3 days by oral gavage or by injection (intraperitoneal, intravenous, subcutaneous) until tumor volumes of the vehicle treated animals are ˜1000-2000 mm³. Inhibition of primary tumor growth is evident as a significant decrease in tumor volume relative to the vehicle treated mice.

### Example 4: (E)-3-(6-Bromopyridin-2-yl)-2-cyano-N—S-(1-phenylbutyl)acrylamide (WP1130)

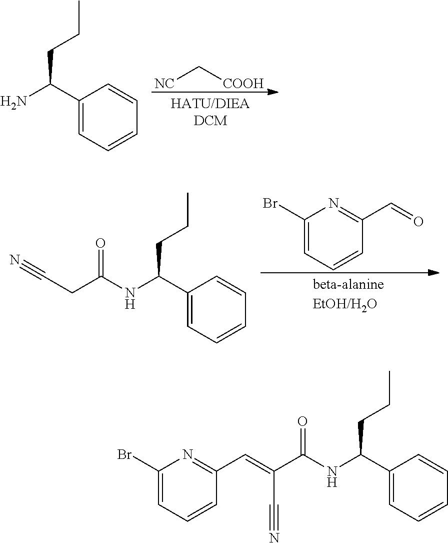

To a mixture of cyanoacetic acid (1.0 g, 11.8 mmol) and 1-[bis(dimethylamino)methylene]-1H-1,2,3-triazolo[4,5-b]pyridinium 3-oxid hexafluorophosphate (HATU) (4.48 g, 11.8 mmol) in dichloromethane (5 mL) was added 1-phenylcyclopentan-1-amine (81 mg, 0.5 mmol), followed by N,N-diisopropylethylamine (4.1 mL, 23.6 mmol). The mixture was stirred at room temperature for overnight then evaporated to dryness. The residue was extracted with EtOAc and water. The organic layer was separated, washed with brine, dried over Na₂SO₄ and concentrated to dryness. The residue was re-dissolved in a mixture of EtOH (2 mL) and water (2 mL). To the mixture was then added β-alanine (8.4 g, 94.4 mmol), followed by 6-bromo-2-pyridinecarboxaldehyde (4.4 g 23.6 mmol). The resulting mixture was then stirred at room temperature overnight and a white precipitate formed. The product was then collected via filtration, and rinsed with a solution of 1:1 EtOH:water 3× to afford WP1130 as a white solid (3.2 g). LCMS MH+=384

### Example 5: (E)-3-(6-Bromopyridin-2-yl)-2-cyano-N—S-(1-phenylbutyl)acrylamide (WP1130)-Human Serum Albumin (HSA) Adduct

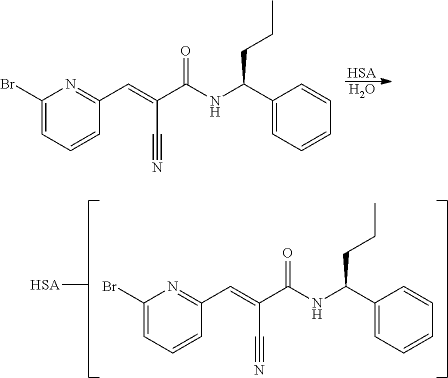

Human serum albumin (HSA) (665 mg, 0.1 mmol) loaded into a 40 mL scintillation vial and then distilled water was added (3.0 mL). The mixture was heated at 37° C. until all of the HSA was dissolved (clear, yellow/beige solution). To the mixture was added WP1130 (7.68 mg, 0.2 mmol) in acetonitrile (0.2 mL). The mixture was sonicated for 10 mins and heated at 37° C. in a rotary shaker bath. The heating was continued until WP1130 was no longer observed by LCMS (48 hrs). The water and volatiles were evaporated and the resulting powder was dried under vacuum for 24 hrs. The resulting powder was pressed using a mortar and pestle to give a pale yellow powder.

### Example 6: Release of the WP1130 from HSA as methyl 2-((1-(6-bromopyridin-2-yl)-2-cyano-3-oxo-3-(((S)-1-phenylbutyl)amino)propyl)thio)acetate

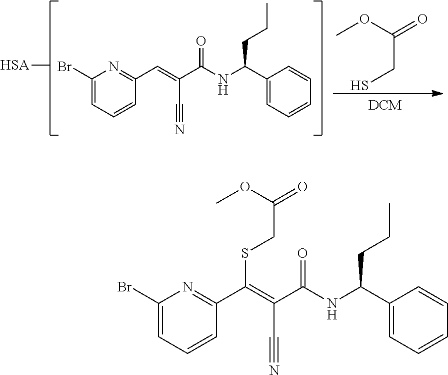

The product from Example 5 (100 mg,) loaded into a 4.0 mL scintillation vial and distilled water was added (0.30 mL). The mixture was heated at 37° C. until all of the HSA.WP1130 adduct was dissolved (clear, yellow/beige solution). To the mixture was added methyl mercaptoacetoaceate (1.25 μl). The target product methyl 2-((1-(6-bromopyridin-2-yl)-2-cyano-3-oxo-3-(((S)-1-phenylbutyl)amino)propyl)thio)acetate (the WP1130-methyl mercaptoacetoaceate adduct) was detected by LCMS M+Na=512

The examples and embodiments described herein are for illustrative purposes only and various modifications or changes suggested to persons skilled in the art are to be included within the spirit and purview of this application and scope of the appended claims.

What is claimed is:

1. A method of treating a disease or condition in a mammal that would benefit from the inhibition of the activity of a deubiquitinating enzyme comprising administering to the mammal a pharmaceutical composition comprising a deubiquitinase inhibitor and albumin.

2. The method of claim 1, wherein the disease or condition is cancer, fibrosis, an autoimmune disease or condition, an inflammatory disease or condition, a neurodegenerative disease or condition or an infection.

3. A method of enhancing transport of a deubiquitinase inhibitor to a tumor cell or inflammed tissue in a mammal, or enhancing binding of a deubiquitinase inhibitor to a tumor cell in a mammal, or increasing the accumulation of a deubiquitinase inhibitor in a tumor or inflamed tissue of a mammal, or improving the pharmacokinetic profile of a deubiquitinase inhibitor in a mammal, comprising administering to the mammal a pharmaceutical composition comprising a deubiquitinase inhibitor and albumin.

4. The method of any one of claims 1 to 3, wherein the pharmaceutical composition is administered to the mammal intravenously or subcutaneously.

5. The method of any one of claims 1 to 4, wherein the albumin is human serum albumin and the deubiquitinase inhibitor is a small molecule cyano-substituted acrylamide compound.

6. The method of any one of claims 1 to 5, wherein the deubiquitinase inhibitor has the following structure: 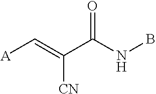
wherein,
A is a substituted or unsubstituted heteroaryl or substituted or unsubstituted aryl; and
B is a substituted or unsubstituted -(alkylene)-aryl or a substituted or unsubstituted -(alkylene)-heteroaryl.

7. The method of any one of claims 1 to 6, wherein the deubiquitinase inhibitor has the structure of Formula (I), or a pharmaceutically acceptable salt, or solvate thereof: 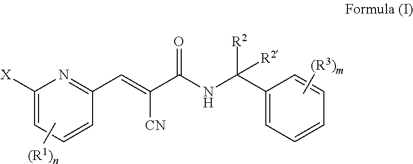
wherein,
X is H, F, Cl, Br, —OH, alkyl, —O-alkyl, —S-alkyl, —NH₂ or —NH-alkyl;
R¹ is H, halogen, CN, NO₂, unsubstituted or substituted alkyl, unsubstituted or substituted cycloalkyl, —O-(unsubstituted or substituted alkyl), —S-(unsubstituted or substituted alkyl), or —NH-(unsubstituted or substituted alkyl);
or R¹ and X are taken together with the intervening atoms connecting R¹ and X to form a heterocyclic ring with 1 or 2 heteroatoms selected from O, N, and S;
R² is H, unsubstituted or substituted alkyl, unsubstituted or substituted heteroalkyl, unsubstituted or substituted cycloalkyl, unsubstituted or substituted heterocycloalkyl, or unsubstituted or substituted benzyl;
or R² is -L¹-R⁶ where,
L¹ is bond, unsubstituted or substituted alkyl, unsubstituted or substituted cycloalkyl, or unsubstituted or substituted heteroalkyl;
R⁶ is unsubstituted or substituted cycloalkyl, unsubstituted or substituted heterocycloalkyl, unsubstituted or substituted aryl, or unsubstituted or substituted heteroaryl;
R^{2′} is H or unsubstituted or substituted alkyl;
each R³ is independently selected from H, halogen, —CN, —NO₂, —OH, —OR⁴, —SR⁴, —S(═O)R⁴, —S(═O)₂R⁴, —S(═O)₂N(R⁵)₂, —NR⁵S(═O)₂R⁴, —C(═O)R⁴, —OC(═O)R⁴, —CO₂R⁵, —OCO₂R⁴, —N(R⁵)₂, —C(═O)N(R⁵)₂, —OC(═O)N(R⁵)₂, —NHC(═O)R⁴, —NHC(═O)OR⁴, unsubstituted or substituted alkyl, unsubstituted or substituted alkenyl, unsubstituted or substituted alkynyl, unsubstituted or substituted heteroalkyl, unsubstituted or substituted cycloalkyl, unsubstituted or substituted aryl, unsubstituted or substituted heteroaryl, and -L²-L³-L⁴-L⁵-R⁷;
L² is absent, —O—, —S—, —S(O)—, —S(O)₂₋, —NR⁴—, —CH(OH)—, —C(═O)—, —C(═O)NH—, —NHC(═O)—, —C(═O)O—, —OC(═O)—, —CH(═N)—, —CH(═N—NH)—, —CCH₃(═N)—, —CCH₃(═N—NH)—, —OC(═O)NH—, —NHC(═O)NH—, —NHC(═O)O—, —(CH₂)ₚ—, or —(OCH₂CH₂)ₚ—, p is 1, 2, 3, or 4;
L³ is absent, unsubstituted or substituted alkylene, unsubstituted or substituted heteroalkylene, unsubstituted or substituted alkenylene, unsubstituted or substituted alkynylene, unsubstituted or substituted cycloalkylene, unsubstituted or substituted heterocycloalkylene, unsubstituted or substituted arylene, unsubstituted or substituted heteroarylene, or —(OCH₂CH₂)ₚ—, p is 1, 2, 3, or 4;
L⁴ is absent, —O—, —S—, —S(O)—, —S(O)₂₋, —NR⁴—, —CH(OH)—, —C(═O)—, —C(═O)NH—, —NHC(═O)—, —C(═O)O—, —OC(═O)—, —OC(═O)NH—, —NHC(═O)NH—, or —NHC(═O)O—;
L⁵ is absent, unsubstituted or substituted alkylene, unsubstituted or substituted heteroalkylene;
R⁷ is H, halogen, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, or unsubstituted or substituted heteroaryl;
R⁴ is C₁-C₆alkyl, C₁-C₆fluoroalkyl, C₁-C₆deuteroalkyl, C₃-C₆cycloalkyl, a substituted or unsubstituted phenyl, a substituted or unsubstituted monocyclic heteroaryl, or a substituted or unsubstituted bicyclic heteroaryl;
each R⁵ is independently selected from H, C₁-C₆alkyl, C₁-C₆fluoroalkyl, C₁-C₆deuteroalkyl, C₃-C₆cycloalkyl, a substituted or unsubstituted phenyl, or a substituted or unsubstituted monocyclic heteroaryl; or
two R⁵ groups attached to the same N atom are taken together with the N atom to which they are attached to form a substituted or unsubstituted heterocycle
m is 0, 1, 2, 3, or 4;
n is 0, 1, 2 or 3.

8. The method of any one of claims 1 to 7, wherein the deubiquitinase inhibitor is physically bound to the albumin through non-covalent interactions.

9. The method of any one of claims 1 to 8, wherein the deubiquitinase inhibitor and albumin are formulated as particles; and wherein particles of the deubiquitinase inhibitor are coated with the albumin.

10. The method of any one of claims 1 to 9, wherein the ratio of the deubiquitinase inhibitor to albumin is from about 1:20 to about 20:1.

11. The method of any one of claims 1 to 5, wherein the deubiquitinase inhibitor is covalently conjugated with an albumin binding group with or without a linker as depicted with the following Formula B:
(DUB)-L-Ab Formula B
wherein,
DUB is a deubiquitinase inhibitor;
L is absent or a linker;
Ab is an albumin binding group.

12. The method of claim 11, wherein the deubiquitinase inhibitor has the following structure: 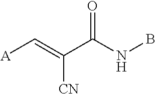
wherein,
A is a substituted or unsubstituted heteroaryl or substituted or unsubstituted aryl; and
B is a substituted or unsubstituted -(alkylene)-aryl or a substituted or unsubstituted -(alkylene)-heteroaryl.

13. The method of claim 11 or claim 12, wherein the albumin binding group increases the binding affinity of the deubiquitinase inhibitor to human serum albumin.

14. The method of claim 13, wherein the albumin binding group is a fatty acid, steroid, or thyroid hormone.

15. The method of any one of claims 11 to 14, wherein the albumin binding group is a saturated alkyl acid or an unsaturated alkyl acid that is a butanoic acid (butyric acid), pentanoic acid (valeric acid), hexanoic acid (caproic acid), heptanoic acid (enanthic acid), octanoic acid (caprylic acid), nonanoic acid (pelargonic acid), decanoic acid (capric acid), undecanoic acid (undecylic acid), dodecanoic acid (lauric acid), tridecanoic acid (tridecylic acid), tetradecanoic acid (myristic acid), pentadecanoic acid (pentadecylic acid), hexadecanoic acid (palmitic acid), heptadecanoic acid (margaric acid), octadecanoic acid (stearic acid), nonadecanoic acid (nonadecylic acid), eicosanoic acid (arachidic acid), heneicosanoic acid, docosanoic acid (behenic acid), tricosanoic acid, tetracosanoic acid (lignoceric acid), pentacosanoic acid, hexaconsanoic acid (cerotic acid), heptacosanoic acid, octacosanoic acid (montanic acid), nonacosanoic acid, triacontanoic acid (melissic acid), or hentriacontanoic acid.

16. The method of any one of claims 11 to 15, wherein the albumin binding group is myristic acid.

17. The method of claim 11, wherein the albumin binding group is a saturated alkyl acid that is covalently conjugated to the deubiquitinase inhibitor as depicted with the following Formula B-1:
(DUB)-L-X—Y-(Alk) Formula B-1
wherein,
DUB is a deubiquitinase inhibitor;
L is absent or a linker;
X is absent, —O—, —S— or —NH—;
Y is —C(═O)—;
Alk is C₆-C₄₀alkyl.

18. The method of any one of claims 11 to 17, wherein L is —CH═N—, —C(alkyl)=N—, —CH═N—NH—, —C(alkyl)=N—NH—, or —S—S—.

19. The method of claim 17, wherein Alk is —(CH₂)₅—CH₃, —(CH₂)₆—CH₃, —(CH₂)₇—CH₃, —(CH₂)₈—CH₃, —(CH₂)₉—CH₃, —(CH₂)₁₀—CH₃, —(CH₂)₁₁—CH₃, —(CH₂)₁₂—CH₃, —(CH₂)₁₃—CH₃, —(CH₂)₁₄—CH₃, —(CH₂)₁₅—CH₃, —(CH₂)₁₆—CH₃, —(CH₂)₁₇—CH₃, —(CH₂)₁₈—CH₃, —(CH₂)₁₉—CH₃, —(CH₂)₂₀—CH₃, —(CH₂)₂₁—CH₃, —(CH₂)₂₂—CH₃, —(CH₂)₂₃—CH₃, —(CH₂)₂₄—CH₃, —(CH₂)₂₅—CH₃, —(CH₂)₂₆—CH₃, —(CH₂)₂₇—CH₃, —(CH₂)₂₈—CH₃, —(CH₂)₂₉—CH₃, —(CH₂)₃₀—CH₃, —(CH₂)₃₁—CH₃, —(CH₂)₃₂—CH₃, —(CH₂)₃₃—CH₃, —(CH₂)₃₄—CH₃, —(CH₂)₃₅—CH₃, —(CH₂)₃₆—CH₃, —(CH₂)₃₇—CH₃, —(CH₂)₃₈—CH₃, or —(CH₂)₃₉—CH₃.

20. The method of claim 19, wherein Alk is —(CH₂)₁₂—CH₃.

21. The method of claim 11 or claim 12, wherein the albumin binding group is capable of covalently binding to the free thiol (Cys 34), the free —NH₂ (lysine(s)) or the guanidine group of arginine(s) of human serum albumin.

22. The method of claim 21, wherein the albumin binding group comprises a maleimide group, a halogenacetamide group, a halogenacetyl group, a halogenacetate group, a pyridinylthio group, a vinylcarbonyl group, an aziridinyl group, a disulfide group, an acetylene group, a hydroxysuccinimide group or a thiol group.

23. The method of claim 21 or claim 22, wherein the albumin binding group comprises a maleimide group:

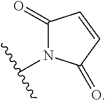 [SMILES: *N1C(=O)C=CC1=O]

24. The method of claim 23, wherein L is absent, 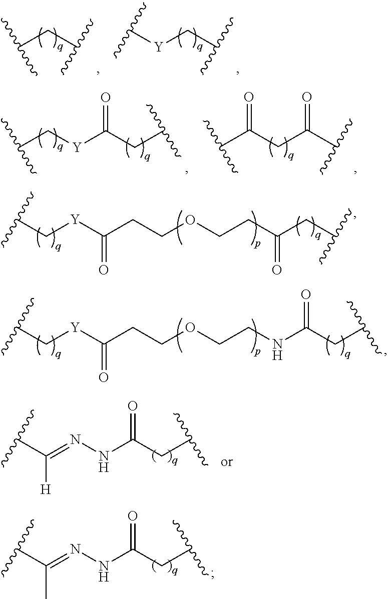
wherein Y is O or NH; each q is independently selected from 0, 1, 2, 3, 4, and 5; and p is 0, 1, 2, 3 or 4.

25. The method of claim 23, wherein -L-Ab is: 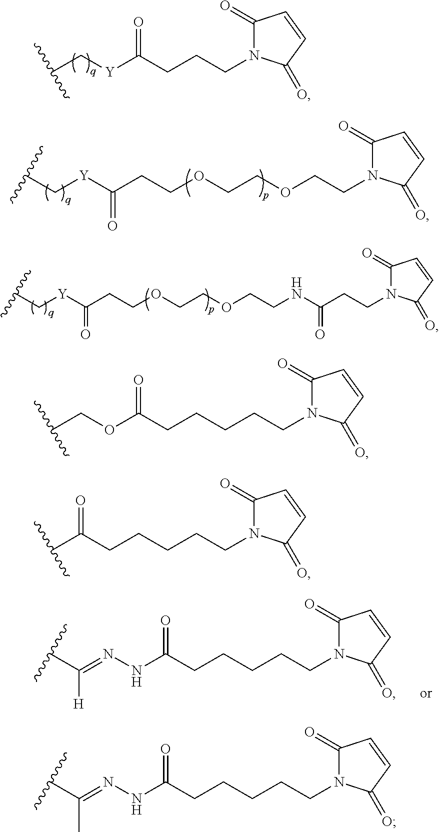
wherein Y is O or NH; q is 0, 1, 2 or 3; and p is 0, 1, 2 or 3.

26. A pharmaceutical composition comprising a deubiquitinase inhibitor and albumin.

27. The pharmaceutical composition of claim 26, wherein the albumin is human serum albumin.

28. The pharmaceutical composition of claim 26 or claim 27, wherein the ratio of the deubiquitinase inhibitor to albumin is from about 1:20 to about 20:1.

29. The pharmaceutical composition of any one of claims 26 to 28, wherein the pharmaceutical composition is formulated for intravenous or subcutaneous injection to a mammal.

30. The pharmaceutical composition of any one of claims 26 to 29, wherein the deubiquitinase inhibitor has the following structure: 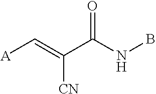
wherein,
A is a substituted or unsubstituted heteroaryl or substituted or unsubstituted aryl; and
B is a substituted or unsubstituted -(alkylene)-aryl or a substituted or unsubstituted -(alkylene)-heteroaryl.

31. The pharmaceutical composition of claim 30, wherein the deubiquitinase inhibitor has the structure of Formula (I), or a pharmaceutically acceptable salt, or solvate thereof: 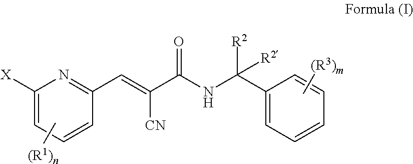
wherein,
X is H, F, Cl, Br, —OH, alkyl, —O-alkyl, —S-alkyl, —NH₂ or —NH-alkyl;
R¹ is H, halogen, CN, NO₂, unsubstituted or substituted alkyl, unsubstituted or substituted cycloalkyl, —O-(unsubstituted or substituted alkyl), —S-(unsubstituted or substituted alkyl), or —NH-(unsubstituted or substituted alkyl);
or R¹ and X are taken together with the intervening atoms connecting R¹ and X to form a heterocyclic ring with 1 or 2 heteroatoms selected from O, N, and S;
R² is H, unsubstituted or substituted alkyl, unsubstituted or substituted heteroalkyl, unsubstituted or substituted cycloalkyl, unsubstituted or substituted heterocycloalkyl, or unsubstituted or substituted benzyl;
or R² is -L¹-R⁶ where,
L¹ is bond, unsubstituted or substituted alkyl, unsubstituted or substituted cycloalkyl, or unsubstituted or substituted heteroalkyl;
R⁶ is unsubstituted or substituted cycloalkyl, unsubstituted or substituted heterocycloalkyl, unsubstituted or substituted aryl, or unsubstituted or substituted heteroaryl;
R^{2′} is H or unsubstituted or substituted alkyl;
each R³ is independently selected from H, halogen, —CN, —NO₂, —OH, —OR⁴, —SR⁴, —S(═O)R⁴, —S(═O)₂R⁴, —S(═O)₂N(R⁵)₂, —NR⁵S(═O)₂R⁴, —C(═O)R⁴, —OC(═O)R⁴, —CO₂R⁵, —OCO₂R⁴, —N(R⁵)₂, —C(═O)N(R⁵)₂, —OC(═O)N(R⁵)₂, —NHC(═O)R⁴, —NHC(═O)OR⁴, unsubstituted or substituted alkyl, unsubstituted or substituted alkenyl, unsubstituted or substituted alkynyl, unsubstituted or substituted heteroalkyl, unsubstituted or substituted cycloalkyl, unsubstituted or substituted aryl, unsubstituted or substituted heteroaryl, and -L²-L³-L⁴-L⁵-R⁷;
L² is absent, —O—, —S—, —S(O)—, —S(O)₂₋, —NR⁴—, —CH(OH)—, —C(═O)—, —C(═O)NH—, —NHC(═O)—, —C(═O)O—, —OC(═O)—, —CH(═N)—, —CH(═N—NH)—, —CCH₃(═N)—, —CCH₃(═N—NH)—, —OC(═O)NH—, —NHC(═O)NH—, —NHC(═O)O—, —(CH₂)ₚ—, or —(OCH₂CH₂)ₚ—, p is 1, 2, 3, or 4;
L³ is absent, unsubstituted or substituted alkylene, unsubstituted or substituted heteroalkylene, unsubstituted or substituted alkenylene, unsubstituted or substituted alkynylene, unsubstituted or substituted cycloalkylene, unsubstituted or substituted heterocycloalkylene, unsubstituted or substituted arylene, unsubstituted or substituted heteroarylene, or —(OCH₂CH₂)ₚ—, p is 1, 2, 3, or 4;
L⁴ is absent, —O—, —S—, —S(O)—, —S(O)₂₋, —NR⁴—, —CH(OH)—, —C(═O)—, —C(═O)NH—, —NHC(═O)—, —C(═O)O—, —OC(═O)—, —OC(═O)NH—, —NHC(═O)NH—, or —NHC(═O)O—;
L⁵ is absent, unsubstituted or substituted alkylene, unsubstituted or substituted heteroalkylene;
R⁷ is H, halogen, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, or unsubstituted or substituted heteroaryl;
R⁴ is C₁-C₆alkyl, C₁-C₆fluoroalkyl, C₁-C₆deuteroalkyl, C₃-C₆cycloalkyl, a substituted or unsubstituted phenyl, a substituted or unsubstituted monocyclic heteroaryl, or a substituted or unsubstituted bicyclic heteroaryl;
each R⁵ is independently selected from H, C₁-C₆alkyl, C₁-C₆fluoroalkyl, C₁-C₆deuteroalkyl, C₃-C₆cycloalkyl, a substituted or unsubstituted phenyl, or a substituted or unsubstituted monocyclic heteroaryl; or
two R⁵ groups attached to the same N atom are taken together with the N atom to which they are attached to form a substituted or unsubstituted heterocycle
m is 0, 1, 2, 3, or 4;
n is 0, 1, 2, or 3.

32. The pharmaceutical composition of any one of claims 26 to 31, wherein the deubiquitinase inhibitor is physically bound to the albumin through non-covalent interactions.

33. The pharmaceutical composition of claim 32, comprising albumin coated particles of the deubiquitinase inhibitor.

34. The pharmaceutical composition of any one of claims 26 to 30, wherein the deubiquitinase inhibitor is covalently conjugated with an albumin binding group with or without a linker as depicted with the following Formula B:
(DUB)-L-Ab Formula B
wherein,
DUB is a deubiquitinase inhibitor;
L is a absent or a linker;
Ab is an albumin binding group.

35. The pharmaceutical composition of claim 34, wherein the deubiquitinase inhibitor has the following structure: 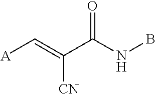
wherein,
A is a substituted or unsubstituted heteroaryl or substituted or unsubstituted aryl; and
B is a substituted or unsubstituted -(alkylene)-aryl or a substituted or unsubstituted -(alkylene)-heteroaryl.

36. The pharmaceutical composition of claim 34 or claim 35, wherein the albumin binding group increases the binding affinity of the deubiquitinase inhibitor to human serum albumin.

37. The pharmaceutical composition of any one of claims 34 to 36, wherein the albumin binding group is a fatty acid, steroid, or thyroid hormone.

38. The pharmaceutical composition of any one of claims 34 to 37, wherein the albumin binding group is a saturated alkyl acid or an unsaturated alkyl acid that is a butanoic acid (butyric acid), pentanoic acid (valeric acid), hexanoic acid (caproic acid), heptanoic acid (enanthic acid), octanoic acid (caprylic acid), nonanoic acid (pelargonic acid), decanoic acid (capric acid), undecanoic acid (undecylic acid), dodecanoic acid (lauric acid), tridecanoic acid (tridecylic acid), tetradecanoic acid (myristic acid), pentadecanoic acid (pentadecylic acid), hexadecanoic acid (palmitic acid), heptadecanoic acid (margaric acid), octadecanoic acid (stearic acid), nonadecanoic acid (nonadecylic acid), eicosanoic acid (arachidic acid), heneicosanoic acid, docosanoic acid (behenic acid), tricosanoic acid, tetracosanoic acid (lignoceric acid), pentacosanoic acid, hexaconsanoic acid (cerotic acid), heptacosanoic acid, octacosanoic acid (montanic acid), nonacosanoic acid, triacontanoic acid (melissic acid), or hentriacontanoic acid.

39. The pharmaceutical composition of any one of claims 34 to 38, wherein the albumin binding group is myristic acid.

40. The pharmaceutical composition of any one of claims 34 to 36, wherein the albumin binding group is a saturated alkyl acid that is covalently conjugated to the deubiquitinase inhibitor as depicted with the following Formula B-1:
(DUB)-L-X—Y-(Alk) Formula B-1
wherein,
DUB is a deubiquitinase inhibitor described herein;
L is a absent or a linker;
X is absent, —O—, —S— or —NH—;
Y is —C(═O)—;
Alk is C₆-C₄₀alkyl.

41. The pharmaceutical composition of any one of claims 34 to 40, wherein L is —CH═N—, —C(alkyl)=N—, —CH═N—NH—, —C(alkyl)=N—NH—, or —S—S—.

42. The pharmaceutical composition of claim 40, wherein Alk is —(CH₂)₅—CH₃, —(CH₂)₆—CH₃, —(CH₂)₇—CH₃, —(CH₂)₈—CH₃, —(CH₂)₉—CH₃, —(CH₂)₁₀—CH₃, —(CH₂)₁₁—CH₃, —(CH₂)₁₂—CH₃, —(CH₂)₁₃—CH₃, —(CH₂)₁₄—CH₃, —(CH₂)₁₅—CH₃, —(CH₂)₁₆—CH₃, —(CH₂)₁₇—CH₃, —(CH₂)₁₈—CH₃, —(CH₂)₁₉—CH₃, —(CH₂)₂₀—CH₃, —(CH₂)₂₁—CH₃, —(CH₂)₂₂—CH₃, —(CH₂)₂₃—CH₃, —(CH₂)₂₄—CH₃, —(CH₂)₂₅—CH₃, —(CH₂)₂₆—CH₃, —(CH₂)₂₇—CH₃, —(CH₂)₂₈—CH₃, —(CH₂)₂₉—CH₃, —(CH₂)₃₀—CH₃, —(CH₂)₃₁—CH₃, —(CH₂)₃₂—CH₃, —(CH₂)₃₃—CH₃, —(CH₂)₃₄—CH₃, —(CH₂)₃₅—CH₃, —(CH₂)₃₆—CH₃, —(CH₂)₃₇—CH₃, —(CH₂)₃₈—CH₃, or —(CH₂)₃₉—CH₃.

43. The pharmaceutical composition of any one of claims 34 to 36, wherein the albumin binding group is capable of covalently binding to the free thiol (Cys 34) or free —NH₂ (lysine(s)) of human serum albumin.

44. The pharmaceutical composition of claim 43, wherein the albumin binding group comprises a maleimide group, a halogenacetamide group, a halogenacetyl group, a halogenacetate group, a pyridinylthio group, a vinylcarbonyl group, an aziridinyl group, a disulfide group, an acetylene group, a hydroxysuccinimide group or a thiol group.

45. The pharmaceutical composition of claim 43 or claim 44, wherein the albumin binding group comprises a maleimide group:

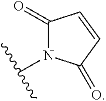 [SMILES: *N1C(=O)C=CC1=O]

46. The pharmaceutical composition of claim 45, wherein L is absent, 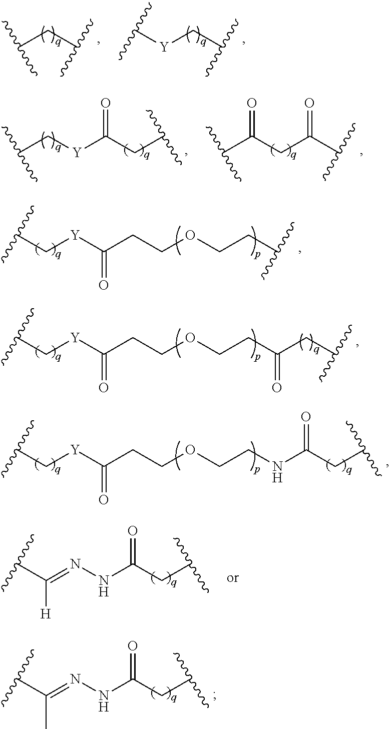
wherein Y is O or NH; each q is independently selected from 0, 1, 2, 3, 4, and 5; and p is 0, 1, 2, 3 or 4.

47. The pharmaceutical composition of claim 45, wherein -L-Ab is: 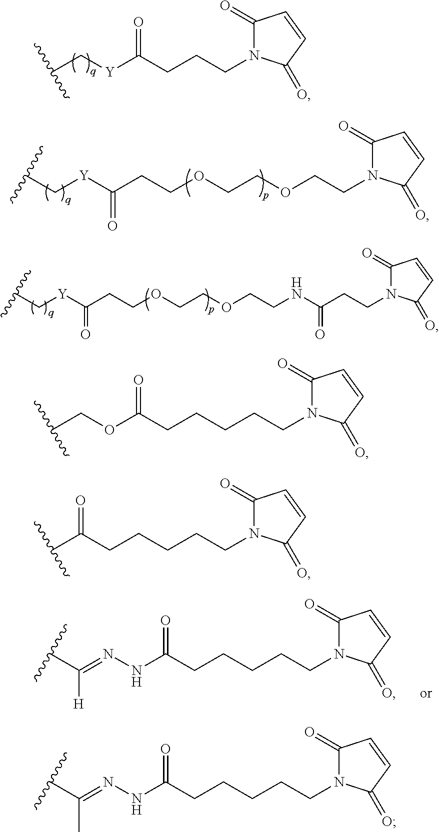
wherein Y is O or NH; q is 0, 1, 2 or 3; and p is 0, 1, 2 or 3.
